(19) United States
(12) Patent Application Publication
(10) Pub. No.: US 2025/0207163 A1
(43) Pub. Date: Jun. 26, 2025

(54) METHODS AND APPARATUS FOR SYNTHESIZING NUCLEIC ACIDS

(71) Applicant: Molecular Assemblies, Inc., San Diego, CA (US)

(72) Inventors: Patrycja A. Hopkins, San Diego, CA (US); Shahien Shahsavari, San Diego, CA (US); Vagarshak Begoyan, San Diego, CA (US); Olive J. Njuma, San Diego, CA (US); Holly S. Debenham, San Diego, CA (US); J. William Efcavitch, San Carlos, CA (US); Kim F. Albizati, San Diego, CA (US)

(21) Appl. No.: 18/850,185

(22) PCT Filed: Mar. 24, 2023
(86) PCT No.: PCT/US2023/016226

§ 371 Date: Sep. 24, 2024

Related U.S. Application Data
(60) Provisional application No. 63/323,766, filed on Mar. 25, 2022.

(51) Int. Cl. C12P 19/34; C07H 21/02

(52) U.S. Cl. CPC C12P 19/34; C07H 21/02

(57) ABSTRACT

Methods for the synthesis of polynucleotides using polymerases nucleotide analogs with labile terminator groups that allow stepwise addition of nucleotides are described. These nucleotide analogs may have a modification on 3′, 2′, or 3′-2′-bridged hydroxyl groups or on nucleobase moieties and can be used in the synthesis of natural or modified polynucleotides suitable for use in biological systems. The labile terminator groups are removed under mild conditions or by enzymes. They are accepted as substrates by polymerases. The synthesis may be carried out on a surface.

### FIELD OF THE INVENTION

The invention relates to methods and apparatus for synthesizing polynucleotides (de novo), and specifically ribonucleic acids, with a desired sequence and without the need for a template.

### BACKGROUND

Genomic manipulation requires tools for determining genomic content as well as for constructing desired genetic materials. The tools for determining the content of genomic material have made it possible to sequence an entire human genome in about one day for under $1,000. (See Life Technologies, Press Release: Benchtop Ion Proton Sequencer, Jan. 10, 2012). In contrast, the tools for constructing desired genetic materials, e.g., de novo DNA synthesis, have not improved at the same pace. As a point of reference, over the past 25 years, the cost (per base) of de novo small nucleic acid synthesis has dropped 10-fold, while the cost (per base) of nucleic acid sequencing has dropped over 10,000,000-fold. The lack of progress in DNA synthesis now limits the pace of translational genomics, i.e., whereby the role of individual sequence variations is determined and used to develop therapeutic treatments.

Currently, most de novo nucleic acid sequences, including RNA, are synthesized using solid phase phosphoramidite-techniques developed more than 30 years ago. The technique involves the sequential de-protection and synthesis of sequences built from phosphoramidite reagents corresponding to natural (or non-natural) nucleic acid bases. Phosphoramidite nucleic acid synthesis is length-limited, however, in that nucleic acids greater than 200 base pairs (bp) in length experience high rates of breakage and side reactions. With RNA synthesis, the length is even shorter and the synthesis process more challenging.

Additionally, phosphoramidite synthesis produces toxic by-products and the disposal of this waste limits the availability of nucleic acid synthesizers and increases the costs of contract oligo production. (It is estimated that the annual demand for oligonucleotide synthesis is responsible for greater than 300,000 gallons of hazardous chemical waste, including acetonitrile, trichloroacetic acid, toluene, tetrahydrofuran, and pyridine. See LeProust et al., Nucleic Acids Res., vol. 38(8), p.2522-2540, (2010), incorporated by reference herein in its entirety). The lack of progress in RNA synthesis now limits the pace of translational genomics, i.e., whereby the role of individual sequence variations is determined and used to develop therapeutic treatments. Thus, there is a need for more efficient and cost-effective methods for oligonucleotide synthesis, especially for the more challenging synthesis of ribonucleic acids.

### SUMMARY

The invention provides improved methods for nucleic acid synthesis, and especially ribonucleic acid synthesis. Methods of the invention provide faster and longer de novo synthesis of polynucleotides. As such, the invention dramatically reduces the overall cost of synthesizing custom nucleic acids. Methods of the invention are directed to template-independent synthesis of polynucleotides using a polymerase to incorporate nucleotide analogs coupled to an inhibitor via a cleavable linker. The inhibitor can be modified at 2′-, at 3′-, or at both 2′ and 3′-hydroxyl groups that are cleavable under mild enzymatic or non-enzymatic conditions. The use of mild cleaving conditions, or in some cases modified 2′ groups can help prevent incidental strand scission that can occur during the removal of 3′-linked terminators in the presence of an unprotected 2′hydroxyl. Because of the inhibitor, synthesis pauses with the addition of each new base, whereupon the linker is cleaved, separating the inhibitor and leaving a polynucleotide that is essentially identical to a naturally occurring nucleotide (i.e., is recognized by the enzyme as a substrate for further nucleotide incorporation).

In particular, the invention provides a renewable substrate for template-independent nucleic acid synthesis. The invention is applicable to any nucleic acid (e.g., DNA or RNA, including derivatives thereof) but will be exemplified for use with RNA herein. De novo synthesis begins with a nucleic acid initiator that is bound to a solid support. In the presence of a suitable enzyme, e.g., a polymerase, e.g., a terminal deoxynucleotidyl transferase (TdT), or polymerase theta, nucleotide analogs are added to the nucleic acid initiator in order to create an oligonucleotide. It is preferable that the nucleotide analogs include removable terminating groups that cause the enzymatic addition to stop after the addition of one nucleotide. A removable terminating group can be linked to the base portion of the nucleic acid and/or to the 2′ and/or 3′ hydroxyl of the nucleic acid. Deblocking of the terminating group and/or the 2′ and/or 3′ blocking group, creates a new substrate for the enzyme. With subsequent addition of a new nucleotide or nucleotide analog, the oligonucleotide is extended.

The terminating groups may include, for example, charged moieties or steric inhibitors. In general, a terminating group may be a modification that prevents polymerases from achieving a functional conformation that allows for another nucleotide addition. Such modifications may include, for example, ethers, pseudohalogens, nitrates, nitrites, sulfonates, sulfinates, phosphates, phosphites, phosphides, and/or esters. The modifications are linked to nucleotide analogs at various position as described below. These terminating groups can be removed under mild condition or by enzymes. If harsher conditions are required, a modification or protecting group may be present on the 2′ hydroxy to prevent unintended cleavage or scission of the nascent nucleic acid strand. The 2′ modification or protecting group can be linked such that the 2′ modification or protecting group remains in place upon removal of the 3′ blocking group or base-linked terminating group. In such instances, unlike the 3′ blocking group or base-linked terminating group, the 2′ modification or protecting group will not interfere with strand synthesis or incorporation of additional nucleotides or nucleotide analogs. At the end of nucleic acid synthesis (e.g., ribooligonucleotide synthesis), all of the 2′ modifications or protecting groups remaining on the synthesized strand can be globally removed or deblocked to leave a natural-appearing nucleic acid strand. In some instances, the 2′ modifications are not removable and are meant to remain in the final product strand. In some instances, the desired ribonucleic acid may consist of sequence specific combinations of either 2′-hydroxyl and several different or the same 2′ modified ribonucleotides.

In some instances, the nucleic acid initiator comprises a 3′ moiety that is a substrate for the enzyme. A releasing agent is used to deprotect the 3′ moiety, thereby releasing the oligonucleotide. The 3′ moiety, the nucleic acid initiator, and the solid support are reusable after the release of the nascent oligonucleotide.

The invention additionally includes an apparatus that utilizes methods of the invention for the production of custom polynucleotides. An apparatus of the invention includes one or more bioreactors providing aqueous conditions and a plurality of sources of nucleotide analogs. The bioreactor may be e.g., a reservoir, a flow cell, or a multi-well plate. The bioreactor may include a solid support having a nucleic acid initiator and a cleavable 3′ moiety. Starting from the solid support, the polynucleotides are grown in the reactor by adding successive nucleotides via the natural activity of a polymerase, e.g., a terminal deoxynucleotidyl transferase (TdT)

or any other enzyme that elongates DNA or RNA strands without template direction. Upon cleavage of the linker, a natural polynucleotide is released from the solid support. Once the sequence is complete, the support is cleaved away, or the 3′ moiety is contacted with a releasing agent, leaving a polynucleotide essentially equivalent to that found in nature. In some embodiments, the apparatus is designed to recycle nucleotide analog solutions by recovering the solutions after nucleotide addition and reusing solutions for subsequence nucleotide addition. Thus, less waste is produced, and the overall cost per base is reduced as compared to state-of-the-art methods. In certain embodiments, a bioreactor may include a microfluidic device and/or use inkjet printing technology.

In embodiments using a 3′-O-blocked nucleotide analog, the 3′-O-blocking groups are typically small and easily removed, thus allowing use with engineered enzymes having modified active sites. For example, the 3′-O-blocking groups may comprise azidomethyl, amino, phosphates or allyl groups.

In some embodiments, oligonucleotide synthesis may include introduction of a 3′ exonuclease to the one or more synthesized oligonucleotides after each nucleotide analog addition, but before cleaving the terminating group. The terminating group blocks the 3′ exonuclease from acting on any oligonucleotide to which a nucleotide analog has been added, while oligonucleotides that have not successfully added an analog containing a terminator are removed by the 3′ exonuclease. In this manner, the invention allows for in-process quality control and may eliminate the need for post-synthesis purification.

Aspects of the invention can include methods for synthesizing an oligonucleotide including exposing a nucleic acid attached to a solid support to a nucleotide analog in the presence of a polymerase and in the absence of a nucleic acid template to incorporate the nucleotide analog into the nucleic acid, wherein, after incorporation, the nucleotide analog comprises a modification that prevents the polymerase from catalyzing addition of a subsequent natural nucleotide or nucleotide analog, and wherein the modification is selected from the group consisting of a 3′-O-blocking group and 2′-hydroxyl, a 3′-O-blocking group and 2′-modifying group, a 3′-hydroxyl and 2′-O-blocking group, and a 2′,3′-bridged hydroxyl group; and altering the modification to the incorporated nucleotide analog to permit incorporation of a subsequent natural nucleotide or nucleotide analog under altering conditions that prevent strand scission.

The altering conditions can include exposure to an enzyme to remove the 3′-O-blocking group. In certain embodiments, the altering conditions may include exposure to a non-enzymatic reagent to remove the 3′-O-blocking group. The nucleotidyl transfer enzyme can be polymerase theta or a variant thereof.

In certain embodiments, the 3′-O-blocking group can include one or more ethers, pseudohalogens, nitrates, nitrites, sulfinates, sulfonates, phosphates, phosphites, phosphides, or esters. The 2′-O-blocking group may include one or more ethers, pseudohalogens, nitrates, nitrites, sulfinates, sulfonates, phosphates, phosphites, phosphides, or esters. The 2′-modifying blocking group may include O-alkyl, methoxy, amino, fluoro, chloro, or bromo.

In various embodiments, the nucleotide analog may include a base modification selected from the group consisting of N1-methyladenosine, N6-methyladenosine, N6-methyl-2-aminoadenosine, 5-methyluridine, N1-methylpseudouridine, pseudouridine, 5-hydroxymethyluridine, 5-methylcytidine, 5-hydroxymethylcytidine, N7-methylguanosine, inosine, 2-thiouridine, 6-thioguanosine, 4-thiourdine, xanthosine, and N,N-dimethyladenosine. In some instances, the desired ribonucleic acid may consist of sequence specific combinations of base modifications bearing 2′hydroxyl and several different or the same 2′ modified ribonucleotides.

In certain aspects, methods of the invention may include synthesizing a ribooligonucleotide by exposing a nucleic acid attached to a solid support to a nucleotide analog in the presence of a polymerase enzyme and in the absence of a nucleic acid template to incorporate the nucleotide analog into the nucleic acid, wherein, after incorporation, the nucleotide analog comprises a modification that prevents the polymerase from catalyzing addition of a subsequent natural nucleotide or nucleotide analog, and wherein the modification comprises a 3′-O-blocking group or a 2′-O-blocking group, a 3′-O-blocking group and a 2′-modifying group, or a 3′-2′ bridged hydroxyl group; altering the modification to the incorporated nucleotide analog to permit incorporation of a subsequent natural nucleotide or nucleotide analog such that a 2′-O-blocking group remains on the incorporated nucleotide analog; repeating the exposing and altering steps until a desired ribooligonucleotide is synthesized; and treating the synthesized oligoribonucleotide to remove all remaining 2′-O-blocking groups from incorporated nucleotide analogs.

Other aspects of the invention are apparent to the skilled artisan upon consideration of the following figures and detailed description.

### BRIEF DESCRIPTION OF THE DRAWINGS

FIG. 1 shows an exemplary RNA synthesis cycle utilizing any triphosphate analog having a cleavable terminator linker at 3′ position and 2′ is hydroxyl group.

FIG. 2 shows an exemplary RNA synthesis cycle utilizing any triphosphate analog having a cleavable terminator linker at 3′ position and 2′ is any other substitution such as amino, methyl, methoxy, halide, etc.

FIG. 3 shows an exemplary RNA synthesis cycle utilizing any triphosphate analog having a cleavable terminator linker at 2′ and 3′ position, where 3′ modification is cleaved at each step and 2′ modification is cleaved globally at the end of RNA synthesis.

FIG. 4 shows an exemplary RNA synthesis cycle utilizing any triphosphate analog having a cleavable terminator linker at 2′,3′ bridged position.

### DETAILED DESCRIPTION

The invention provides improved methods for synthesizing polynucleotides, such as DNA and RNA, using enzymes and nucleic acid analogs. Using the disclosed methods, specific sequences of polynucleotides can be synthesized de novo, base by base, in an aqueous environment, and without the use of a nucleic acid template. Systems and methods described herein are particularly useful in synthesizing ribooligonucleotides that can be prone to strand scission during synthesis.

Nucleotide analogs may have a 3′-O and/or a 2′-0 modification or blocking, protecting, or terminating group. In any case, the blocking, protecting or terminating group is designed to not leave behind substantial residue, i.e., designed to leave behind “scarless” nucleotides that are recognized as “natural” nucleotides by the enzyme. Thus, at the conclusion of the synthesis, upon removal of the last blocking group, the synthesized polynucleotide is chemically and structurally equivalent to the native polynucleotide with the same sequence. In other instances, at the conclusion of the synthesis, upon removal of the last blocking group, the synthesized polynucleotide contains sequence specific 2′ modified analogs affording specific physical or biochemical properties to the desired polynucleotide. The synthetic polynucleotide can, thus, be incorporated into living systems without concern that the synthesized polynucleotide will interfere with biochemical pathways or metabolism. In various embodiments, 3′-0 and/or a 2′-0 modification or blocking, protecting, or terminating groups may be used with base-linked inhibitory, blocking, terminating, or protecting groups (e.g., including self-eliminating linkers) and enzymes (e.g., modified TdT enzymes or polymerase theta) as described in U.S. patent application Ser. Nos. 16/841,214; 16/891,449; and 15/926,642 the content of each of which is incorporated herein by reference.

The process and analogs of the invention are used for the non-template mediated enzymatic synthesis of oligo- and oligoribonucleotides especially of long sequences (<5000 nt). Products can be single stranded or partially double stranded depending upon the initiator used. The synthesis of long oligonucleotides requires high efficiency incorporation and high efficiency of reversible terminator removal. The initiator bound to the solid support consists of a short, single strand RNA or DNA sequence that is either a short piece of the user defined sequence or a universal initiator from which the user defined single strand product is removed.

In certain aspects nucleotide analogs may be of the form:

### NTP-Inhibitor

where NTP is a nucleotide triphosphate (i.e. rNTP), inhibitor is a group that prevents the enzyme from incorporating subsequent nucleotides. At each step, a new nucleotide analog is incorporated into the growing polynucleotide chain, whereupon the enzyme is blocked from adding an additional nucleotide by the inhibitor group. Once the enzyme has stopped, the excess nucleotide analog can be removed from the growing chain, the inhibitor can be cleaved from the NTP under mild conditions, and new nucleotide analogs can be introduced in order to add the next nucleotide to the chain. By repeating the steps sequentially, it is possible to quickly construct a nucleotide sequence of a desired length and sequence. In addition, methods may make use of an initiator sequence that is a substrate for polymerase. The initiator may be attached to solid support and serve as a binding site for the enzyme. The initiator is preferably a universal initiator for the enzyme, such a homopolymer sequence and is recyclable on the solid support, with the synthesized oligonucleotide being cleavable from the initiator leaving the initiator a suitable substrate for synthesis of another oligonucleotide.

Methods of the invention are well-suited to a variety of applications that currently use synthetic ribonucleic acids, e.g., phosphoramidite-synthesized RNA oligos. For example, polynucleotides synthesized with systems and methods of invention can be used as primers for nucleic acid amplification, hybridization probes for detection of specific markers, and for incorporation into plasmids for genetic engineering. However, because the disclosed methods produce longer synthetic strings of nucleotides, at a faster rate, the disclosed methods also lend themselves to high-throughput applications, such as screening for expression of genetic variations in cellular assays, as well as synthetic biology. Furthermore, systems and methods of the invention can provide the functionality needed for next-generation applications, such as using RNA as synthetic write/read memory, or creating macroscopic materials synthesized completely (or partially) from RNA. Various schemes for nucleic acid memory reading and writing compatible with systems and methods of the invention are described in U.S. application Ser. No. 16/393,510 the content of which is incorporated herein by reference.

The methods and systems described herein provide for synthesis of polynucleotides, including ribonucleic acids (RNA). While synthetic pathways for “natural” nucleotides, such as RNA, are described in the context of the common nucleic acid bases, e.g., adenine (A), guanine (G), cytosine (C) and uracil (U), it is to be understood that the methods of invention can be and will be applied to so-called “non-natural” nucleotides, including but not limiting to nucleotides incorporation universal bases such as 3-nitropyrrole nucleosides, 5-nitroindole nucleosides, alpha phosphorothioate, phosphorothioate nucleotide triphosphates, or purine and pyrimidine conjugates that have other desirable properties, such as fluorescence. Other examples of purine and pyrimidine bases include pyrazolo[3,4-d]pyrimidines, 5-methylcytosine (5-me-C), 5-hydroxylmethylcytosine, xanthine, hypoxanthine, 2-aminoadenine, 6-methyl or another alkyl derivatives od adenine and guanine, 2-propyl and other alkyl derivatives of adenine and guanine, 2-thiouracil, 2-thiocytosine, 5-propyl uracil and cytosine, 6-azo uracil and cytosine, 5-uracil (pseudouracil), 4-thiouracil, 8-halo (e.g., 8-bromo), 8-amino, 8-thiol, 8-thioalkyl, 8-hydroxyl and other 8-substituted adenines and guanines, 5-halo particularly 5-bromo, 5-trifluoromethyl and other 5-substitued uracils and cytosines, 7-methylguanine and 7-methyadenine, 8-azaguanine and 8-azaadenine, dezaguanine, 7-deazaguanine, 3-deazaguanine, deazaadenine, 7-deazaadenine, 3-deazaadenine, pyrazolo[3,4-d]pyridimes, imidazole[1,5-a]1,3,5 triazanones, 9-deazapurines, imidazo[4,5-d]pyrazines, thiazolo[4,5-d]pyrimidines, pyrazin-2-ones, 1,2,4-triazine, pyridazine, and 1,3,5-triazine. In some instances, it may be useful to produce nucleotide sequences having unreactive, but approximately equivalent bases, i.e., bases that do not react with other proteins, i.e. transcriptases, thus allowing the influence of sequence information to be decoupled from the structural effects of the bases.

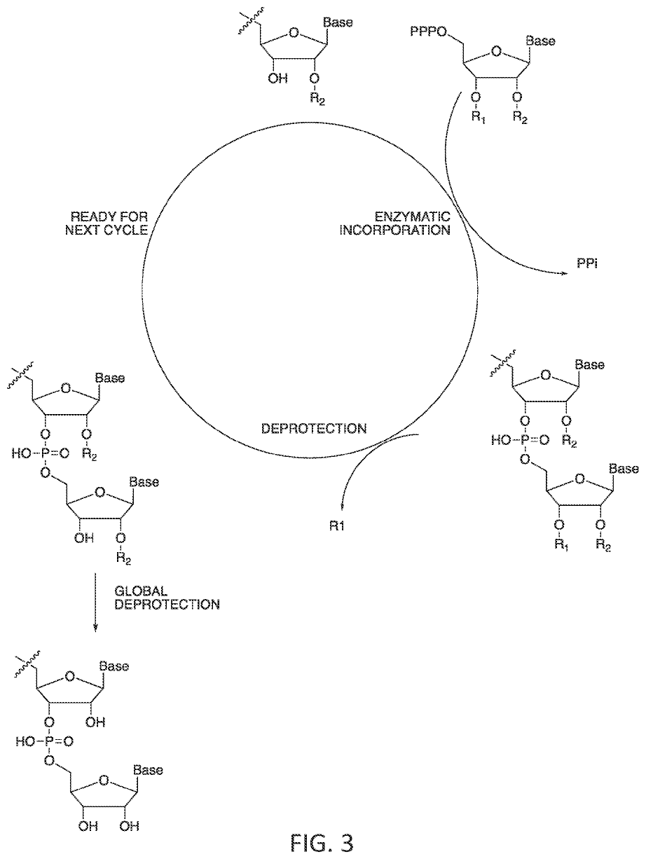

As discussed above, inhibiting, blocking, or protected groups or modifications can be positioned at various combinations of the base and the 2′ and/or 3′ carbonor hydroxyl group of the sugar of the nucleotide analog. In certain embodiments, a cleavable blocking modification is made to the 3′ oxygen that prevents the nucleotide analog from acting as a substrate for additional nucleotide (natural or analog) incorporation by polymerase. Accordingly, synthesis of a nucleic acid strand will pause after each incorporation until the blocking modification is removed. With certain cleavage conditions, especially with sensitive ribooligonucleotide synthesis, there is a risk of the nascent strand being cleaved when the 2′ hydroxyl is unprotected. Accordingly, in certain embodiments (see, e.g., FIG. 3), a separate protecting, blocking, or inhibiting group or modification is included on the 2′ carbon or oxygen of the nucleotide analog. That 2′ modification does not interfere with oligo synthesis by the polymerase (unlike the 3′ modification) and is cleavable under different conditions than the 3′ modification. Accordingly, as the strand is synthesized, the 2′ modifications remain on the incorporated nucleotide analogs, thereby protecting the strand against unwanted scission. After the strand is complete, the 2′ modifications can be removed in bulk from the entire synthesized strand, leaving a natural appearing and functioning nucleic acid.

Removing or altering the 3′ or 2′ sugar modifications can include both enzymatic and non-enzymatic treatments. Altering the modification comprises exposing the incorporated nucleotide analog to an enzyme to remove the 3′-O-blocking group.

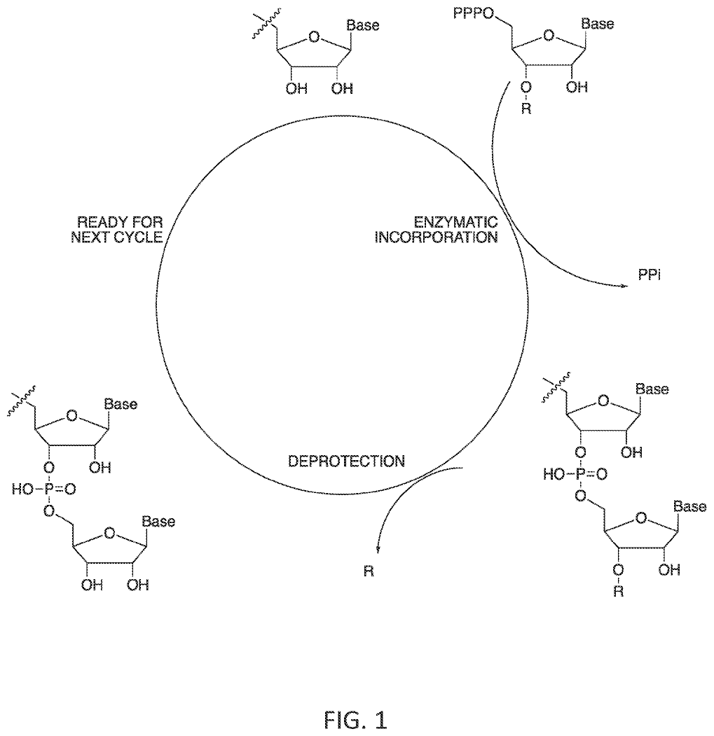

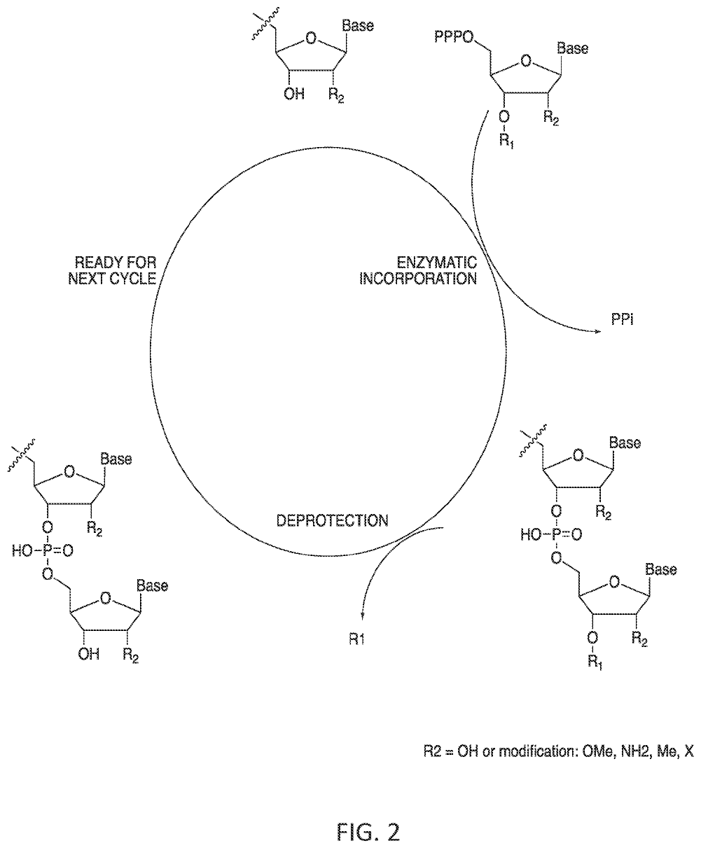

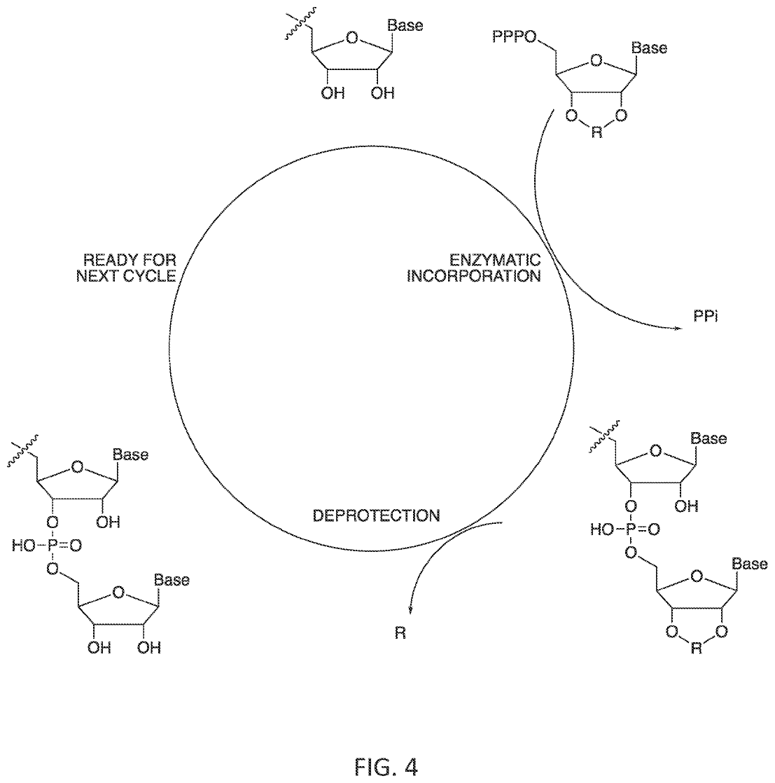

An exemplary RNA synthesis cycle using a triphosphate analog having a cleavable 3′-O-blocking group is shown in FIG. 1. The 2′ hydroxyl is unmodified and, accordingly, the cleavage mechanism for the 3′-O-blocking group should be mild to prevent unwanted strand scission during deprotecting steps. FIG. 2 shows an exemplary RNA synthesis cycle with a triphosphate analog having a cleavable 3′-O-blocking group and a 2′ modification (e.g., any other substitution such as amino, methyl, methoxy, halide, etc.). FIG. 3 shows an exemplary RNA synthesis cycle using a triphosphate analog having cleavable 2′-O and 3′-O-blocking groups where the 3′ modification is cleaved at each step and 2′ modification is cleaved globally at the end of RNA synthesis. FIG. 4 shows an exemplary RNA synthesis cycle using a triphosphate analog having a cleavable blocking group at a 2′ 3′ bridged position.

3′-O-blocking groups may include ethers, pseudohalogens, nitrates, nitrites, sulfinates, sulfonates, phosphates, phosphites, phosphides, and esters. 2′-O-blocking groups may include ethers, pseudohalogens, nitrates, nitrites, sulfinates, sulfonates, phosphates, phosphites, phosphides, and esters. In certain embodiments, 2′ modifying groups may include O-alkyl, methoxy, amino, fluoro, chloro, or bromo. Exemplary base modifications for incorporation can include 2-thio-2′-O-methyluridine, 3,2′-O-dimethyluridine 2′-O-methyluridine, 5-oxyacetic acid methyl ester, 5-carboxymethylaminomethyl-2′-O-methyluridine, 5-methoxycarbonylmethyl-2′-O-methyluridine, 5-(carboxyhydroxymethyl)-2′-O-methyluridine methyl ester, 5-carbamoylmethyl-2′-O-methyluridine 5-(isopentenylaminomethyl)-2′-O-methyluridine, 5,2′-O-dimethyluridine 2′-O-methylpseudouridine, 2′-O-methyluridine, 1-methyl-3-(3-amino-3-carboxypropyl)pseudouridine, 1-methylpseudouridine, 5-aminomethyl-2-selenouridine, 5-methylaminomethyl-2-selenouridine, 5-carboxymethylaminomethyl-2-selenouridine, 2-selenouridine, 5-aminomethyl-2-geranylthiouridine, 5-methylaminomethyl-2-geranylthiouridine, 5-carboxymethylaminomethyl-2-geranylthiouridine, 2-geranylthiouridine 5-aminomethyl-2-thiouridine, 5-methylaminomethyl-2-thiouridine, 5-carboxymethylaminomethyl-2-thiouridine, 5-methoxycarbonylmethyl-2-thiouridine, 5-carbamoylmethyl-2-thiouridine, 5-carboxymethyl-2-thiouridine, 5-taurinomethyl-2-thiouridine, 5-(isopentenylaminomethyl)-2-thiouridine, 5-methyl-2-thiouridine, 2-thiouridine, 3-(3-amino-3-carboxypropyl)-5,6-dihydrouridine, 3-(3-amino-3-carboxypropyl)pseudouridine, 3-(3-amino-3-carboxypropyl)uridine, 3-methylpseudouridine, 3-methyluridine, 5-methoxyuridine, uridine 5-oxyacetic acid, uridine 5-oxyacetic acid methyl ester, 5-hydroxyuridine, 5-aminomethyluridine, 5-methylaminomethyluridine, 5-carboxymethylaminomethyluridine, 5-carboxyhydroxymethyluridine, 5-methoxycarbonylmethyluridine, 5-(carboxyhydroxymethyl)uridine, methyl ester 5-carboxymethyluridine, 5-carbamoylhydroxymethyluridine, 5-carbamoylmethyluridine, 5-taurinomethyluridine, 5-cyanomethyluridine, 5-(isopentenylaminomethyl)uridine 5-methyldihydrouridine, 5-methyluridine 4-thiouridine dihydrouridine pseudouridine, N4-acetyl-2′-O-methylcytidine, N4,N4,2′-O-trimethylcytidine, N4,2′-O-dimethylcytidine, 2′-O-methyl-5-hydroxymethylcytidine, 5,2′-O-dimethylcytidine, 5-formyl-2′-O-methylcytidine, 2′-O-methylcytidine, agmatidine, 2-lysidine, 2-thiocytidine, 3-methylcytidine, N4-acetylcytidine, N4,N4-dimethylcytidine, N4-methylcytidine, 5-hydroxycytidine, 5-hydroxymethylcytidine, 5-methylcytidine, 5-formylcytidine, 1,2′-O-dimethylinosine, 1,2′-O-dimethyladenosine, N6,N6,2′-O-trimethyladenosine, N6,2′-O-dimethyladenosine, 2′-O-methylinosine, 2′-O-methyladenosine, 1-methylinosine, 1-methyladenosine, 2-methylthiomethylenethio-N6-isopentenyl-adenosine, 2-methylthio-N6-(cis-hydroxyisopentenyl) adenosine, 2-methylthio-N6-isopentenyladenosine, 2-methylthio-N6-threonylcarbamoyladenosine, 2-methylthio-N6-hydroxynorvalylcarbamoyladenosine, 2-methylthio cyclic N6-threonylcarbamoyladenosine, hydroxy-N6-threonylcarbamoyladenosine, 2,8-dimethyladenosine, 2-methyladenosine, N6-(cis-hydroxyisopentenyl)adenosine, N6-isopentenyladenosine, 2-methylthio-N6-methyladenosine, N6-threonylcarbamoyladenosine, N6-hydroxynorvalylcarbamoyladenosine, N6-acetyladenosine, N6-glycinylcarbamoyladenosine, N6-methyl-N6-threonylcarbamoyladenosine, N6,N6-dimethyladenosine, N6-formyladenosine, N6-hydroxymethyladenosine, cyclic N6-threonylcarbamoyladenosine, N6-methyladenosine, 8-methyladenosine, inosine, 1,2′-O-dimethylguanosine, N2,N2,2′-O-trimethylguanosine, N2,7,2′-O-trimethylguanosine, N2,2′-O-dimethylguanosine, 2′-O-methylguanosine, 7-cyano-7-deazaguanosine, 7-aminomethyl-7-deazaguanosine, epoxyqueuosine, archaeosine, galactosyl-queuosine, glutamyl-queuosine, mannosyl-queuosine, queuosine, 1-methylguanosine, N2,N2,7-trimethylguanosine, N2,N2-dimethylguanosine, N2,7-dimethylguanosine, N2-methylguanosine, methylwyosine, 7-aminocarboxypropylwyosine, hydroxywybutosine, peroxywybutosine, wybutosine, 7-aminocarboxypropylwyosine methyl ester, wyosine, isowyosine, 7-aminocarboxypropyl-demethylwyosine, 4-demethylwyosine, 7-methylguanosine. [MODOMICS: a database of RNA modification pathways. 2021 update; Nucleic Acids Research, Volume 50, Issue D1, 7 Jan. 2022, Pages D231-D235] and including, but not limited to, the structures in Table 1 below.

TABLE 1
Exemplary Uridine modifications include, but are not limited to:

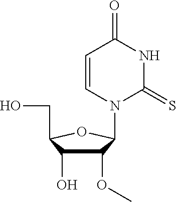

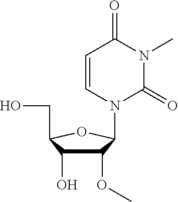

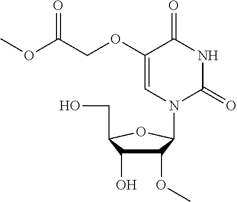

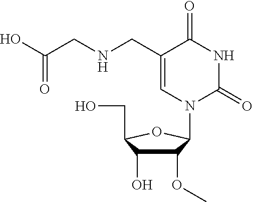

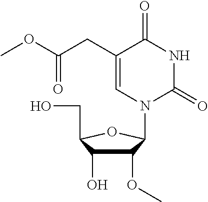

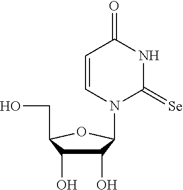

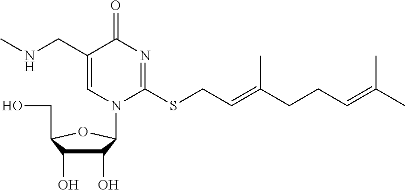

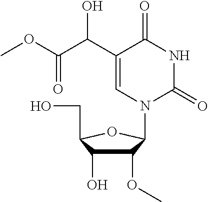

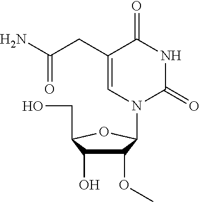

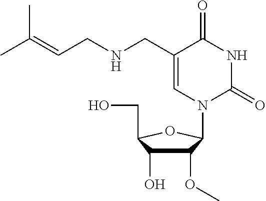

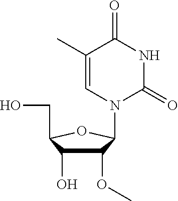

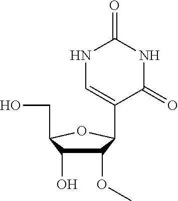

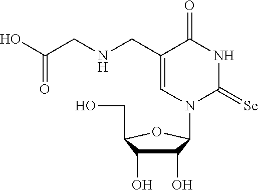

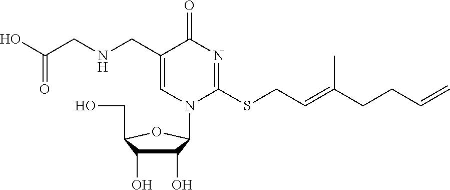

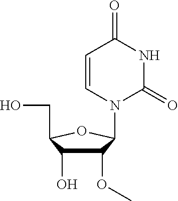

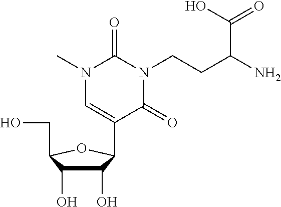

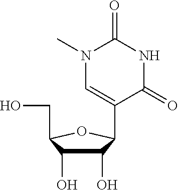

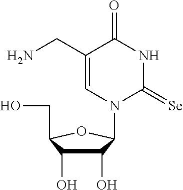

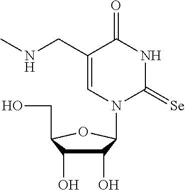

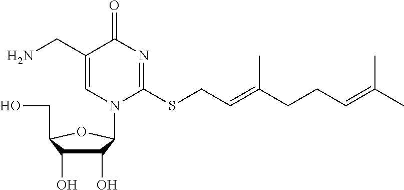

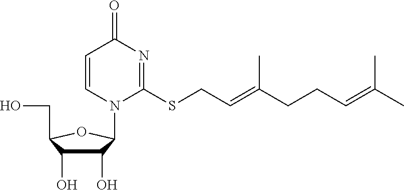

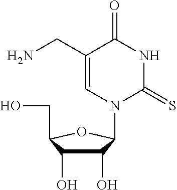

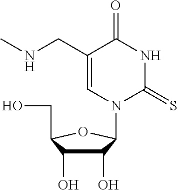

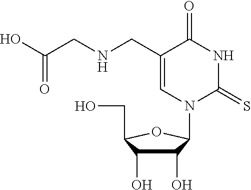

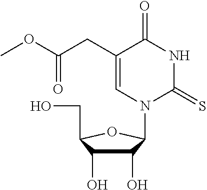

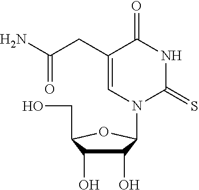

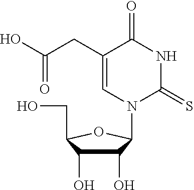

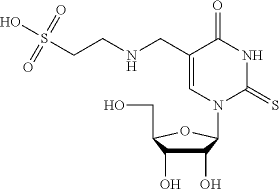

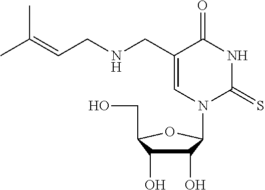

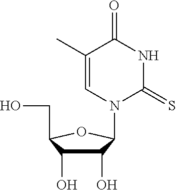

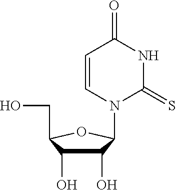

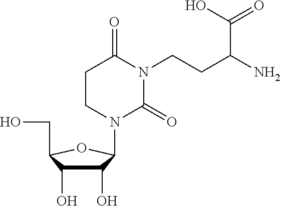

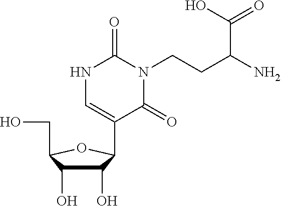

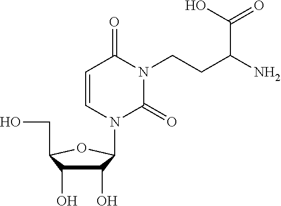

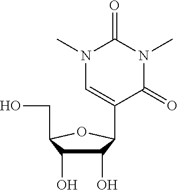

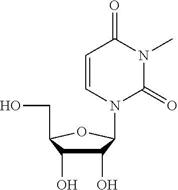

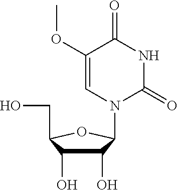

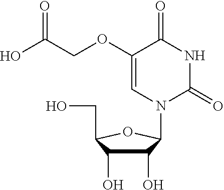

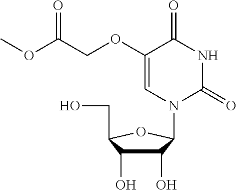

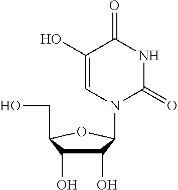

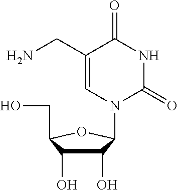

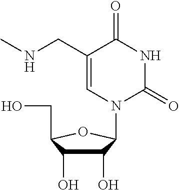

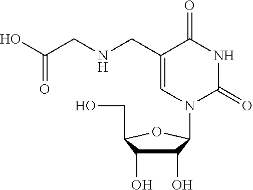

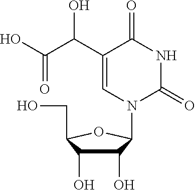

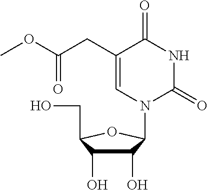

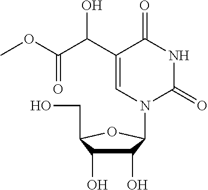

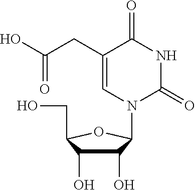

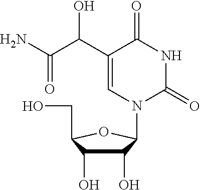

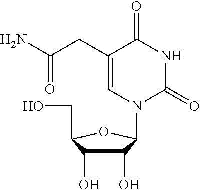

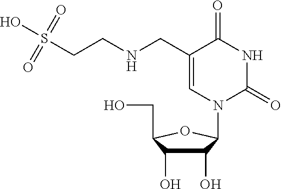

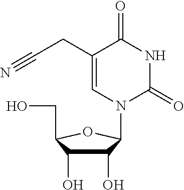

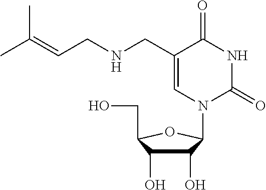

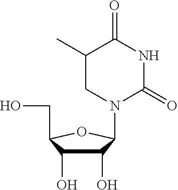

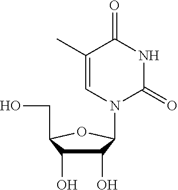

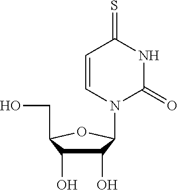

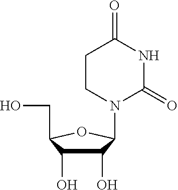

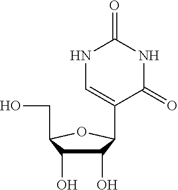

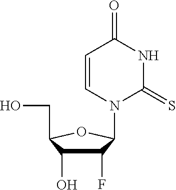

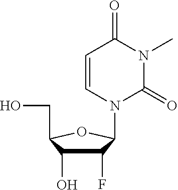

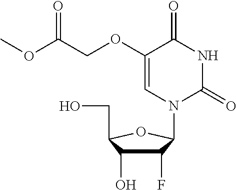

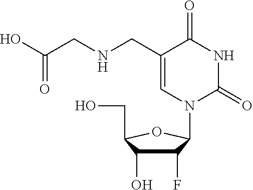

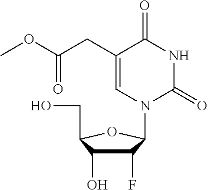

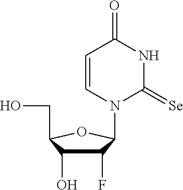

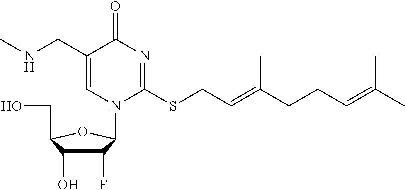

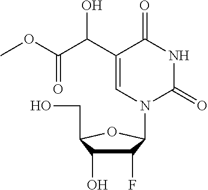

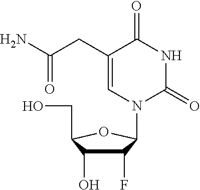

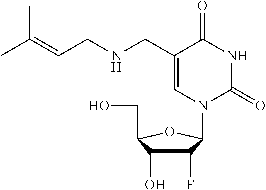

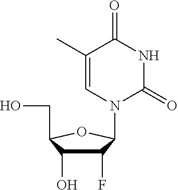

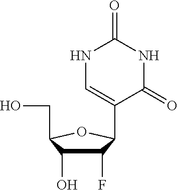

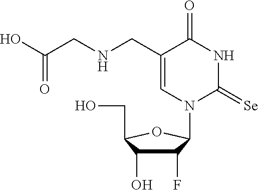

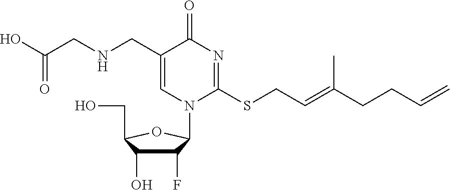

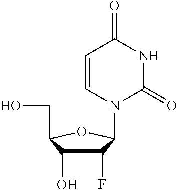

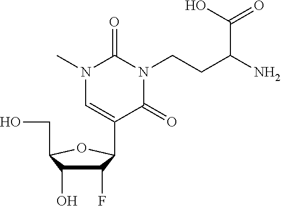

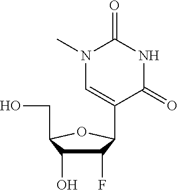

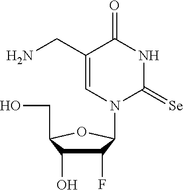

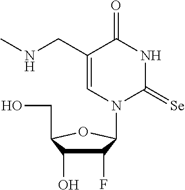

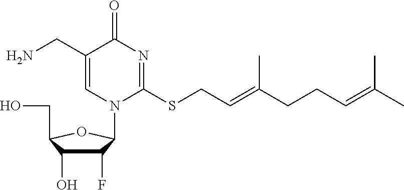

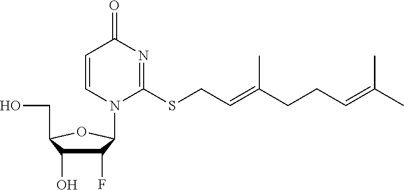

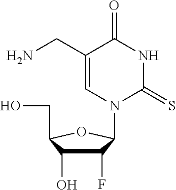

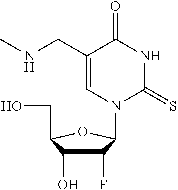

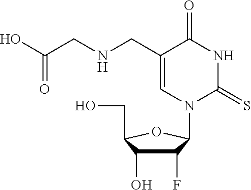

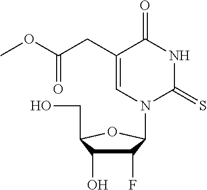

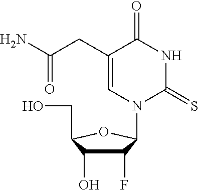

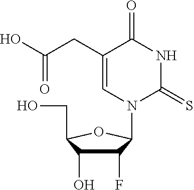

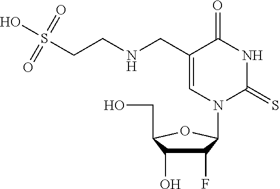

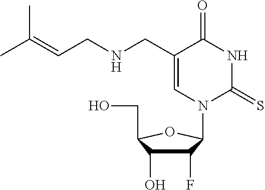

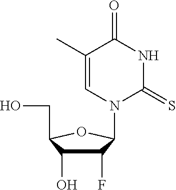

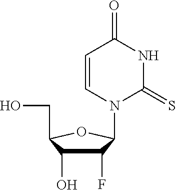

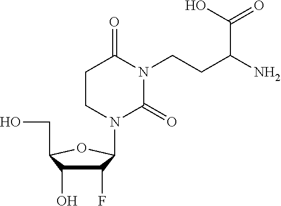

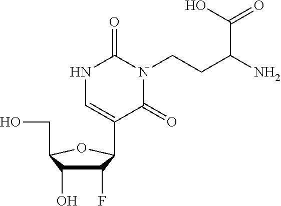

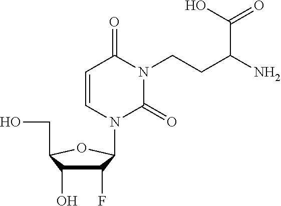

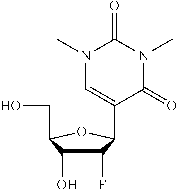

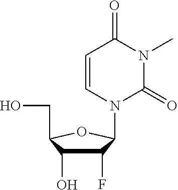

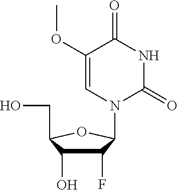

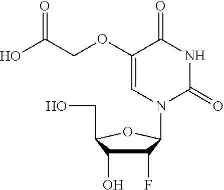

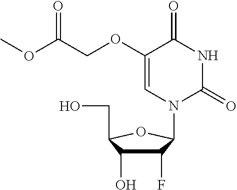

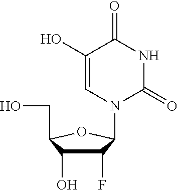

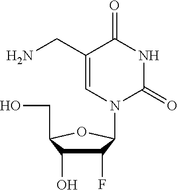

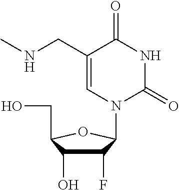

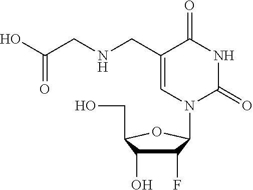

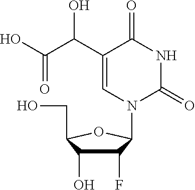

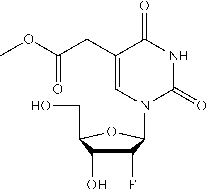

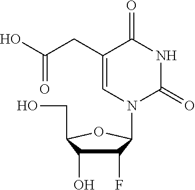

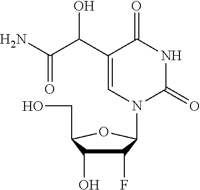

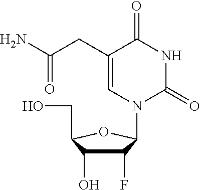

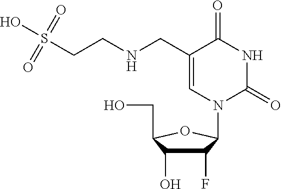

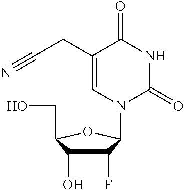

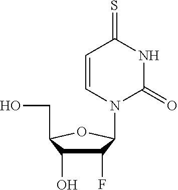

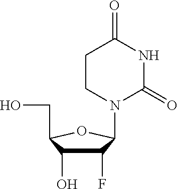

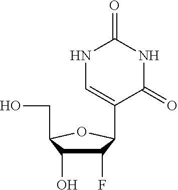

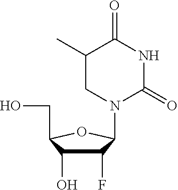

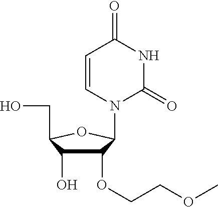

Exemplary adenosine modifications include, but are not limited to:

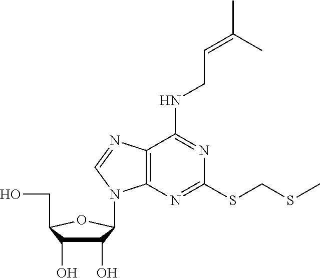

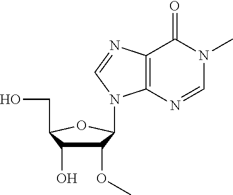

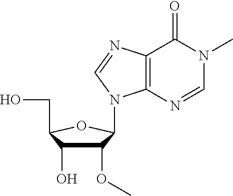

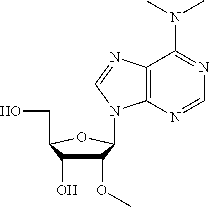

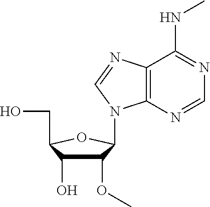

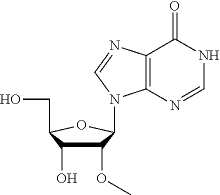

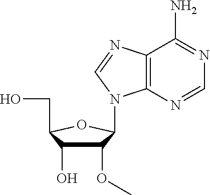

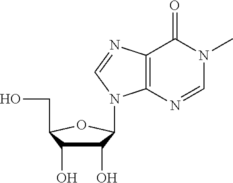

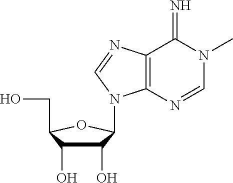

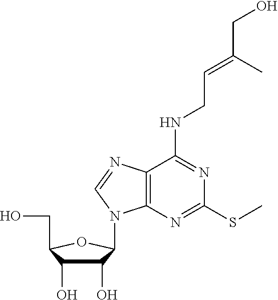

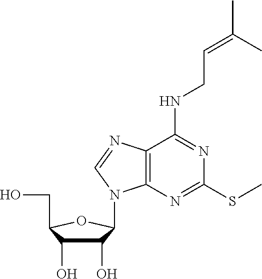

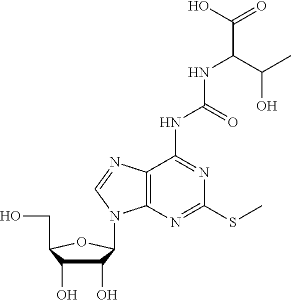

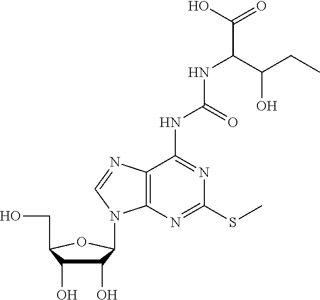

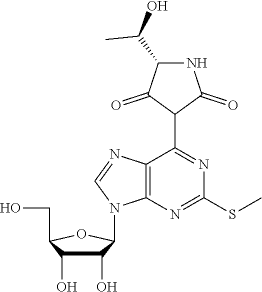

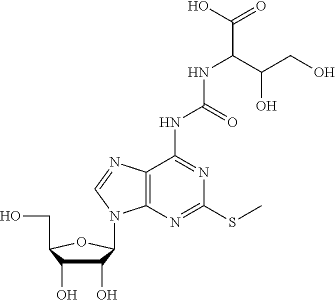

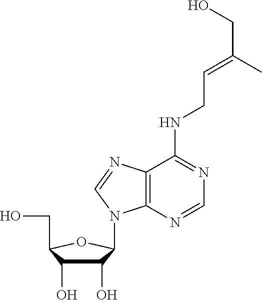

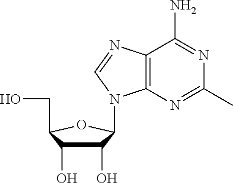

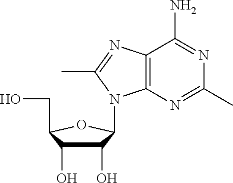

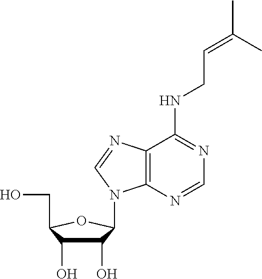

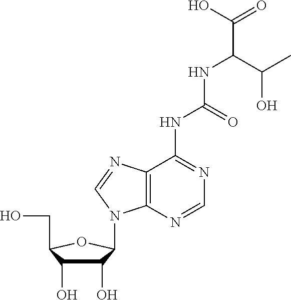

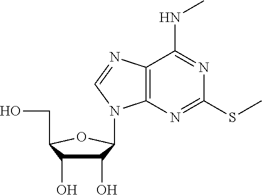

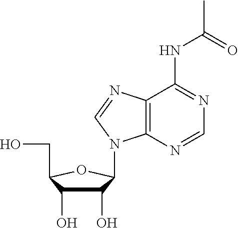

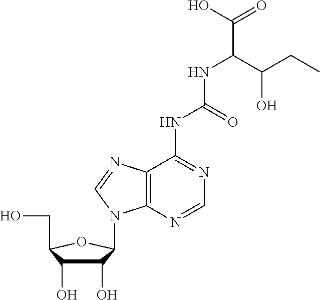

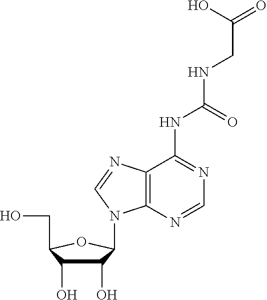

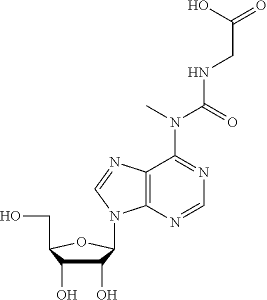

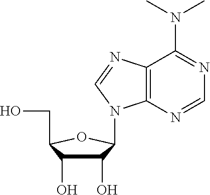

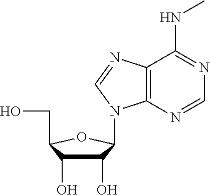

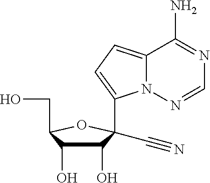

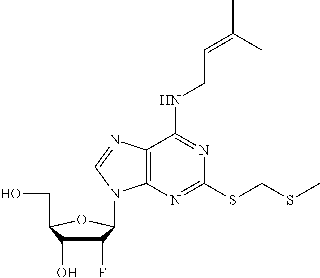

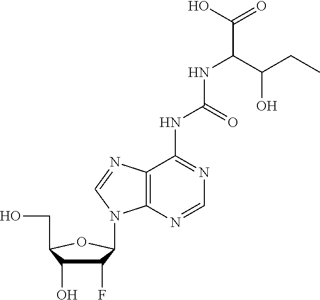

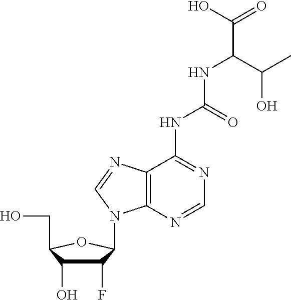

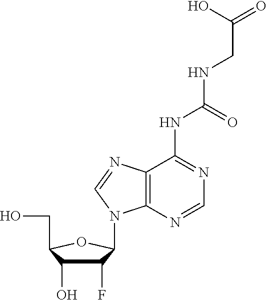

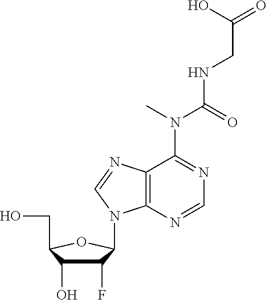

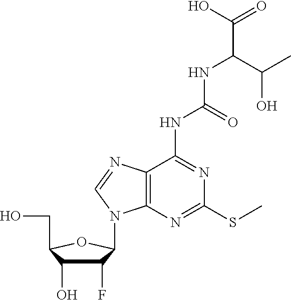

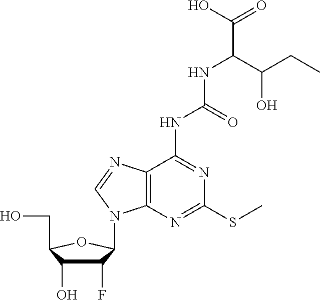

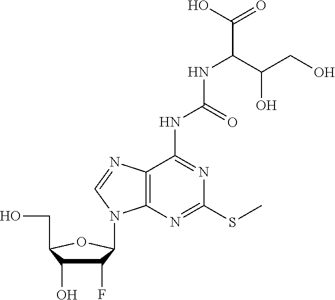

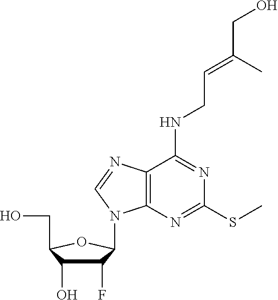

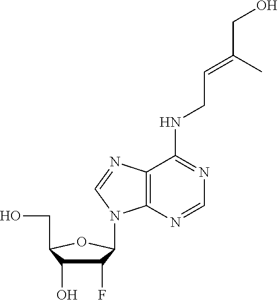

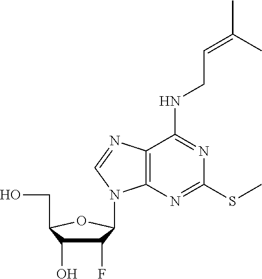

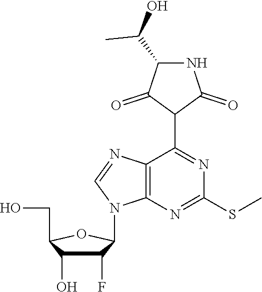

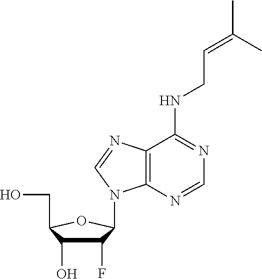

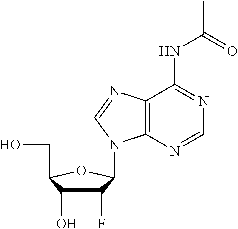

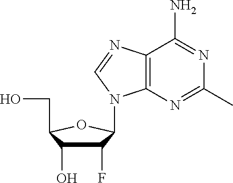

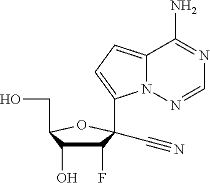

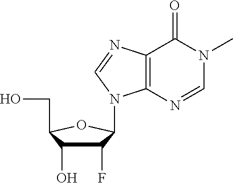

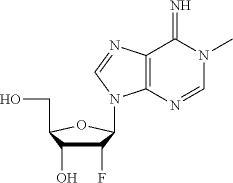

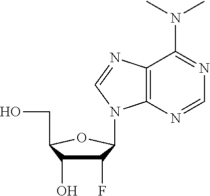

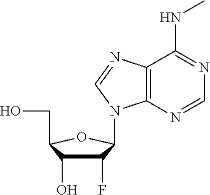

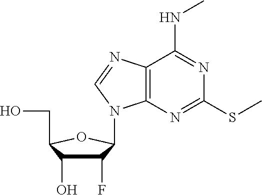

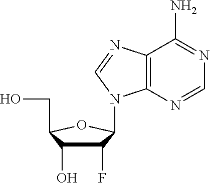

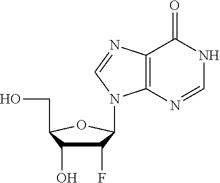

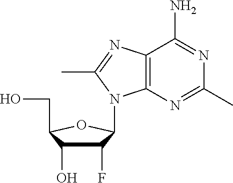

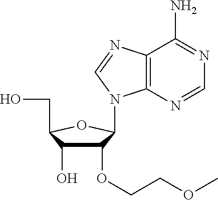

Exemplary Cytidine modifications include, but are not limited to:

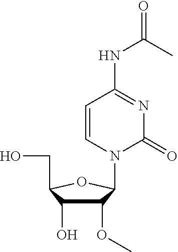

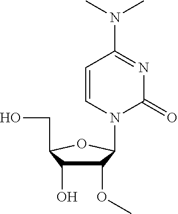

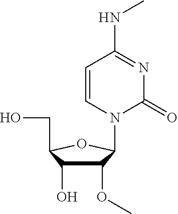

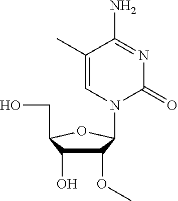

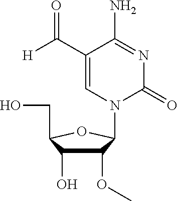

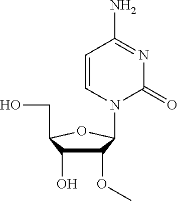

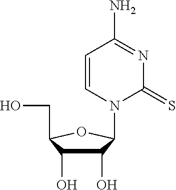

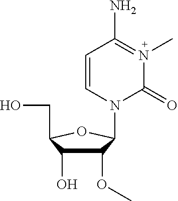

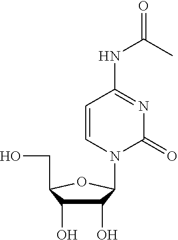

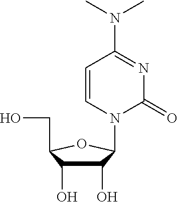

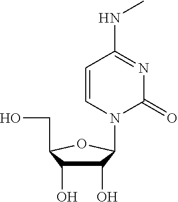

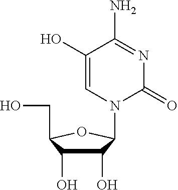

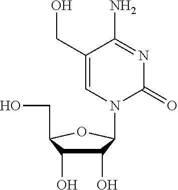

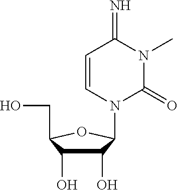

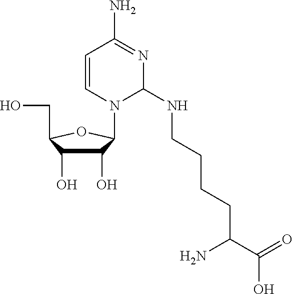

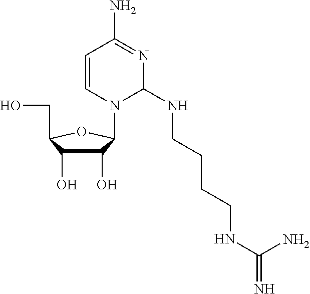

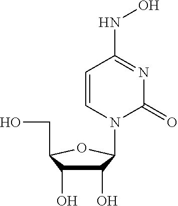

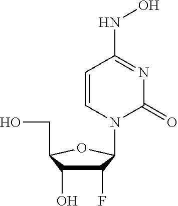

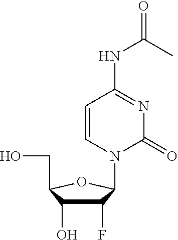

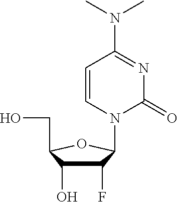

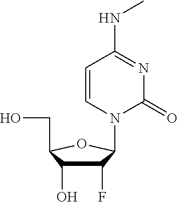

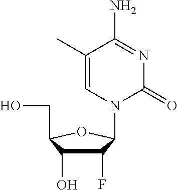

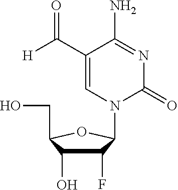

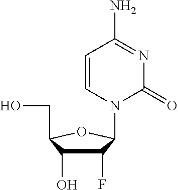

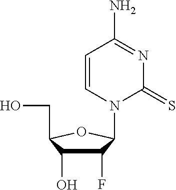

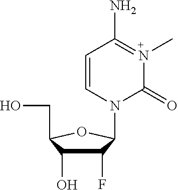

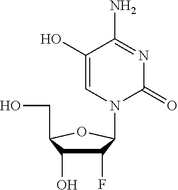

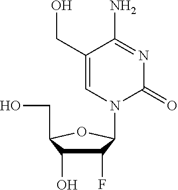

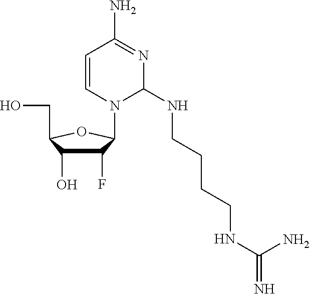

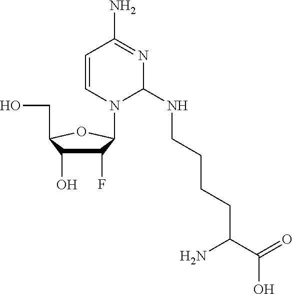

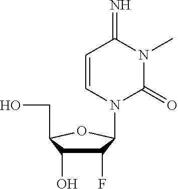

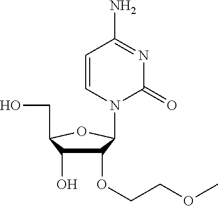

Exemplary Guanosine modifications include, but are not limited to:

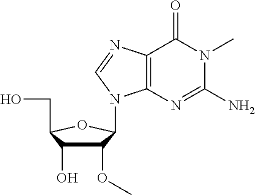

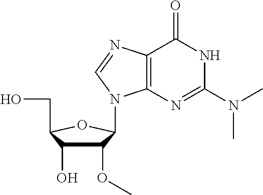

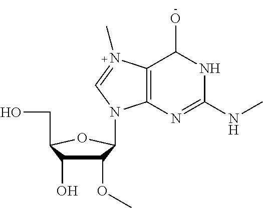

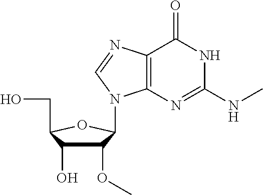

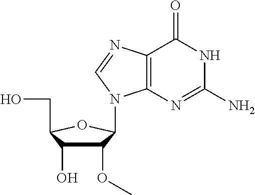

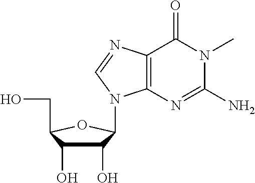

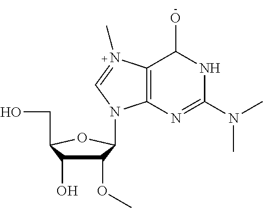

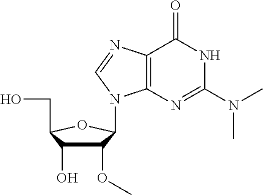

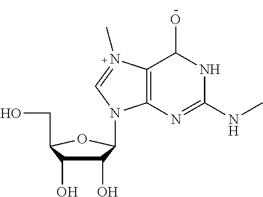

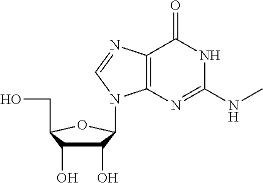

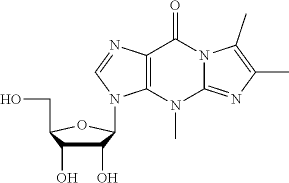

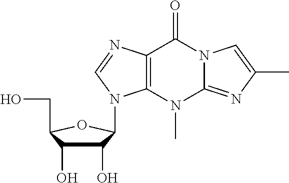

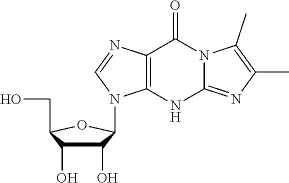

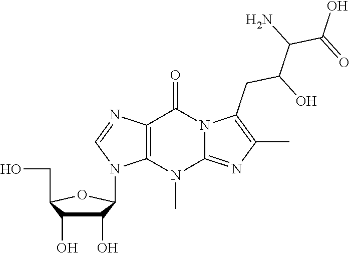

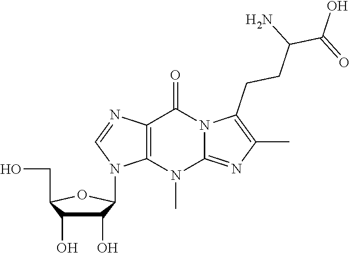

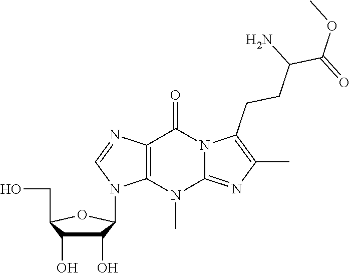

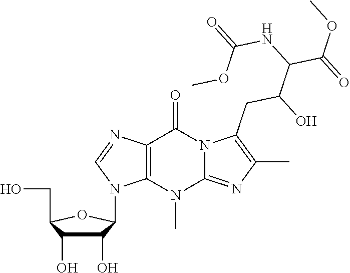

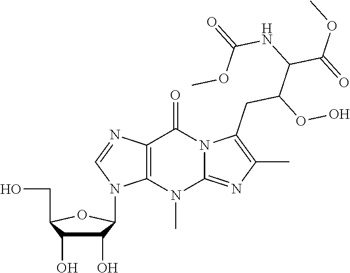

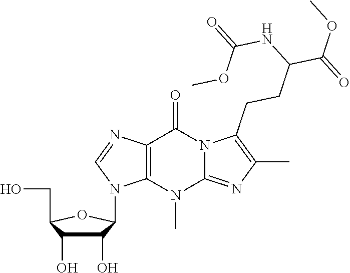

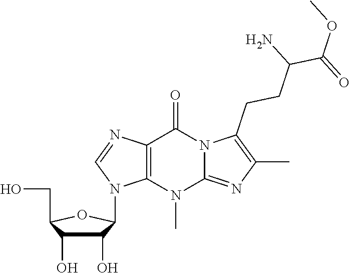

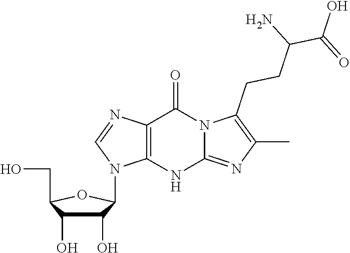

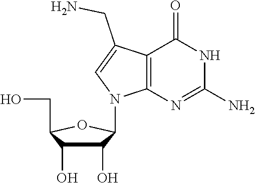

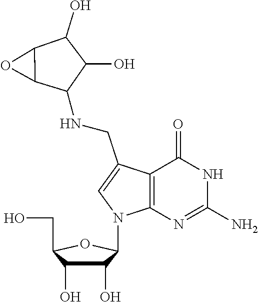

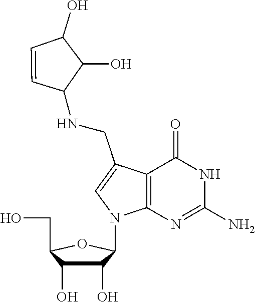

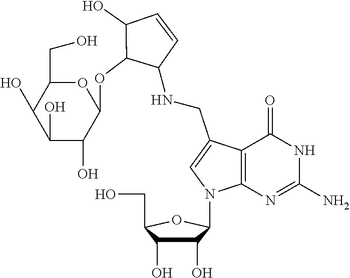

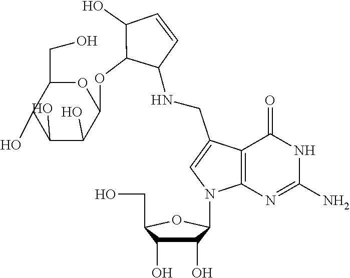

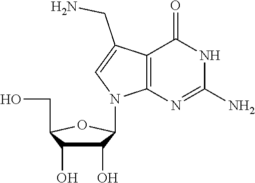

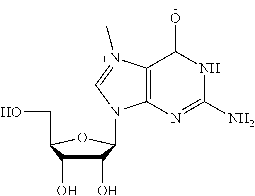

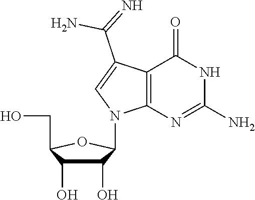

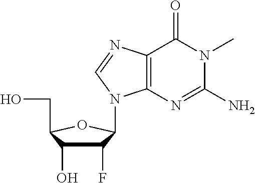

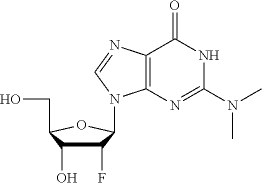

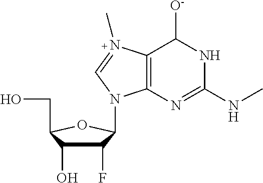

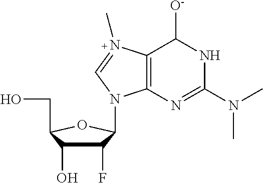

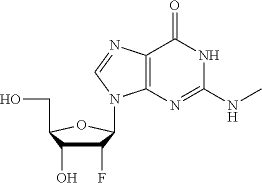

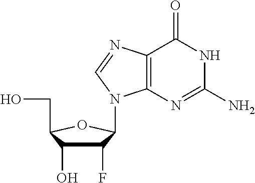

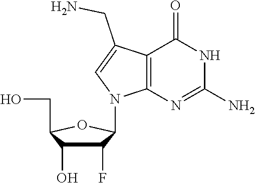

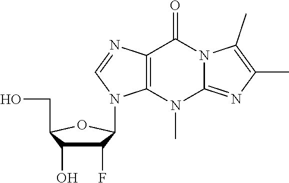

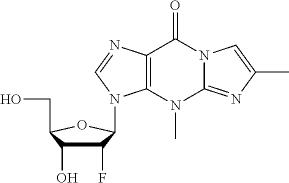

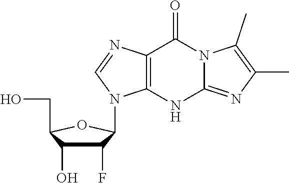

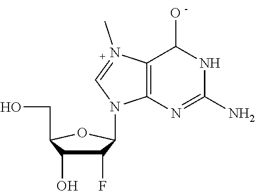

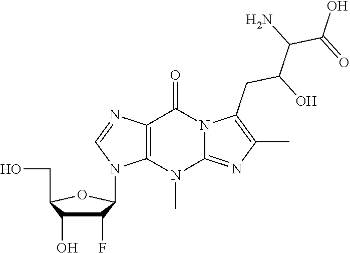

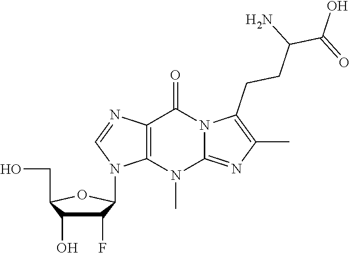

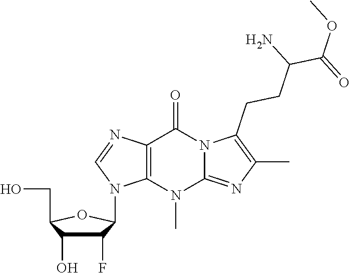

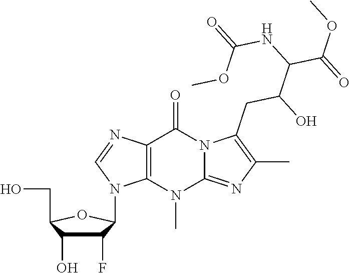

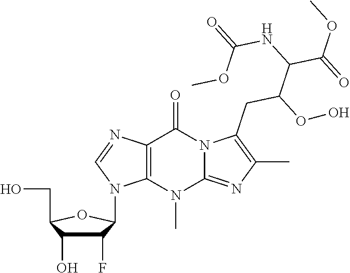

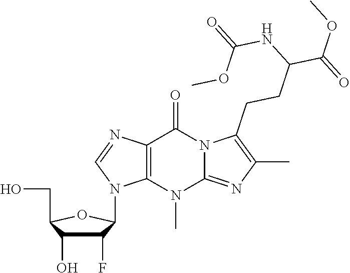

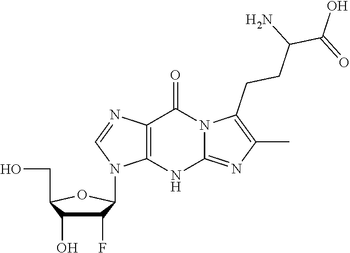

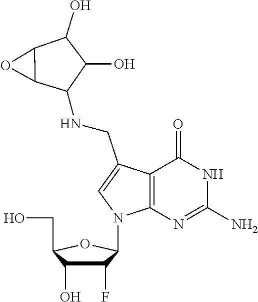

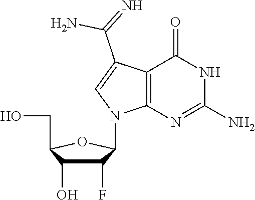

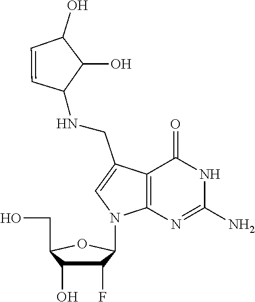

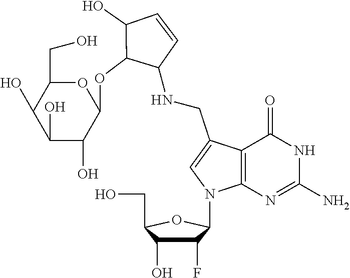

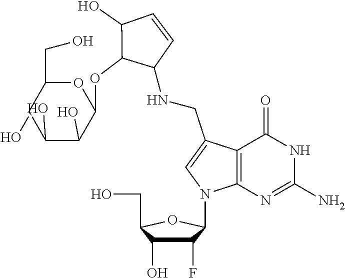

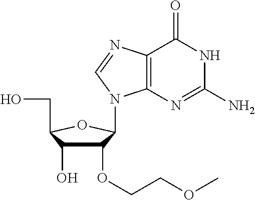

Exemplary embodiments of synthesis strategies for preparing nucleotide analogs of Uridine (scheme 1), Cytosine (scheme 2), Adenosine (scheme 3), and Guanosine (scheme 4).

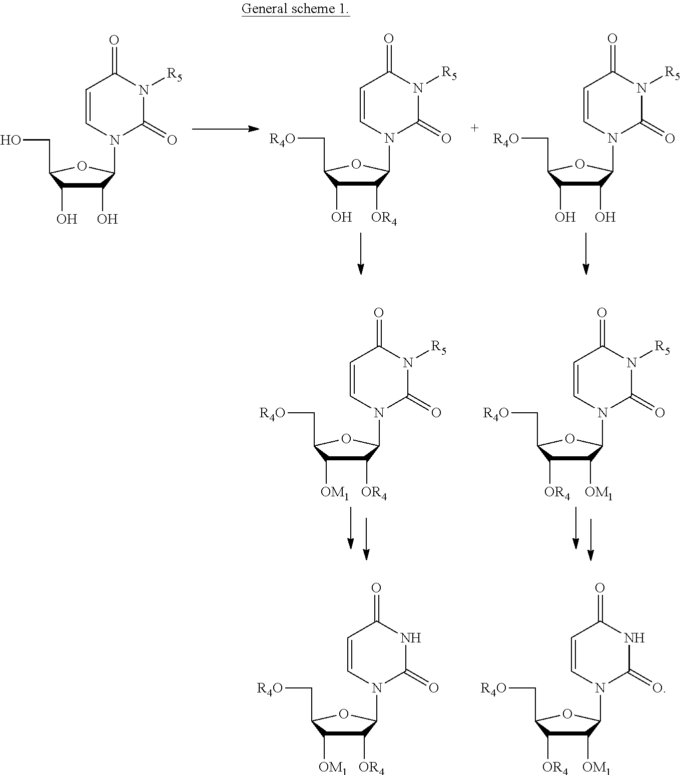

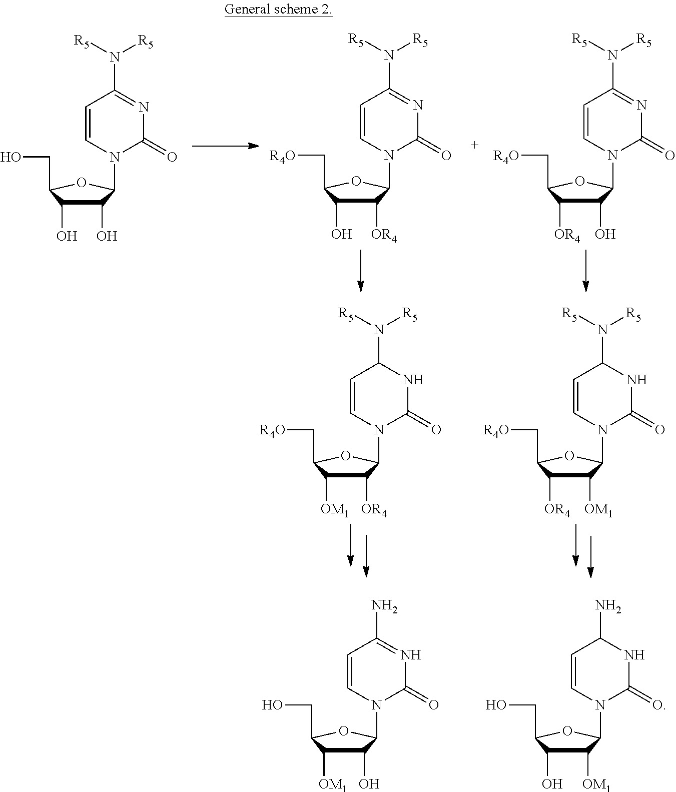

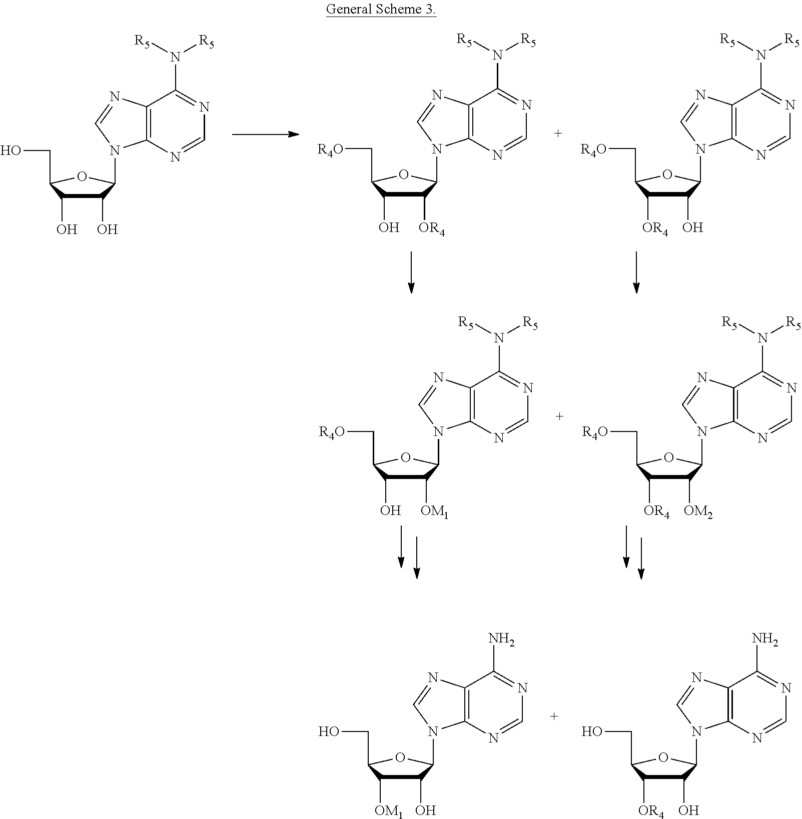

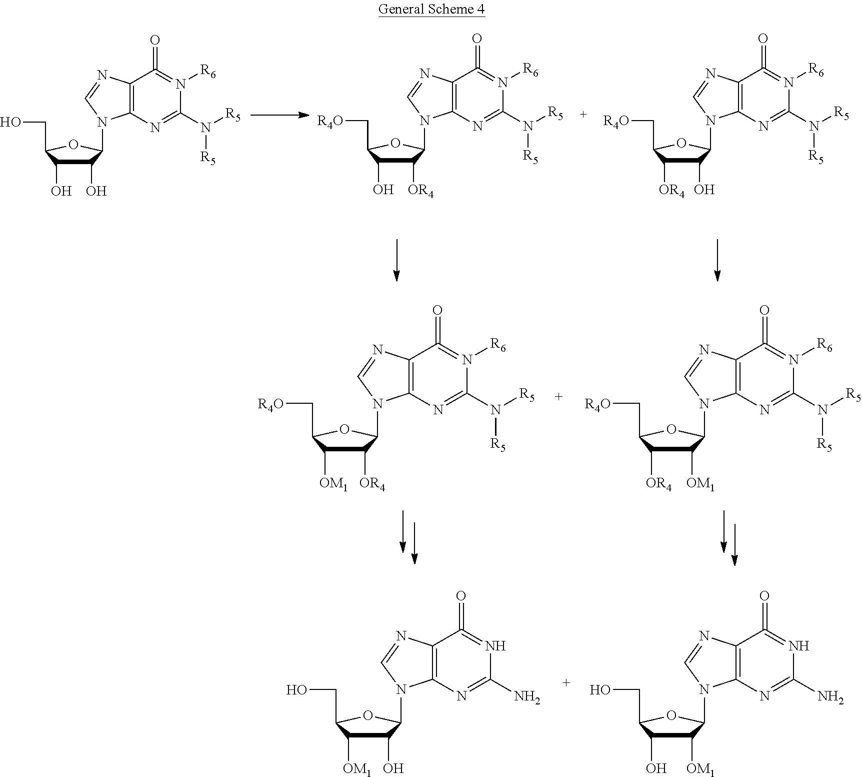

In various embodiments, Uridine analogs of the invention may include the following. While the below examples (and synthesis methods) are provided with respect to Uridine, the same or similar modifications will be understood to be also applied to other nucleotides (Uridine, Adenosine, Cytidine, and Guanosine) especially where such modifications are to the sugar.

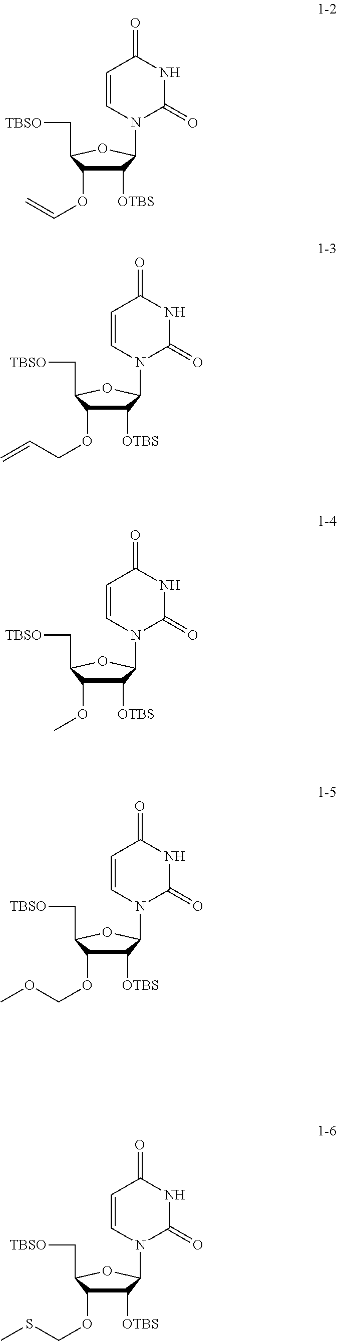

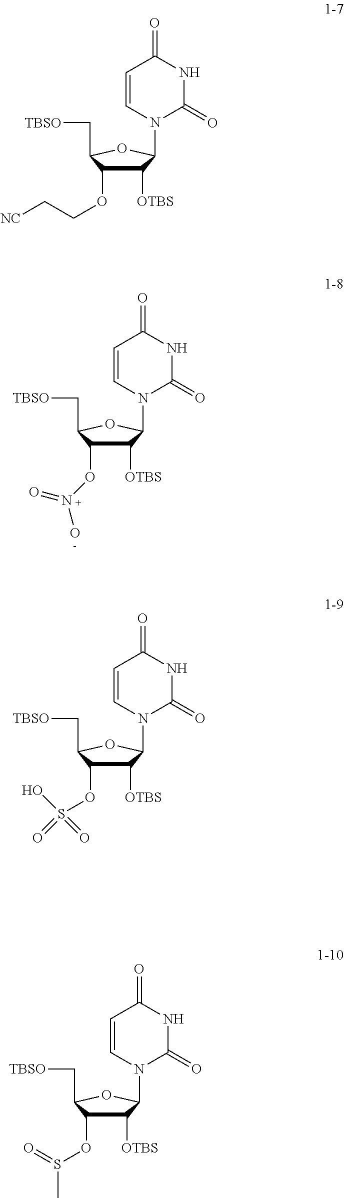

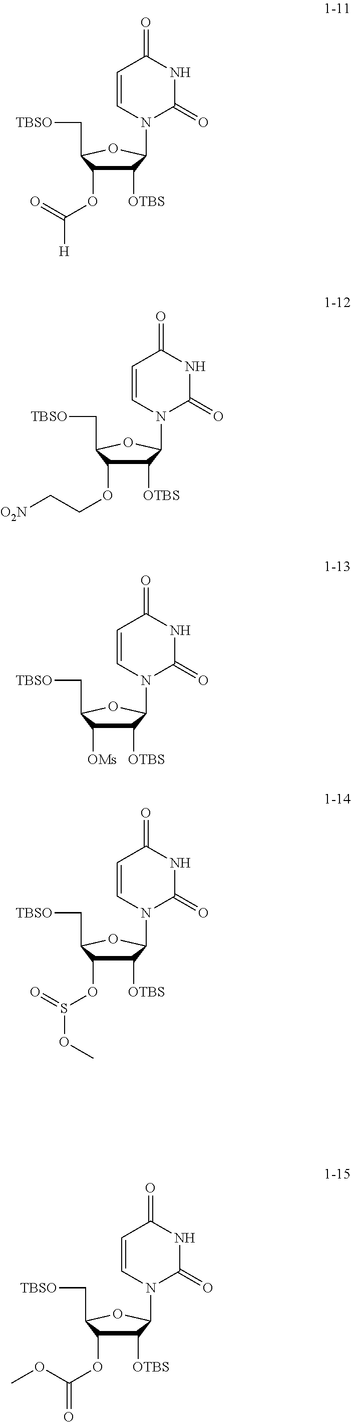

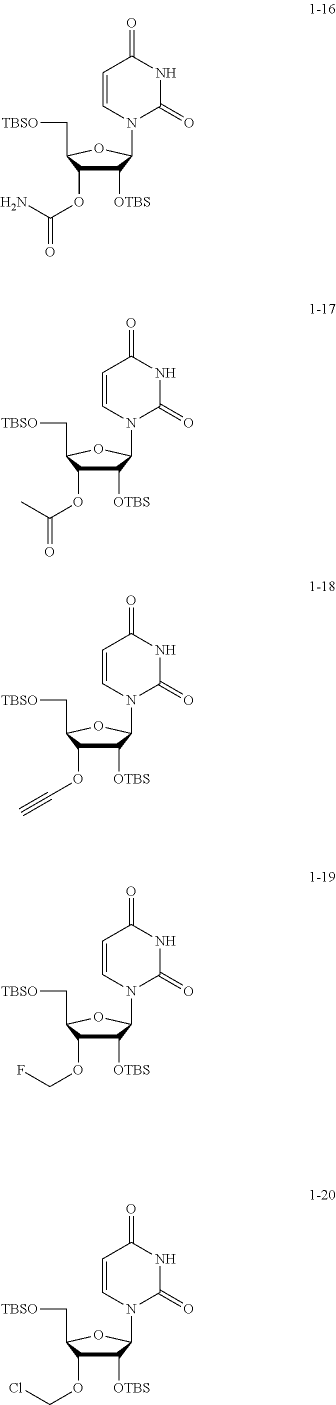

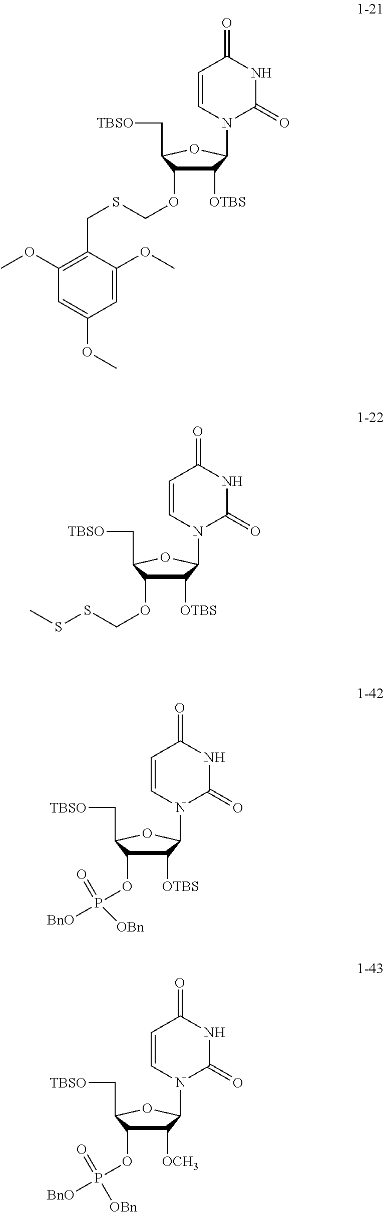

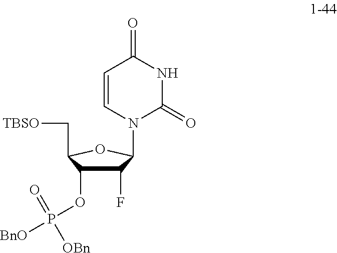

In various embodiments, the above Uridine analogs (1-1 through 1-22 and 1-42 through 1-44) may be deprotected (i.e., have their 2′-O-modification removed) to form the following analogs (1-23 through 1-41 and 1-45 through 1-47, respectively aside from 1-20 and 1-21). While the below examples (and deblocking methods) are provided with respect to Uridine, the same or similar modifications will be understood to be also applied to other nucleotides (Uridine, Adenosine, Cytidine, and Guanosine) especially where such modifications are to the sugar.

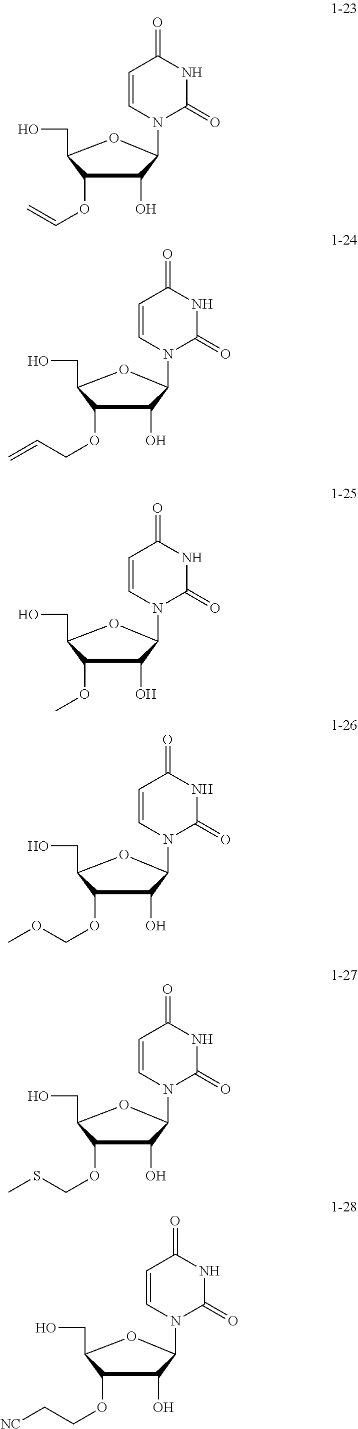

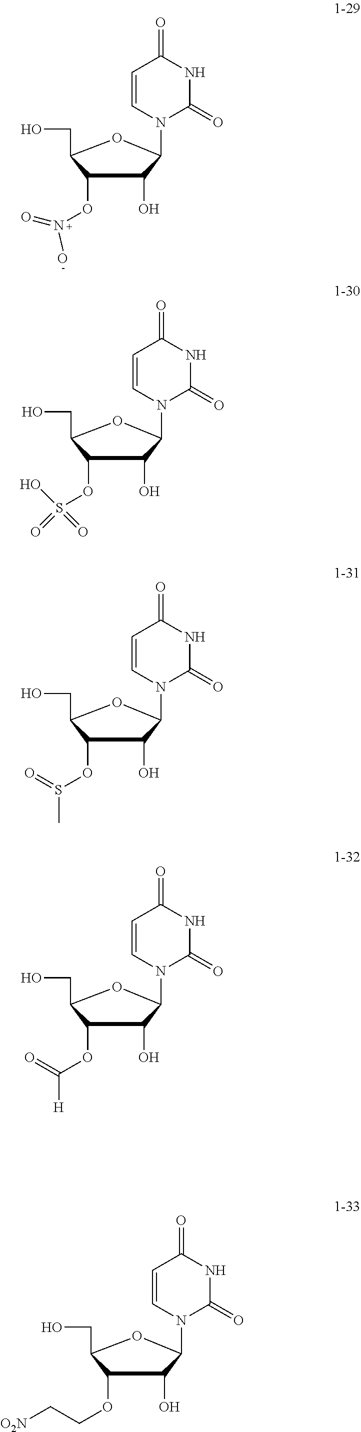

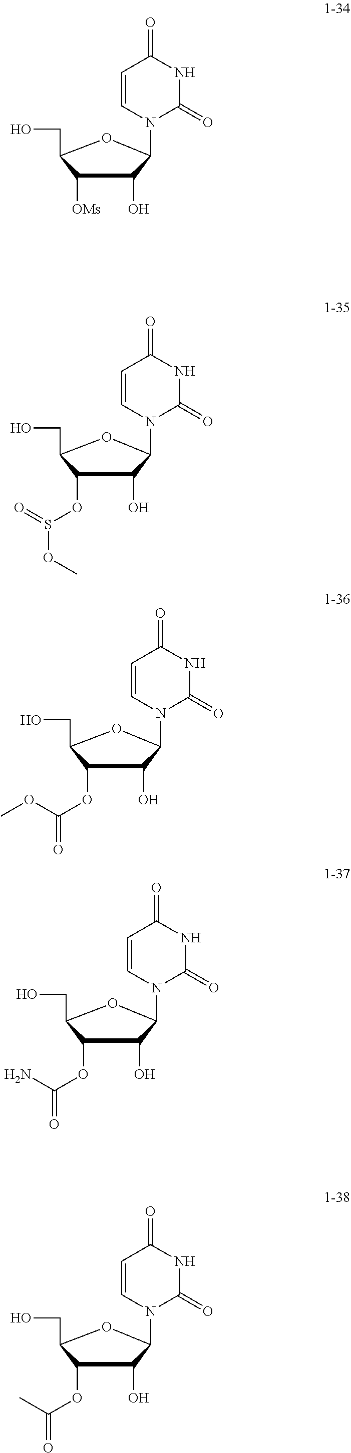

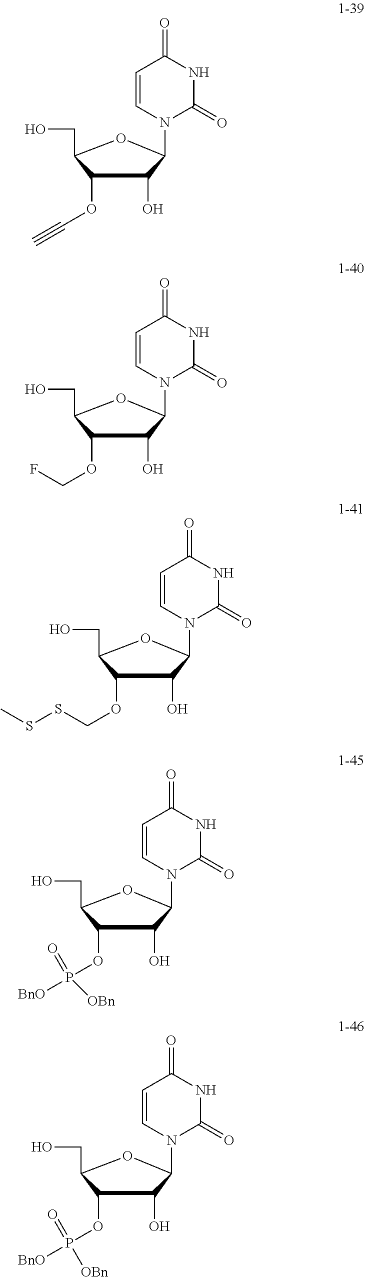

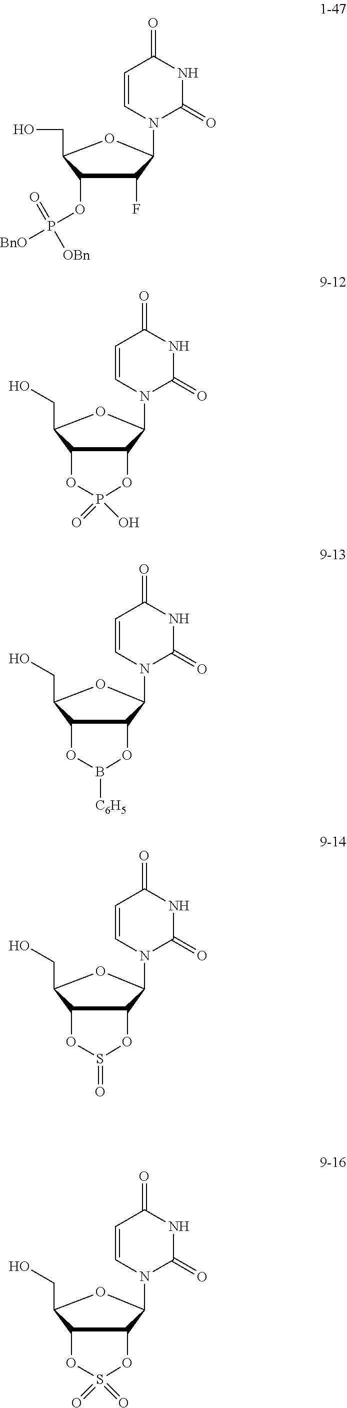

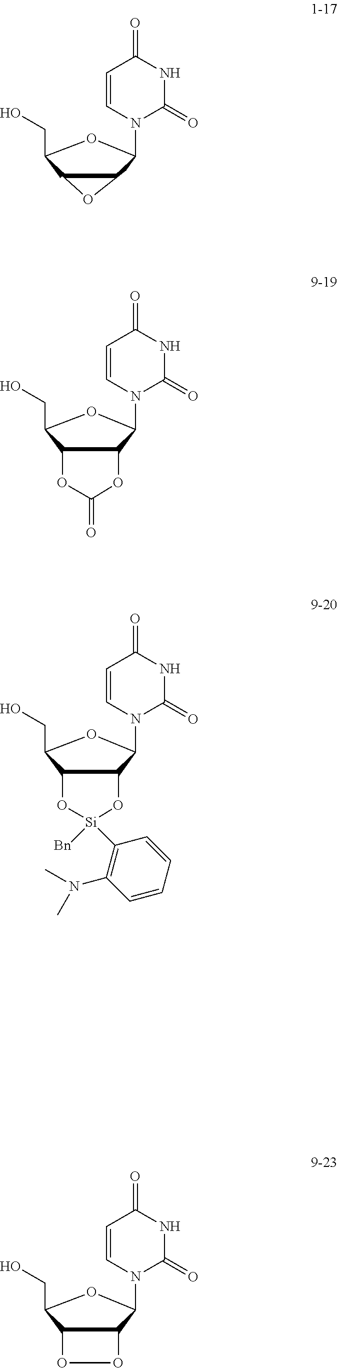

In various embodiments, Cytidine analogs of the invention may include the following:

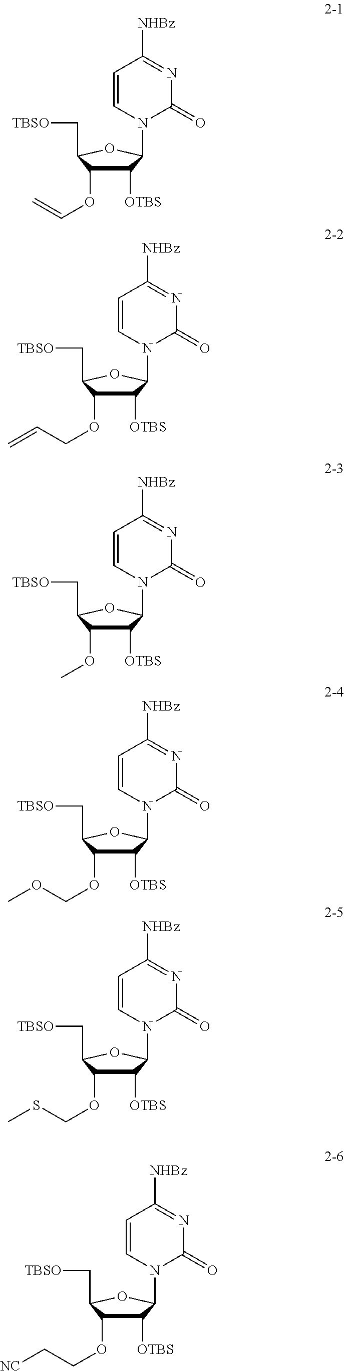

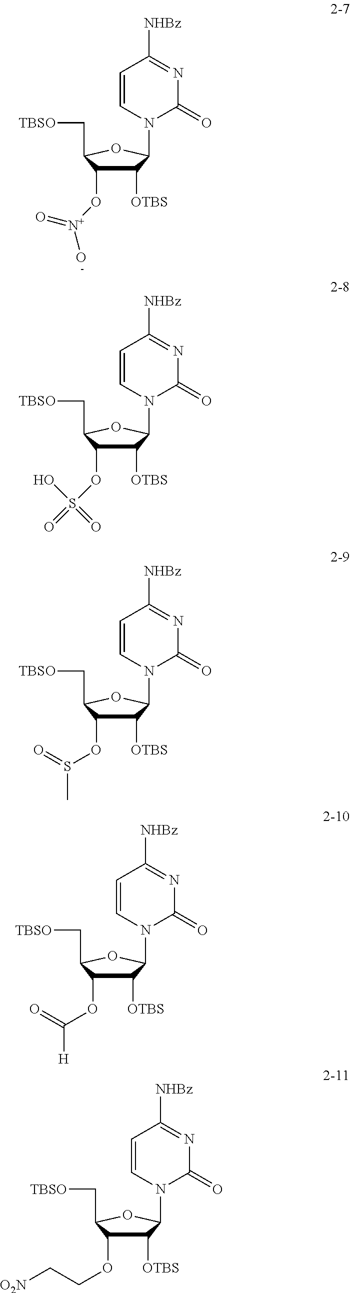

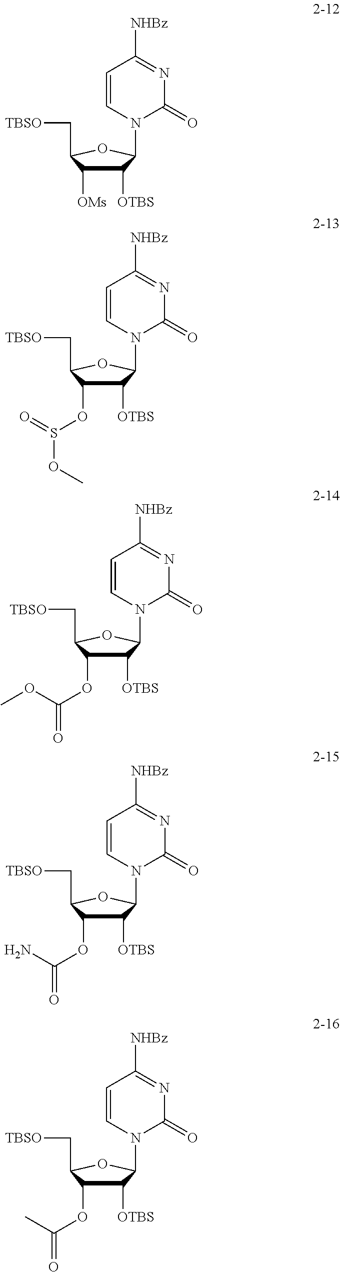

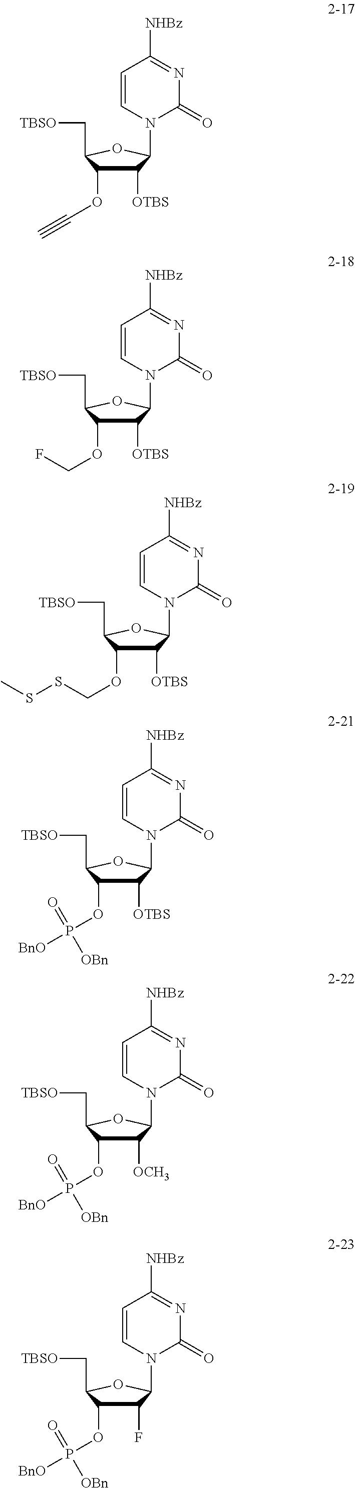

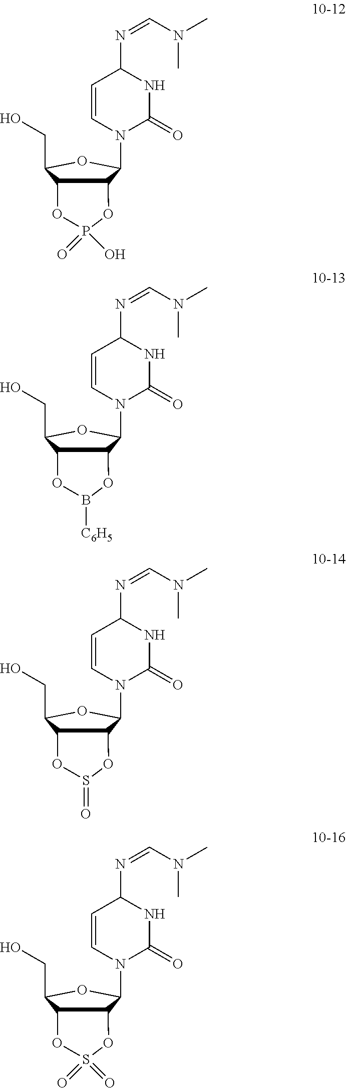

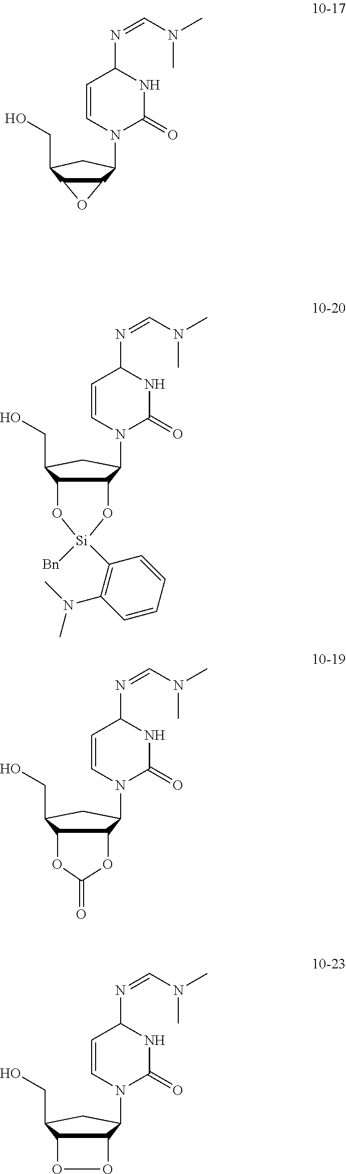

In various embodiments, Adenine analogs of the invention may include the following:

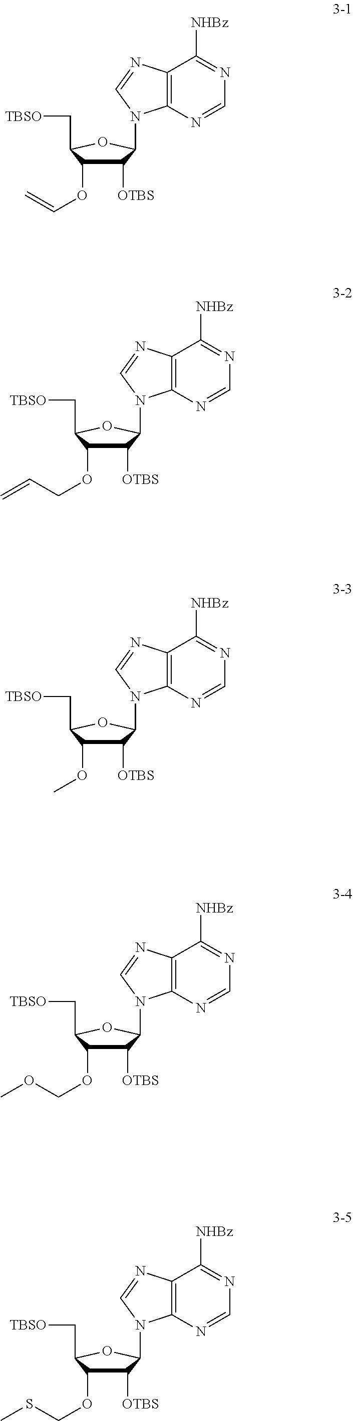

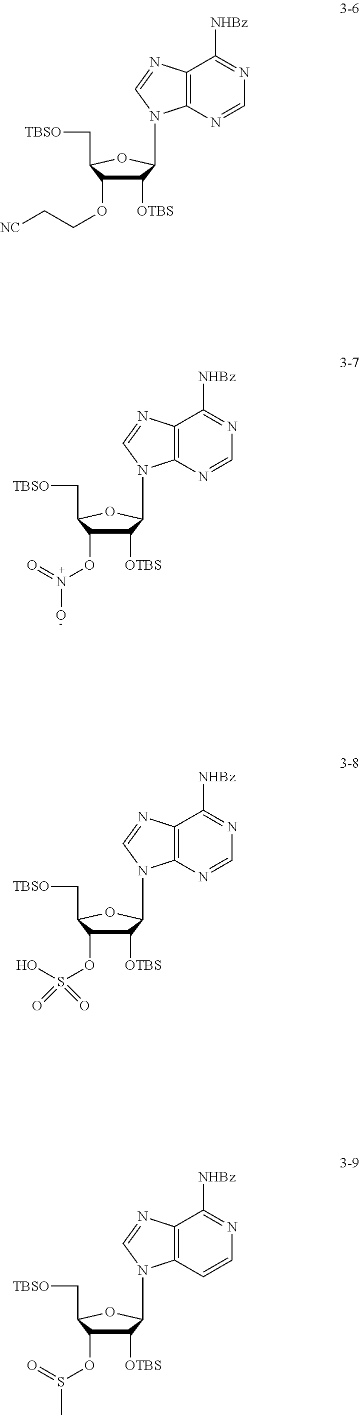

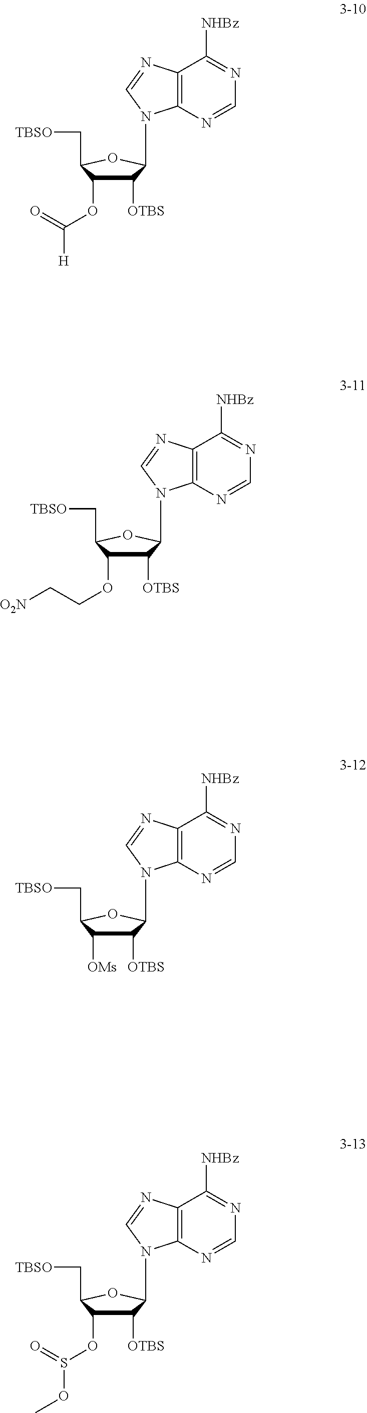

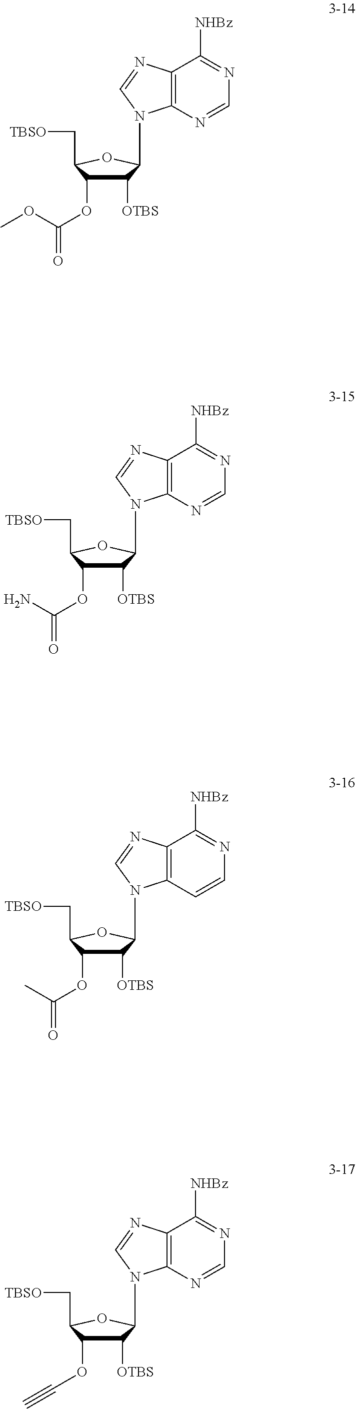

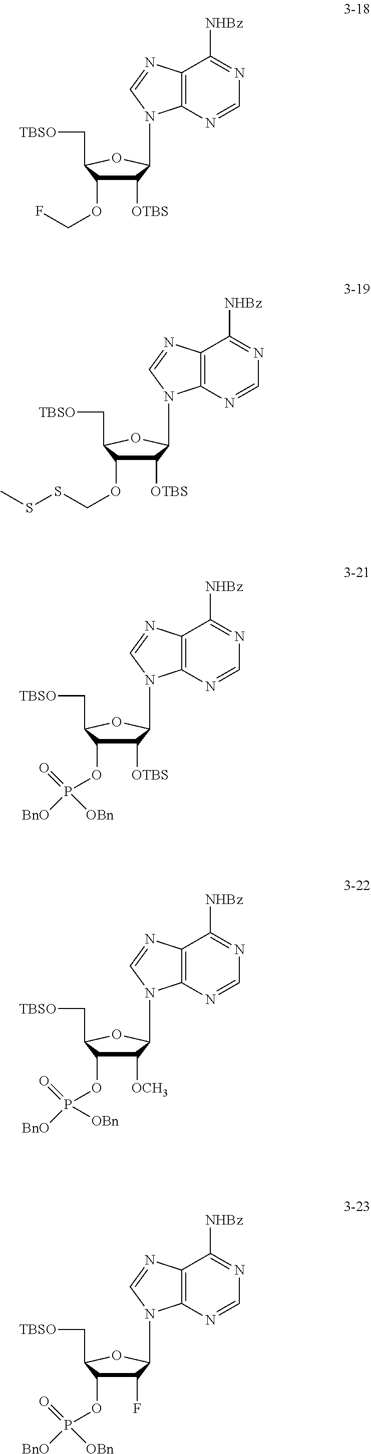

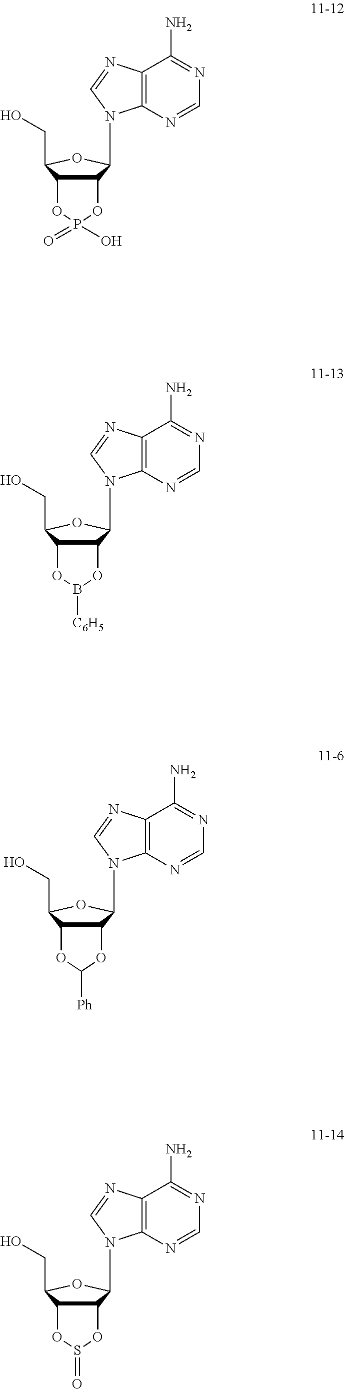

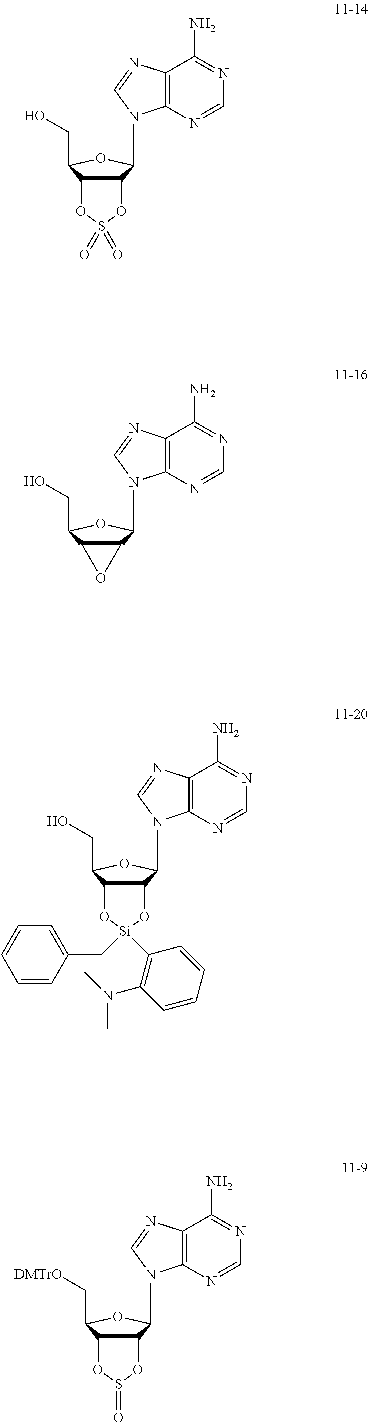

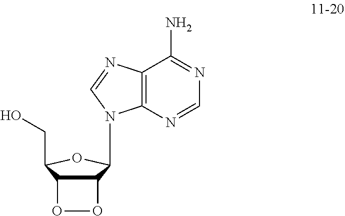

In various embodiments, Guanine analogs of the invention may include the following:

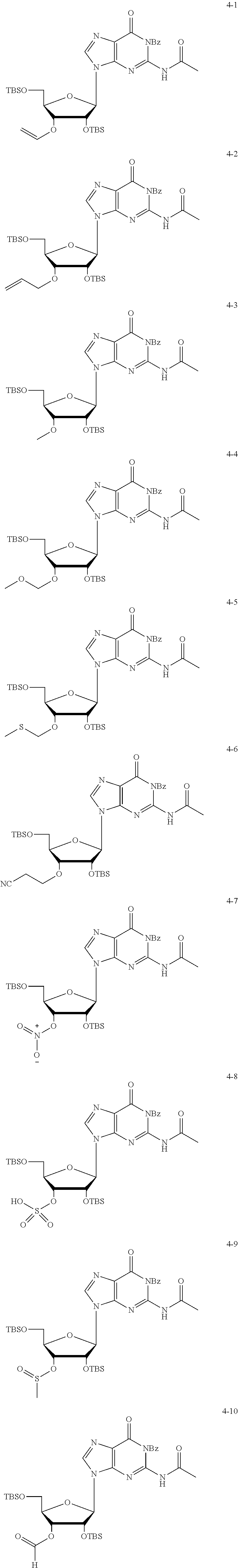

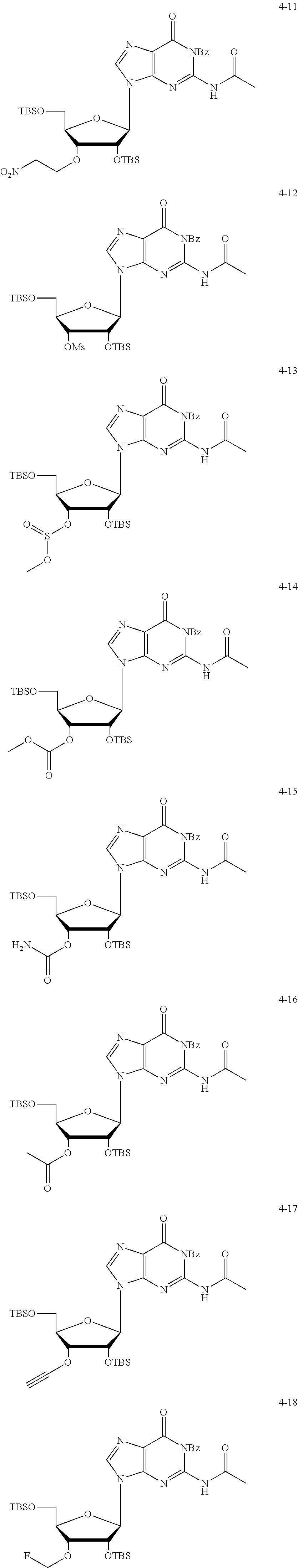

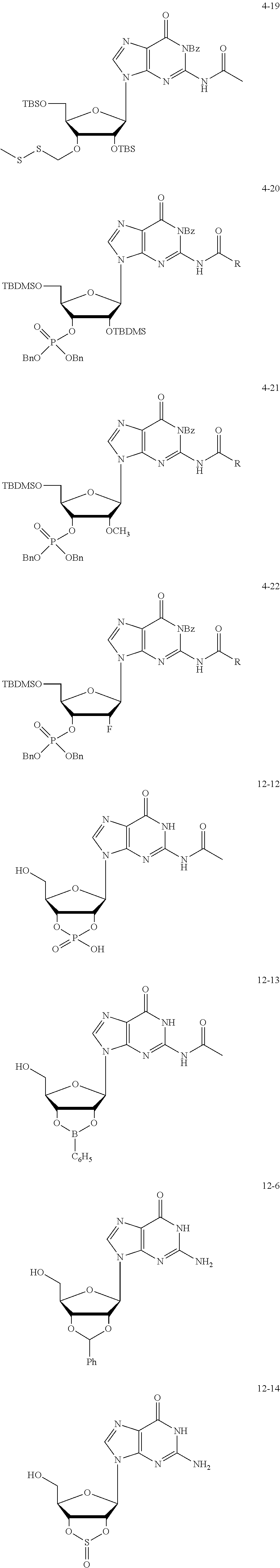

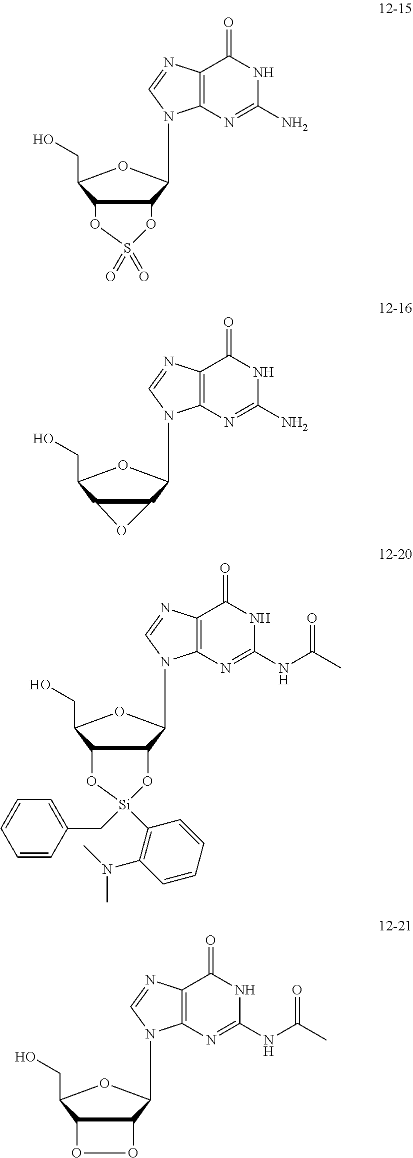

A variety of 3-O-modified dNTPs and NTPs may be used with the disclosed proteins for de novo synthesis. In some embodiments, the preferred removable 3′-O-blocking group is a 3′-O-amino, a 3′-O-allyl or a 3′-O-azidomethyl. In other embodiments, the removable 3′-O-blocking moiety is selected from the group consisting of O-phenoxyacetyl; O-methoxyacetyl; O-acetyl; O-(p-toluene)-sulfonate; O-phosphate; O-nitrate; O-[4-methoxy]-tetrahydrothiopyranyl; O-tetrahydrothiopyranyl; O-[5-methyl]-tetrahydrofuranyl; O-[2-methyl,4-methoxy]-tetrahydropyranyl; O-[5-methyl]-tetrahydropyranyl; and O-tetrahydrothiofuranyl (see U.S. Pat. No. 8,133,669). In other embodiments the removable blocking moiety is selected from the group consisting of esters, ethers, carbonitriles, phosphates, carbonates, carbamates, hydroxylamine, borates, nitrates, sugars, phosphoramide, phosphoramidates, phenylsulfenates, sulfates, sulfones and amino acids (see Metzker M L et al. Nuc Acids Res. 1994; 22(20):4259-67, U.S.P.N.

6,232,465; U.S.P.N. 7,414,116; and U.S.P.N. 7,279,563, all of which are incorporated by reference in their entireties).

In various embodiments, nucleotide analogs of the invention may have the following structure:

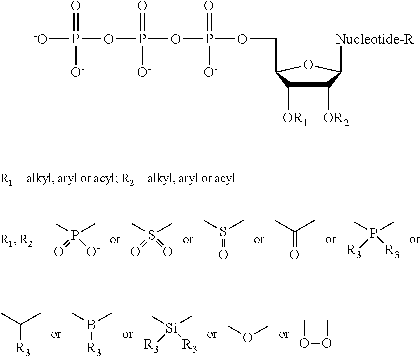

R1, R2 represents a 2′-O- or/and 3′-O-blocking group such as the ones discussed above. In certain embodiments, the 2′-O- or/and 3′-O-blocking group may be 3′-ONO₂, 3′-OCH₂CH₂CN, 3′-OCH₂N₃, 3′-OPO₃⁽⁻⁾, 3′-OCH₂SSCH₃, and 3′-ONHC(O)H. The Nucleotide-R group may be any of the nucleotide groups discussed above including various linkers and blocking groups or other modifications. In certain embodiments, R may be an H, an amide, a carbamate, or a urea. Any of those R groups may be further linked to a methyl, ethyl, propyl, isopropyl, isobutyl, pivaloyl, cyclohexyl, cyclopropyl, phenyl, naphthyl, anthracenyl, phenanthrenyl, pyrenyl, chrysenyl, pyridinyl, pyrimidinyl, pyrazinyl, indolyl, quinolinyl, isoquinolinyl, furanyl, thiophenyl, morpholinyl, piperidinyl, dioxanyl, tetrahydrofuranyl, or biotin.

The complete nucleotide-R group may comprise adenosine, cytidine, uridine, guanosine, an N6-modified adenosine, an N4-modified cytidine, an N3-modified uridine, an 06-modified guanosine, an N1-modified guanosine, or an N2-modified guanosine.

In various embodiments, the nucleotide analog may be only an N4-modified cytidine or an 06, N1, or N2-modified guanosine, each further comprising of a 3′-O-blocking group. In some embodiments, the nucleotide analog may be only an N4-modified cytidine or a, N1-modified uridine, each further comprising a 3′-O-blocking group, 2′-O-blocking group or 2′-O and/or 3′-O-blocking group.

For the synthesis of RNA polynucleotides, a polymerase like E. coli poly(A) polymerase can be used to catalyze the addition of ribonucleotides to the 3′ end of a ribonucleotide initiator. In other embodiments, E. coli poly(U) polymerase may be more suitable for use with the methods of the invention. Both E. coli poly(A) polymerase and E. coli poly(U) polymerase are available from New England Biolabs (Ipswich, MA). These enzymes may be used with 3′ unblocked reversible terminator ribonucleotide triphosphates (rNTPs) to synthesize RNA. In certain embodiments, RNA may be synthesized using 3′ blocked, 2′ blocked, or 2′,-3′ blocked rNTPs and poly(U) polymerase or poly(A) polymerase. The amino acid and nucleotide sequences for E. coli Poly(A) polymerase and E. coli Poly(U) polymerase are reproduced below. Modified E. coli Poly(A) polymerase or E. coli Poly(U) polymerase may be suitable for use with the methods of the invention. As an alternative, polymerase theta, or a derivative thereof, is used as the enzyme catalyzing base addition.

As discussed above, the inhibitor coupled to the nucleotide analog will cause the p o l y m e r a s e, e.g., TdT or pol theta, to not release from the polynucleotide or prevent other analogs from being incorporated into the growing chain. A charged moiety results in better inhibition, however, research suggests that the specific chemical nature of the inhibitor is not particularly important. For example, both phosphates and acidic peptides can be used to inhibit enzymatic activity. See, e.g., Bowers et al., Nature Methods, vol. 6, (2009) p. 593-95, and U.S. Pat. No. 8,071,755, both of which are incorporated herein by reference in their entireties. In some embodiments, the inhibitor will include single amino acids or dipeptides, like -(Asp)2, however the size and charge on the moiety can be adjusted, as needed, based upon experimentally determined rates of first nucleotide incorporation and second nucleotide incorporation. That is, other embodiments may use more or different charged amino acids or other biocompatible charged molecule.

In some embodiments, it may be advantageous to use a 3′ exonuclease to remove oligonucleotides that have not been properly terminated with an inhibitor prior to subsequent nucleotide analog addition. In particular, the inhibitor of the nucleotide analog can be chosen to inhibit the activity of polymerase and 3′ exonucleases, such that only properly terminated oligonucleotides would be built up. Using this quality control technique, the purity of the resulting oligonucleotide sequences would be improved. In some embodiments, use of such quality control measures can negate the need for post-synthesis purification.

### Solid State Synthesis

The methods of the invention can be practiced under a variety of reaction conditions, however the orderly construction and recovery of desired polynucleotides will, in most cases, require a solid support on which the polynucleotides can be extended. When used in conjunction with the NTP, linker, and inhibitor analogs discussed above, it is possible to construct specific polynucleotide sequences of RNA, by using, for example, TdT or pol theta in an aqueous environment. The polymerase can be used to affect the stepwise construction of custom polynucleotides by extending the polynucleotide sequence in a stepwise fashion. As discussed previously, the inhibitor group of each NTP analog causes the enzyme to stop with the addition of a nucleotide. After each nucleotide extension step, the reactants are washed away from the solid support prior to the removal of the inhibitor by cleaving the linker, and then new reactants are added, allowing the cycle to start anew.

In certain embodiments, an additional quality control step may be incorporated in which the oligonucleotide or polynucleotide is exposed to 3′ exonuclease after the polymerase-mediated nucleotide analog extension step and before inhibitor cleavage. A 3′ exonuclease degrades oligonucleotide or polynucleotide strands with an unblocked 3′ hydroxyl. An uncleaved inhibitor (e.g., a steric inhibitor) may physically block the 3′ exonuclease from degrading a strand to which an uncleaved nucleotide analog has been successfully incorporated. Such a quality control step degrades only the oligonucleotides or polynucleotides that have unsuccessfully incorporated the desired nucleotide analog in the prior addition step, thereby eliminating any errors in the finished synthesized sequence. After 3′ exonuclease exposure, the enzyme may be washed away before carrying on with the inhibitor cleavage step.

The 3′ exonuclease acts by shortening or completely degrading strands that have not successfully added the desired nucleotide analog. Strands that fail to be enzymatically extended at a given cycle will not have a terminal macromolecule-dNMP conjugate prior to the linker cleavage step. If a 3′-exonuclease is introduced at this stage, the full-length strand could be protected from degradation while “failure” strands will be shorted in length or potentially degraded completely to mononucleotide phosphates. The yield of long (>500 bases) synthetic RNA or DNA is dependent on highly efficient reactions occurring at each and every cycle; both the enzymatic extension and the deblocking/self-elimination steps must occur at near quantitative yields. The introduction of a 3′-exonuclease after the enzymatic extension step but before the macromolecule terminator cleavage step has a positive impact on the nascent strand purity if the extension efficiency is low (i.e., there are strands that are not extended and therefore possess a natural unmodified terminal nucleotide).

Conversely, a 3′-exonuclease step would have no impact on the quality of the synthesis if the deblocking/elimination step is less than quantitative because those strands would still be protected by the macromolecule terminator and fail to extend during the next extension step.

Thus, the actual improvement of the quality of the synthesis with the addition of a 3′-exonuclease step can only be experimentally determined and then an assessment made if it is worth the additional cost and cycle time.

Enzymes having 3′-5′ exonuclease activity include 3′-exonucleases as discussed above as well as polymerases having 3′-exonuclease. Exemplary enzymes having 3′-5′ exonuclease activity include RNase T, RNase PH, 3′hExo, RNase R, RNAase II, RNase D, Ccr4, Caf1/Pop2, PARN, Pan2, PNPase.

In certain embodiments, a nucleotide analog addition cycles may include exposing an oligonucleotide attached to a solid support to a nucleotide analog in the presence of a polymerase enzyme and in the absence of a nucleic acid template under conditions sufficient for incorporation of said analog into said oligonucleotide. The nucleotide analog can include a 3′-O-blocking group that prevents the polymerase from catalyzing addition of either a natural nucleotide or a nucleotide analog into said oligonucleotide until said blocking group is removed. After each nucleotide analog incorporation, the oligonucleotide may be exposed to a second nucleotide analog that does not confer resistance to exonuclease activity. The oligonucleotide may be then exposed to an enzyme having 3′-5′ exonuclease activity prior to removal of the 3′-blocking group. Strands where the desired 3′-O blocked nucleotide analog was not incorporated will instead have the second nucleotide analog incorporated or none at all (leaving an unmodified 3′hydroxyl susceptible to exonuclease activity). Accordingly, treatment with an exonuclease prior to removal of 3′-o blocking groups will result in the digestion of error strands in which the desired nucleotide analog was not incorporated. The second nucleotide analog is selected from the group consisting of a 2′,3′-dideoxy nucleotide and a 2′,3′-dehydro nucleotide.

The exonuclease treatment may occur after each nucleotide analog incorporation cycle or may be reserved and only performed after two or more nucleotide analog incorporations or after incorporation of the final nucleotide analog to complete the desired oligonucleotide sequence (but before removal of the final blocking group). In the case of sequence-terminating second nucleotide analogs, error strands in which a desired 3′-O blocked nucleotide analog was not successfully incorporated will be blocked from further extension by the strand-terminating second nucleotide and, therefore, will have no further 3′-O blocked nucleotide analogs incorporated. Accordingly, even if the exonuclease treatment is reserved for the final incorporation step, any error strands occurring along the way will remain susceptible to exonuclease activity.

In some embodiments, a nucleic acid initiator will include a 3′ moiety that will release the synthesized oligonucleotide when in the presence of a releasing agent. In some embodiments the initiator is a single-stranded oligonucleotide, such as a dimer, trimer, tetramer, pentamer, hexamer, septamer, or octomer. Because the 3′ moiety attached to the initiator is a substrate for the enzyme, e.g., a TdT, e.g., a modified TdT, or pol theta, the enzyme can add additional nucleotides or nucleotide analogs in a stepwise fashion. With each addition, the length of the synthesized oligonucleotide increases.

In some embodiments, the solid support and nucleic acid initiator including the 3′ moiety will be reusable, thereby allowing the initiator coupled to the solid support to be used again and again for the rapid synthesis of oligonucleotides. Solid supports suitable for use with the methods of the invention may include glass and silica supports, including beads, slides, pegs, or wells. In some embodiments, the support may be tethered to another structure, such as a polymer well plate or pipette tip. In some embodiments, the solid support may have additional magnetic properties, thus allowing the support to be manipulated or removed from a location using magnets. In other embodiments, the solid support may be a silica coated polymer, thereby allowing the formation of a variety of structural shapes that lend themselves to automated processing.

The selection of substrate material and covalent linkage chemistry between initiator and substrate is limited only by the ability of the construct to withstand the synthesis conditions without loss of initiator. Preferred embodiments utilize substrates and linkers of greater chemical stability than the initiator so that the overall construct stability is that of the attached oligonucleotide and not dependent on the substrate. In some embodiments, initiators may be synthesized in a 5′ to 3′-direction from a material presenting surface hydroxyl groups, though in preferred embodiments the initiator is instead grafted to the substrate so that density and initiator quality can be precisely controlled.

The covalent linkage between the initiator and the substrate may be any bond which does not compromise the stability of the construct. Preferred embodiments may utilize couplings between oligonucleotides containing either 5′-amine, 5′-hydroxyl, 5′-phosphate, 5′-sulfhydryl, or 5′-benzaldehyde groups and surfaces or resins containing formyl, chloromethyl, epoxide, amine, thiol, alkene, or terminal C—F bonds on the substrate.

In other embodiments, the cleavage site installation may be used to homogenize the enzymatic accessibility of the initiator oligonucleotides. Each enzyme used during the enzymatic synthesis cycle has its own steric footprint with potentially distinct optimal loadings and spacing from the surface. This can produce unexpected behavior in regard to the kinetics of stepwise addition and yield from the enzymatic cleavage process. In some embodiments, repeated cycles of cleavage site installation, cleavage, and regeneration may be conducted prior to the oligonucleotide synthesis so that the cleavage enzyme and the template-independent polymerase are accessing the same population of surface oligonucleotides.

Some embodiments may install multiple different cleavage sites throughout a strand during synthesis. Upon digestion, a complex library of the strands located between the cleavage sites may be released from the resin. Such sequences can then be further amplified and used for enzymatic assembly processes by a skilled artisan. This approach may be uniquely suited to parallel synthesis schemes in order to produce greater varieties of sequence fragments with relatively few distinct locations on a surface, or to avoid the synthesis of contiguous strands which risk secondary structure formation during synthesis.

### Synthesizers

To capitalize on the efficiency of the disclosed methods, an aqueous phase RNA synthesizer can be constructed to produce desired polynucleotides in substantial quantities. In one embodiment, a synthesizer will include four wells of the described rNTP analog reagents, i.e., rCTP, rATP, rGTP, and rUTP, as well as TdT at concentrations sufficient to effect polynucleotide growth. A plurality of initiating sequences can be attached to a solid support that is designed to be repeatedly dipped into each of the four wells, e.g., using a laboratory robot.

The robot could be additionally programmed to rinse the solid support in wash buffer between nucleotide additions, cleave the linking group by exposing the support to a deblocking agent, and wash the solid support a second time prior to moving the solid support to the well of the next desired nucleotide. With simple programming, it is possible to create useful amounts of desired nucleotide sequences in a matter of hours, and with substantial reductions of hazardous waste.

Ongoing synthesis under carefully controlled conditions will allow the synthesis of polynucleotides with lengths in the thousands of base pairs. Upon completion, the extension products are released from the solid support, whereupon they can be used as finished nucleotide sequences.

A highly parallel embodiment could consist of a series of initiator-solid supports on pegs in either 96 or 384 well formats that could be individually retracted or lowered so that the pegs can be indexed to contact the liquids in the wells in a controlled fashion. The synthesizer could thus consist of the randomly addressable peg device, four enzyme-rNTP analog reservoirs in the same format as the peg device (96 or 384 spacing), additional reagent reservoirs (washing, deblocking, etc.) in the same format as the peg device (96 or 384 spacing), and a transport mechanism (e.g., a laboratory robot) for moving the peg device from one reservoir to another in a user programmable, controlled, but random-access fashion. Care must be taken to avoid

contaminating each of the four enzyme-rNTP reservoirs since the contents are reused throughout the entire synthesis process to reduce the cost of each polynucleotide synthesis.

In alternative embodiments, the reagents (e.g., nucleotide analogs, enzymes, buffers) will be moved between solid supports, allowing the reagents to be recycled. For example, a system of reservoirs and pumps can move four different nucleotide analog solutions, wash buffers, and/or reducing agent solutions between one or more reactors in which the oligonucleotides will be formed. The reactors and pumps can be conventional, or the devices may be constructed using microfluidics. Because of the non-anhydrous (aqueous) nature of the process, no special care needs to be taken in the design of the hardware used to eliminate exposure to water. The synthesis process can take place with only precautions to control evaporative loss. A highly parallel embodiment could consist of a monolithic series of initiator-solid supports on pegs in either 96 or 384 well format that can be interfaced to a series of wells in the same matching format. Each well would actually be a reaction chamber that is fed by four enzyme-rNTP analog reservoirs and additional reagent reservoirs (washing, deblocking, etc.) with appropriate valves. Provisions would be made in the fluidics logic to recover the enzyme-rNTP reactants in a pristine fashion after each extension reaction since they are reused throughout the entire synthesis process to reduce the cost of each polynucleotide synthesis. In other embodiments, a system of pipetting tips could be used to add and remove reagents.

In certain aspects, polynucleotides may be synthesized using microfluidic devices and/or inkjet printing technology. Microfluidic channels, including regulators, couple reservoirs to a reaction chamber and an outlet channel, including a regulator can evacuate waste from the reaction chamber. Microfluidic devices for polynucleotide synthesis may include, for example, channels, reservoirs, and/or regulators. Polynucleotide synthesis may occur in a microfluidic reaction chamber which may include a number of anchored synthesized nucleotide initiators which may include beads or other substrates anchored or bound to an interior surface of the reaction chamber and capable of releasably bonding a NTP analog or polynucleotide initiator. The reaction chamber may include at least one intake and one outlet channel so that reagents may be added and removed to the reaction chamber. The microfluidic device may include a reservoir for each respective NTP analog. Each of these NTP analog reservoirs may also include an appropriate amount of pol theta or any other enzyme which elongates DNA or RNA strands without template direction. Additional reservoirs may contain reagents for linker/inhibitor cleavage and washing. These reservoirs can be coupled to the reaction chamber via separate channels and reagent flow through each channel into the reaction chamber may be individually regulated through the use of gates, valves, pressure regulators, or other means. Flow out of the reaction chamber, through the outlet channel, may be similarly regulated.

In certain instances, reagents may be recycled, particularly the NTP analog-enzyme reagents. Reagents may be drawn back into their respective reservoirs from the reaction chamber via the same channels through which they entered by inducing reverse flow using gates, valves, pressure regulators or other means. Alternatively, reagents may be returned from the reaction chamber to their respective reservoirs via independent return channels. The microfluidic device may include a controller capable of operating the gates, valves, pressure, or other regulators described above.

An exemplary microfluidic polynucleotide synthesis reaction may include flowing a desired enzyme-NTP analog reagent into the reaction chamber; after a set amount of time, removing the enzyme-NTP analog reagent from the reaction chamber via an outlet channel or a return channel; flowing a wash reagent into the reaction chamber; removing the wash reagent from the reaction chamber through an outlet channel; flowing a de-blocking or cleavage reagent into the reaction chamber; removing the de-blocking or cleavage reagent from the reaction chamber via an outlet channel or a return channel; flowing a wash reagent into the reaction chamber; removing the wash reagent from the reaction chamber through an outlet channel; flowing the enzyme-NTP analog reagent including the next NTP in the desired sequence to be synthesized into the reaction chamber; and repeating until the desired polynucleotide has been synthesized. After the desired polynucleotide has been synthesized, it may be released from the reaction chamber anchor or substrate and collected via an outlet channel or other means.

In certain aspects, reagents and compounds, including NTP analogs, Pol Theta, TdT and/or other enzymes, and reagents for linker/inhibitor cleavage and/or washing may be deposited into a reaction chamber using inkjet printing technology or piezoelectric drop-on-demand (DOD) inkjet printing technology. Inkjet printing technology can be used to form droplets, which can be deposited, through the air, into a reaction chamber. Reagent droplets may have volumes in the picoliter to nanoliter scale. Droplets may be introduced using inkjet printing technology at a variety of frequencies including 1 Hz, 10; Hz, 100 Hz, 1 kHz, 2 kHz, and 2.5 kHz. Various reagents may be stored in separate reservoirs within the inkjet printing device and the inkjet printing device may deliver droplets of various reagents to various discrete locations including, for example, different reaction chambers or wells within a chip. In certain embodiments, inkjet and microfluidic technologies may be combined wherein certain reagents and compounds are delivered to the reaction chamber via inkjet printing technology while others are delivered via microfluidic channels or tubes. An inkjet printing device may be controlled by a computing device comprising at least a non-transitory, tangible memory coupled to a processor. The computing device may be operable to receive input from an input device including, for example, a touch screen, mouse, or keyboard and to control when and where the inkjet printing device deposits a droplet of reagent, the reagent it deposits, and/or the amount of reagent deposited.

In certain instances, a desired polynucleotide sequence may be entered into the computing device through an input device wherein the computing device is operable to perform the necessary reactions to produce the desired polynucleotide sequence by sequentially depositing the appropriate NTP analog, enzyme, cleavage reagent, and washing reagent, in the appropriate order as described above.

After synthesis, the released extension products can be analyzed by high resolution PAGE to determine if the initiators have been extended by the anticipated number of bases compared to controls. A portion of the recovered synthetic RNA may also be sequenced to determine if the synthesized polynucleotides are of the anticipated sequence.

Because the synthesizers are relatively simple and do not require the toxic components needed for phosphoramidite synthesis, synthesizers of the invention will be widely accessible for research institutions, biotechnology companies, and hospitals. Additionally, the ability to reuse/recycle reagents will reduce the waste produced and help reduce the costs of consumables.

### EXAMPLES

### Example 1: The Uridine Analogs (1-1 Through 1-22 Above) were Prepared Using the Following Methods

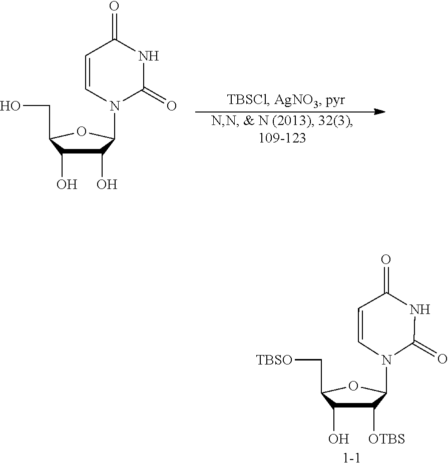

### 1-((2R,3R,4R,5R)-3-((tert-butyldimethylsilyl)oxy)-5-(((tert-butyldimethylsilyl)oxy)methyl)-4-hydroxytetrahydrofuran-2-yl)pyrimidine-2,4(1H,3H)-dione

To a suspension of 1-((2R,3R,4S,5R)-3,4-dihydroxy-5-(hydroxymethyl)tetrahydrofuran-2-yl)pyrimidine-2,4(1H,3H)-dione (1 equiv.) in dry THF at rt under Ar were added pyridine (5 equiv.) and silver nitrate (2.2 equiv). The reaction was stirred for 10 min. To the reaction mixture was then added TBSCl (2.2 equiv.) and allowed to stir overnight. The reaction was quenched with ethanol (10 mL) and the mixture was vacuum filtered over celite. The filtrate was concentrated, re-dissolved in EtOAc and sequentially washed with water (25 mL×1) and brine (25 mL×1). The organic phase was dried over Na₂SO₄, filtered, and purified by chromatography (0-5% MeOH in DCM) to afford 1-((2R,3R,4R,5R)-3-((tert-butyldimethylsilyl)oxy)-5-(((tert-butyldimethylsilyl)oxy)methyl)-4-hydroxytetrahydrofuran-2-yl)pyrimidine-2,4(1H,3H)-dione.

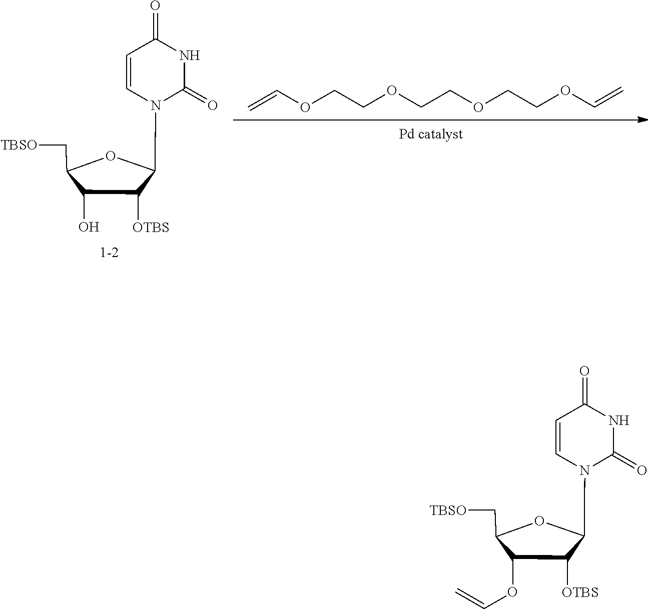

### 1-((2R,3R,4R,5R)-3-((tert-butyldimethylsilyl)oxy)-5-(((tert-butyldimethylsilyl)oxy)methyl)-4-(vinyloxy)tetrahydrofuran-2-yl)pyrimidine-2,4(1H,3H)-dione

To a suspension of 1-((2R,3R,4R,5R)-3-((tert-butyldimethylsilyl)oxy)-5-(((tert-butyldimethylsilyl)oxy)methyl)-4-hydroxytetrahydrofuran-2-yl)pyrimidine-2,4(1H,3H)-dione (1 equiv.) in triethylene glycol divinyl ether and acetonitrile (1:1, v/v) was added diacetato (1,10-phenanthroline)palladium(II) and allowed to stir at 80° C. open to the air overnight. The contents were concentrated via rotovap, dissolved in DCM, and subjected directly to column chromatography (0-5% MeOH in DCM) to afford 1-((2R,3R,4R,5R)-3-((tert-butyldimethylsilyl)oxy)-5-(((tert-butyldimethylsilyl)oxy)methyl)-4-(vinyloxy)tetrahydrofuran-2-yl)pyrimidine-2,4(1H,3H)-dione.

[Reference: J. Org. Chem. 2007, 72, 4250-4253]

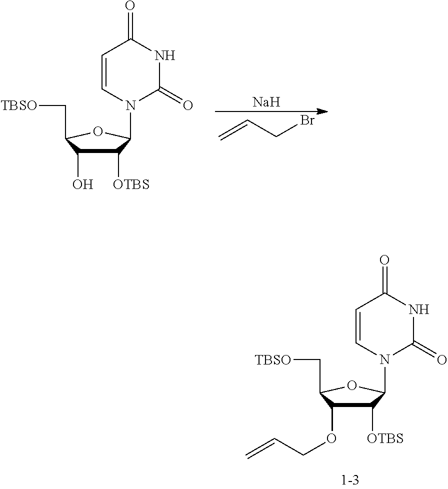

### 1-((2R,3R,4R,5R)-4-(allyloxy)-3-((tert-butyldimethylsilyl)oxy)-5-(((tert-butyldimethylsilyl)oxy)methyl)tetrahydrofuran-2-yl)pyrimidine-2,4(1H,3H)-dione

To a solution of 1-((2R,3R,4R,5R)-3-((tert-butyldimethylsilyl)oxy)-5-(((tert-butyldimethylsilyl)oxy)methyl)-4-hydroxytetrahydrofuran-2-yl)pyrimidine-2,4(1H,3H)-dione (1 equiv.) in THF at rt under Ar was added sodium hydride (60%, 1.5 equiv.) and stirred for 30 min. Allyl bromide (1 equiv.) was added to the mixture and stirred for 1 h. The reaction mixture was diluted with EtOAc and washed with water. The organic phase was dried over Na₂SO₄, filtered, and purified by chromatography (0-5% MeOH in DCM) to afford 1-((2R,3R,4R,5R)-4-(allyloxy)-3-((tert-butyldimethylsilyl)oxy)-5-(((tert-butyldimethylsilyl)oxy)methyl)tetrahydrofuran-2-yl)pyrimidine-2,4(1H,3H)-dione.

[Reference for selective O-allylation: Carbohydrate Research 343 (2008) 1490-1495]

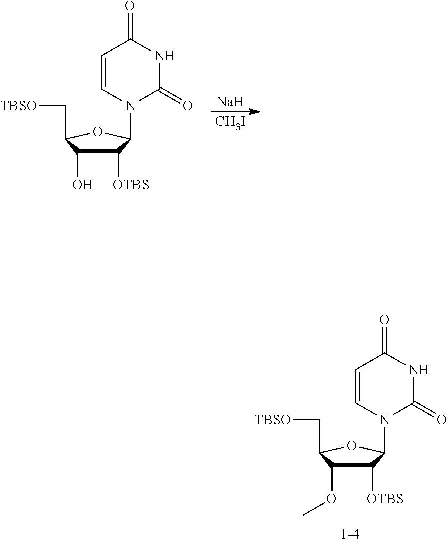

### 1-((2R,3R,4R,5R)-3-((tert-butyldimethylsilyl)oxy)-5-(((tert-butyldimethylsilyl)oxy)methyl)-4-methoxytetrahydrofuran-2-yl)pyrimidine-2,4(1H,3H)-dione

To a solution of 1-((2R,3R,4R,5R)-3-((tert-butyldimethylsilyl)oxy)-5-(((tert-butyldimethylsilyl)oxy)methyl)-4-hydroxytetrahydrofuran-2-yl)pyrimidine-2,4(1H,3H)-dione (1 equiv.) in THE at rt under Ar was added NaH (60%, 1.5 equiv.) and stirred for 30 min. Methyl bromide (1 equiv.) was added to the mixture and stirred for 1 h. The reaction mixture was diluted with EtOAc and washed with water. The organic phase was dried over Na₂SO₄, filtered, and purified by chromatography (0-5% MeOH in DCM) to afford 1-((2R,3R,4R,5R)-3-((tert-butyldimethylsilyl)oxy)-5-(((tert-butyldimethylsilyl)oxy)methyl)-4-methoxytetrahydrofuran-2-yl)pyrimidine-2,4(1H,3H)-dione.

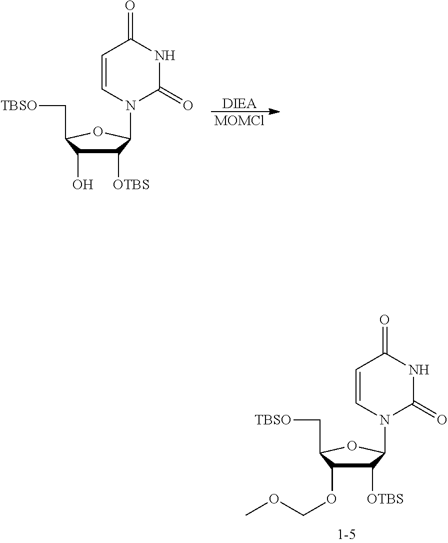

### 1-((2R,3R,4R,5R)-3-((tert-butyldimethylsilyl)oxy)-5-(((tert-butyldimethylsilyl)oxy)methyl)-4-(methoxymethoxy)tetrahydrofuran-2-yl)pyrimidine-2,4(1H,3H)-dione

To a solution of 1-((2R,3R,4R,5R)-3-((tert-butyldimethylsilyl)oxy)-5-(((tert-butyldimethylsilyl)oxy)methyl)-4-hydroxytetrahydrofuran-2-yl)pyrimidine-2,4(1H,3H)-dione in DIEA at 0° C. under Ar was added chloromethyl methyl ether (4 equiv.) and stirred for 1 h. The reaction mixture was quenched with water and aqueous phase was washed with DCM. The combined organic phase was dried over Na₂SO₄, filtered, and purified by chromatography (0-5% MeOH in DCM) to afford 1-((2R,3R,4R,5R)-3-((tert-butyldimethylsilyl)oxy)-5-(((tert-butyldimethylsilyl)oxy)methyl)-4-(methoxymethoxy)tetrahydrofuran-2-yl)pyrimidine-2,4(1H,3H)-dione.

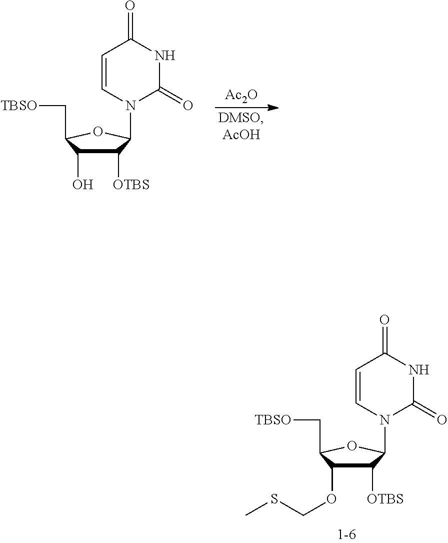

### 1-((2R,3R,4R,5R)-3-((tert-butyldimethylsilyl)oxy)-5-(((tert-butyldimethylsilyl)oxy)methyl)-4-((methylthio)methoxy)tetrahydrofuran-2-yl)pyrimidine-2,4(1H,3H)-dione

To a solution of 1-((2R,3R,4R,5R)-3-((tert-butyldimethylsilyl)oxy)-5-(((tert-butyldimethylsilyl)oxy)methyl)-4-hydroxytetrahydrofuran-2-yl)pyrimidine-2,4(1H,3H)-dione (1 equiv.) in DMSO at rt under Ar was added acetic acid (12 equiv.) followed by acetic anhydride (23 equiv.) and allowed to stir overnight. The reaction mixture was diluted with DCM and washed successively with saturated NaHCO₃ and brine. The combined organic phase was dried over Na₂SO₄, filtered, and purified by chromatography (0-5% MeOH in DCM) to afford 1-((2R,3R,4R,5R)-3-((tert-butyldimethylsilyl)oxy)-5-(((tert-butyldimethylsilyl)oxy)methyl)-4-((methylthio)methoxy)tetrahydrofuran-2-yl)pyrimidine-2,4(1H,3H)-dione.

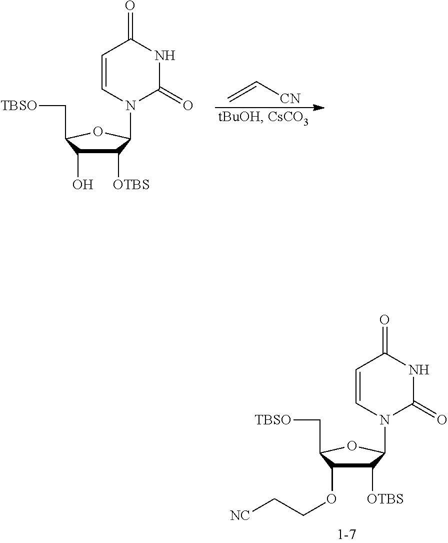

### 3-(((2R,3R,4R,5R)-4-((tert-butyldimethylsilyl)oxy)-2-(((tert-butyldimethylsilyl)oxy)methyl)-5-(2,4-dioxo-3,4-dihydropyrimidin-1(2H)-yl)tetrahydrofuran-3-yl)oxy)propanenitrile

To a solution of 1-((2R,3R,4R,5R)-3-((tert-butyldimethylsilyl)oxy)-5-(((tert-butyldimethylsilyl)oxy)methyl)-4-hydroxytetrahydrofuran-2-yl)pyrimidine-2,4(1H,3H)-dione (1 equiv.) in t-BuOH and acrylonitrile (20 eq) at rt under Ar was added cesium carbonate (1 equiv) and stirred for 2 h. The reaction mixture was diluted with DCM and MeOH (1:1, v/v) and filtered over silica. The precipitated solid was washed with additional DCM and MeOH (1:1, v/v, ×5). The filtrate was concentrated and purified by chromatography (0-5% MeOH in DCM) to afford 3-(((2R,3R,4R,5R)-4-((tert-butyldimethylsilyl)oxy)-2-(((tert-butyldimethylsilyl)oxy)methyl)-5-(2,4-dioxo-3,4-dihydropyrimidin-1(2H)-yl)tetrahydrofuran-3-yl)oxy)propanenitrile.

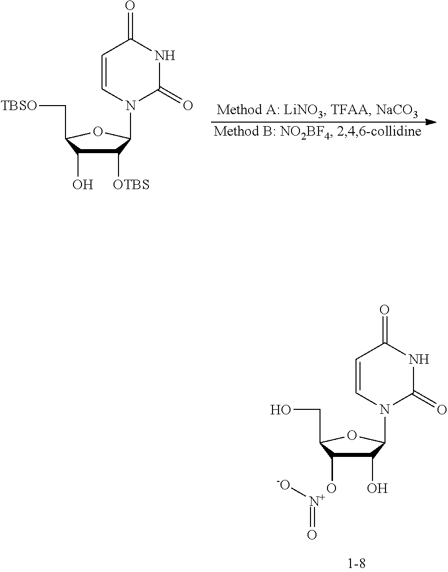

### (2R,3R,4R,5R)-4-((tert-butyldimethylsilyl)oxy)-2-(((tert-butyldimethylsilyl)oxy)methyl)-5-(2,4-dioxo-3,4-dihydropyrimidin-1(2H)-yl)tetrahydrofuran-3-yl nitrate

Method A: To a suspension of 1-((2R,3R,4R,5R)-3-((tert-butyldimethylsilyl)oxy)-5-(((tert-butyldimethylsilyl)oxy)methyl)-4-hydroxytetrahydrofuran-2-yl)pyrimidine-2,4(1H,3H)-dione (1 equiv.) and sodium carbonate (5 equiv.) in acetonitrile at 0° C. under Ar was added a solution containing lithium nitrate (2.1 equiv.) and trifluoroacetic anhydride (2 equiv.) in acetonitrile which was stirring for 20 min prior to the addition at 0° C. under Ar. The reaction mixture was allowed to gradually warm up to room temperate overnight. Most of acetonitrile was evaporated, the crude was re-suspended in EtOAc, and washed with water. The organic phase was dried over Na₂SO₄, filtered, and purified by chromatography (0-5% MeOH in DCM) to afford (2R,3R,4R,5R)-4-((tert-butyldimethylsilyl)oxy)-2-(((tert-butyldimethylsilyl)oxy)methyl)-5-(2,4-dioxo-3,4-dihydropyrimidin-1(2H)-yl)tetrahydrofuran-3-yl nitrate.

Method B: To a solution of 1-((2R,3R,4R,5R)-3-((tert-butyldimethylsilyl)oxy)-5-(((tert-butyldimethylsilyl)oxy)methyl)-4-hydroxytetrahydrofuran-2-yl)pyrimidine-2,4(1H,3H)-dione (1 equiv.), 2,4,6-collidine (6 equiv.) in dry acetonitrile was added an ice-cooled solution of nitronium tetrafluoroborate (3.5 equiv.) in dry acetonitrile via cannulation and allowed to stir for 1 h. Most of the acetonitrile was evaporated, and the crude was re-dissolved in EtOAc and sequentially washed with 5% citric acid (25 mL×3) and brine (25 mL×3). The organic phase was dried over Na₂SO₄, filtered, and purified by chromatography (0-5% MeOH in DCM) to afford (2R,3R,4R,5R)-4-((tert-butyldimethylsilyl)oxy)-2-(((tert-butyldimethylsilyl)oxy)methyl)-5-(2,4-dioxo-3,4-dihydropyrimidin-1(2H)-yl)tetrahydrofuran-3-yl nitrate.

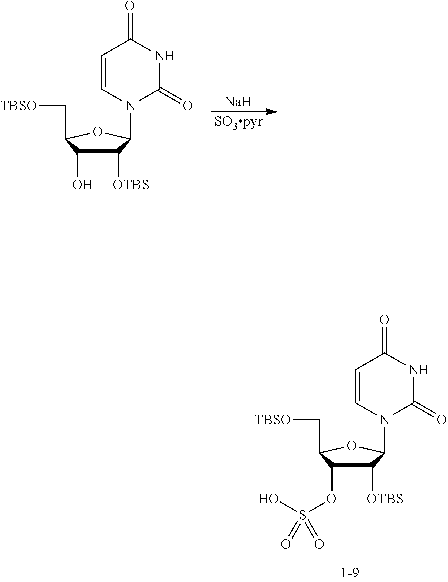

### (2R,3R,4R,5R)-4-((tert-butyldimethylsilyl)oxy)-2-(((tert-butyldimethylsilyl)oxy)methyl)-5-(2,4-dioxo-3,4-dihydropyrimidin-1(2H)-yl)tetrahydrofuran-3-yl hydrogen sulfate

To a solution of 1-((2R,3R,4R,5R)-3-((tert-butyldimethylsilyl)oxy)-5-(((tert-butyldimethylsilyl)oxy)methyl)-4-hydroxytetrahydrofuran-2-yl)pyrimidine-2,4(1H,3H)-dione (1 equiv.) in THE at 0° C. was added sodium hydride (60%, 1.2 equiv) and stirred for 20 min. To the resulting alkoxide was added pyridine sulfur trioxide (2.5 equv) and allowed to gradually warm up to rt overnight. The suspension was extracted between EtOAc and 5% citric acid. The aqueous phase was washed with EtOAc (×3) and the combined organic phase was dried over Na₂SO₄, filtered, and purified by chromatography (0-5% MeOH in DCM) to afford (2R,3R,4R,5R)-4-((tert-butyldimethylsilyl)oxy)-2-(((tert-butyldimethylsilyl)oxy)methyl)-5-(2,4-dioxo-3,4-dihydropyrimidin-1(2H)-yl)tetrahydrofuran-3-yl hydrogen sulfate.

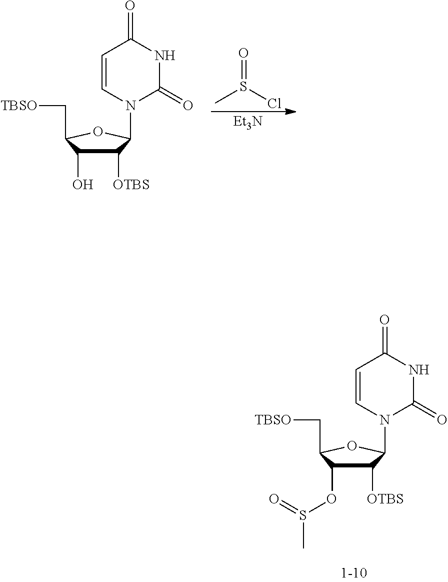

### (2R,3R,4R,5R)-4-((tert-butyldimethylsilyl)oxy)-2-(((tert-butyldimethylsilyl)oxy)methyl)-5-(2,4-dioxo-3,4-dihydropyrimidin-1(2H)-yl)tetrahydrofuran-3-yl methanesulfinate

To a solution of 1-((2R,3R,4R,5R)-3-((tert-butyldimethylsilyl)oxy)-5-(((tert-butyldimethylsilyl)oxy)methyl)-4-hydroxytetrahydrofuran-2-yl)pyrimidine-2,4(1H,3H)-dione (1 equiv.) and triethylamine (2.5 equiv.) in DCM at 0° C. was added methanesulfinic chloride (1.3 equiv.) and allowed to stir in a melting ice-bath for 5 h. The mixture was diluted with DCM and washed successively with water, saturated NaHCO₃, and brine. The organic phase was dried over Na₂SO₄, filtered, and purified by chromatography (0-5% MeOH in DCM) to afford (2R,3R,4R,5R)-4-((tert-butyldimethylsilyl)oxy)-2-(((tert-butyldimethylsilyl)oxy)methyl)-5-(2,4-dioxo-3,4-dihydropyrimidin-1(2H)-yl)tetrahydrofuran-3-yl methanesulfinate.

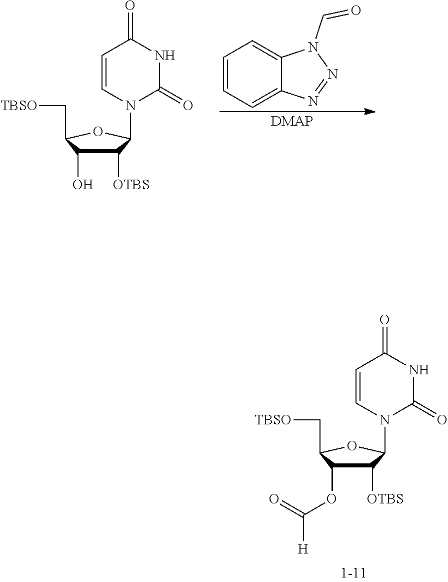

### (2R,3R,4R,5R)-4-((tert-butyldimethylsilyl)oxy)-2-(((tert-butyldimethylsilyl)oxy)methyl)-5-(2,4-dioxo-3,4-dihydropyrimidin-1(2H)-yl)tetrahydrofuran-3-yl formate

To a solution of 1-((2R,3R,4R,5R)-3-((tert-butyldimethylsilyl)oxy)-5-(((tert-butyldimethylsilyl)oxy)methyl)-4-hydroxytetrahydrofuran-2-yl)pyrimidine-2,4(1H,3H)-dione (1 equiv.) in THF at rt under Ar was added DMAP (0.08 equiv) followed by 1H-benzo[d][1,2,3]triazole-1-carbaldehyde (1 equiv.) and allowed to stir overnight. The contents were directly subjected to chromatography (0-5% MeOH in DCM) to afford (2R,3R,4R,5R)-4-((tert-butyldimethylsilyl)oxy)-2-(((tert-butyldimethylsilyl)oxy)methyl)-5-(2,4-dioxo-3,4-dihydropyrimidin-1(2H)-yl)tetrahydrofuran-3-yl formate.

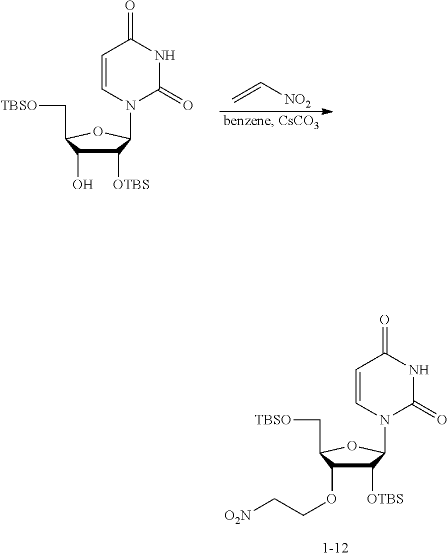

### 1-((2R,3R,4R,5R)-3-((tert-butyldimethylsilyl)oxy)-5-(((tert-butyldimethylsilyl)oxy)methyl)-4-(nitromethoxy)tetrahydrofuran-2-yl)pyrimidine-2,4(1H,3H)-dione

To a solution of 1-((2R,3R,4R,5R)-3-((tert-butyldimethylsilyl)oxy)-5-(((tert-butyldimethylsilyl)oxy)methyl)-4-hydroxytetrahydrofuran-2-yl)pyrimidine-2,4(1H,3H)-dione (1 equiv.) in benzene and nitroethylene (20 eq) at rt under Ar was added cesium carbonate (1 equiv) and stirred for 2 h. The reaction mixture was diluted with DCM and MeOH (1:1, v/v) and filtered over silica. The precipitated solid was washed with additional DCM and MeOH (1:1, v/v, ×5). The filtrate was concentrated and purified by chromatography (0-5% MeOH in DCM) to afford 1-((2R,3R,4R,5R)-3-((tert-butyldimethylsilyl)oxy)-5-(((tert-butyldimethylsilyl)oxy)methyl)-4-(nitromethoxy)tetrahydrofuran-2-yl)pyrimidine-2,4(1H,3H)-dione.

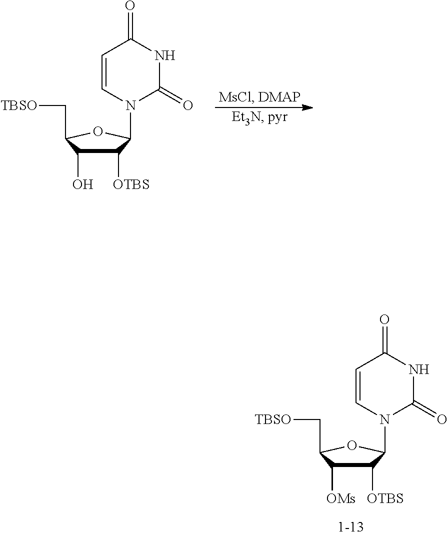

### (2R,3R,4R,5R)-4-((tert-butyldimethylsilyl)oxy)-2-(((tert-butyldimethylsilyl)oxy)methyl)-5-(2,4-dioxo-3,4-dihydropyrimidin-1(2H)-yl)tetrahydrofuran-3-yl methanesulfonate

To a solution 1-((2R,3R,4R,5R)-3-((tert-butyldimethylsilyl)oxy)-5-(((tert-butyldimethylsilyl)oxy)methyl)-4-hydroxytetrahydrofuran-2-yl)pyrimidine-2,4(1H,3H)-dione (1 equiv.) in DCM at rt under Ar was added triethylamine (4 equiv.), DMAP (2 equiv.), followed by MsCl (2 equiv.) and stirred for 2 h. The reaction mixture was diluted with DCM and washed sequentially with water and brine. The organic phase was dried over Na₂SO₄, filtered, and purified by chromatography (0-5% MeOH in DCM) to afford (2R,3R,4R,5R)-4-((tert-butyldimethylsilyl)oxy)-2-(((tert-butyldimethylsilyl)oxy)methyl)-5-(2,4-dioxo-3,4-dihydropyrimidin-1(2H)-yl)tetrahydrofuran-3-yl methanesulfonate.

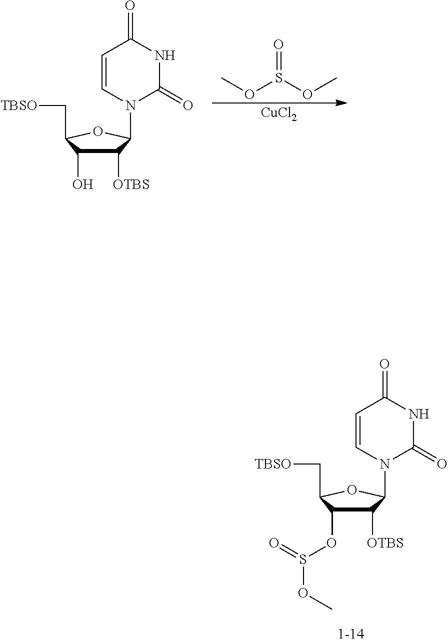

### (2R,3R,4R,5R)-4-((tert-butyldimethylsilyl)oxy)-2-(((tert-butyldimethylsilyl)oxy)methyl)-5-(2,4-dioxo-3,4-dihydropyrimidin-1(2H)-yl)tetrahydrofuran-3-yl methyl sulfite

To a suspension of copper (II) chloride (0.25 equiv.) in 1,4-dioxane at rt under Ar was added 1-((2R,3R,4R,5R)-3-((tert-butyldimethylsilyl)oxy)-5-(((tert-butyldimethylsilyl)oxy)methyl)-4-hydroxytetrahydrofuran-2-yl)pyrimidine-2,4(1H,3H)-dione (1 equiv.), and dimethyl sulfite (5 equiv.). The reaction mixture was stirred vigorously at 70° C., then increased gradually to 90° C. for 45 hours. After removal of dioxane, the crude was suspended in DCM and the precipitated copper was removed by filtration. The filtrate was concentrated and directly subjected to chromatography (0-5% MeOH in DCM) to afford (2R,3R,4R,5R)-4-((tert-butyldimethylsilyl)oxy)-2-(((tert-butyldimethylsilyl)oxy)methyl)-5-(2,4-dioxo-3,4-dihydropyrimidin-1(2H)-yl)tetrahydrofuran-3-yl methyl sulfite.

[Reference: Tet. 2015, 71, 8905-8910]

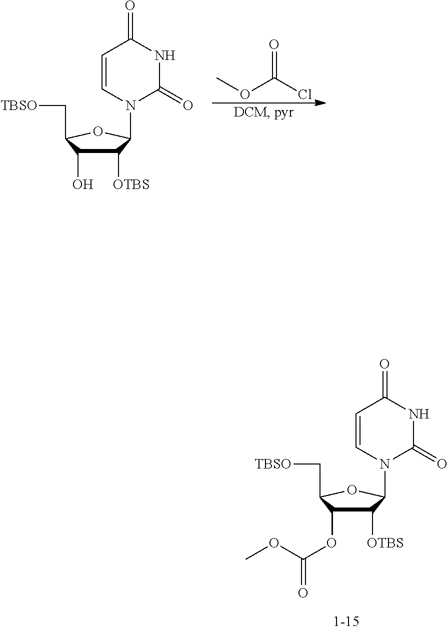

### (2R,3R,4R,5R)-4-((tert-butyldimethylsilyl)oxy)-2-(((tert-butyldimethylsilyl)oxy)methyl)-5-(2,4-dioxo-3,4-dihydropyrimidin-1(2H)-yl)tetrahydrofuran-3-yl methyl carbonate

To a solution of 1-((2R,3R,4R,5R)-3-((tert-butyldimethylsilyl)oxy)-5-(((tert-butyldimethylsilyl)oxy)methyl)-4-hydroxytetrahydrofuran-2-yl)pyrimidine-2,4(1H,3H)-dione (1 equiv.) in DCM at rt under Ar was added pyridine (1.5 equiv.) followed by methyl chloroformate (1 equiv.) and stirred for 8 h. The reaction mixture was diluted with DCM and washed with water. The organic phase was dried over Na₂SO₄, filtered, and purified by chromatography (0-5% MeOH in DCM) to afford (2R,3R,4R,5R)-4-((tert-butyldimethylsilyl)oxy)-2-(((tert-butyldimethylsilyl)oxy)methyl)-5-(2,4-dioxo-3,4-dihydropyrimidin-1(2H)-yl)tetrahydrofuran-3-yl methyl carbonate.

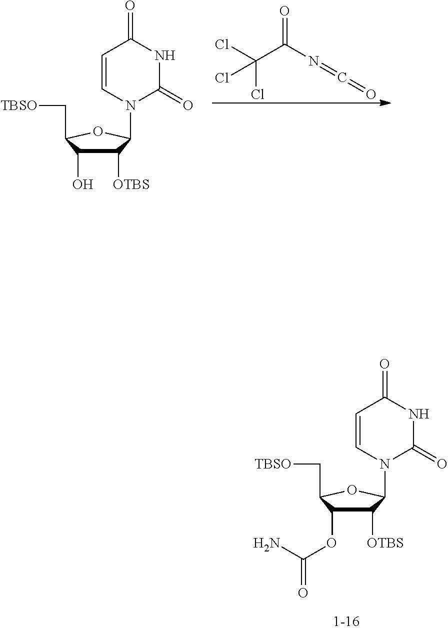

### (2R,3R,4R,5R)-4-((tert-butyldimethylsilyl)oxy)-2-(((tert-butyldimethylsilyl)oxy)methyl)-5-(2,4-dioxo-3,4-dihydropyrimidin-1(2H)-yl)tetrahydrofuran-3-yl carbamate

To a solution of 1-((2R,3R,4R,5R)-3-((tert-butyldimethylsilyl)oxy)-5-(((tert-butyldimethylsilyl)oxy)methyl)-4-hydroxytetrahydrofuran-2-yl)pyrimidine-2,4(1H,3H)-dione (1 equiv.) in DCM at 0° C. under Ar was added trichloroisocyanate (1.5 equiv.) and stirred for 1 h. To the reaction mixture was added excess methanolic ammonia (7N) and stirred for 15 min. The contents were concentrated and subjected to chromatography (0-5% MeOH in DCM) to afford 20 (2R,3R,4R,5R)-4-((tert-butyldimethylsilyl)oxy)-2-(((tert-butyldimethylsilyl)oxy)methyl)-5-(2,4-dioxo-3,4-dihydropyrimidin-1(2H)-yl)tetrahydrofuran-3-yl carbamate.

[Reference: Organic Letters (2008), 10(11), 2179-2182]

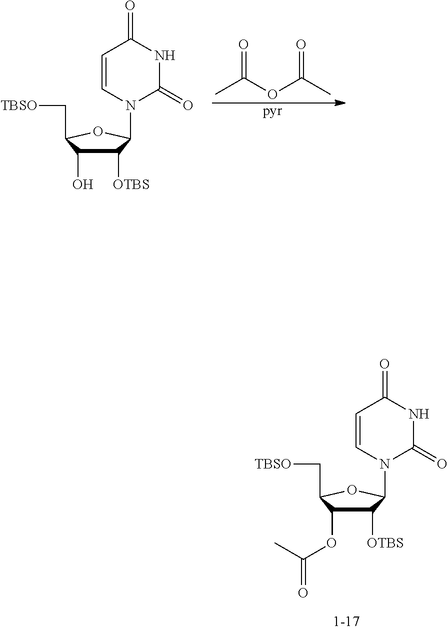

### (2R,3R,4R,5R)-4-((tert-butyldimethylsilyl)oxy)-2-(((tert-butyldimethylsilyl)oxy)methyl)-5-(2,4-dioxo-3,4-dihydropyrimidin-1(2H)-yl)tetrahydrofuran-3-yl acetate

To a solution of 1-((2R,3R,4R,5R)-3-((tert-butyldimethylsilyl)oxy)-5-(((tert-butyldimethylsilyl)oxy)methyl)-4-hydroxytetrahydrofuran-2-yl)pyrimidine-2,4(1H,3H)-dione (1 equiv.) in anhydrous pyridine at rt under Ar was added acetic anhydride (1.2 equiv.) and stirred for 30 h. A further aliquot of acetic anhydride (1.2 equiv.) was added and allowed to stir for another 72 h. The reaction mixture was treated with water (10 equiv.) and then evaporated. The crude residue was taken up in DCM and the solution washed with 1 M hydrochloric acid (×3), then dried over 10 anhydrous Na₂SO₄, filtered and evaporated to give (2R,3R,4R,5R)-4-((tert-butyldimethylsilyl)oxy)-2-(((tert-butyldimethylsilyl)oxy)methyl)-5-(2,4-dioxo-3,4-dihydropyrimidin-1(2H)-yl)tetrahydrofuran-3-yl acetate without further purification.

[Reference: WO2002018404]

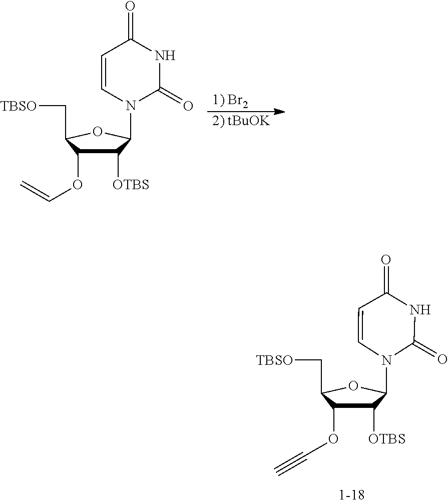

### 1-((2R,3R,4R,5R)-3-((tert-butyldimethylsilyl)oxy)-5-(((tert-butyldimethylsilyl)oxy)methyl)-4-(ethynyloxy)tetrahydrofuran-2-yl)pyrimidine-2,4(1H,3H)-dione

To a solution of 1-((2R,3R,4R,5R)-4-(allyloxy)-3-((tert-butyldimethylsilyl)oxy)-5-(((tert-butyldimethylsilyl)oxy)methyl)tetrahydrofuran-2-yl)pyrimidine-2,4(1H,3H)-dione (1 equiv.) in DCM at −78° C. under Ar was added bromine (10 equiv.) until persistent bromine and the reaction mixture was then decolorized with ethyl vinyl ether. Solid tBuOK (5 equiv.) was added to the reaction mixture at the same temperature and stirred for 1 h. The reaction was quenched with ice-cold water. The organic later was separated and the aqueous layer was extracted with DCM. The combined organic phase was dried over Na₂SO₄, filtered, and purified by chromatography (0-5% MeOH in DCM) to afford 1-((2R,3R,4R,5R)-3-((tert-butyldimethylsilyl)oxy)-5-(((tert-butyldimethylsilyl)oxy)methyl)-4-(ethynyloxy)tetrahydrofuran-2-yl)pyrimidine-2,4(1H,3H)-dione.

[Reference: Tetrahedron Vol. 43, No. 10, pp. 2311-2316, 1987]

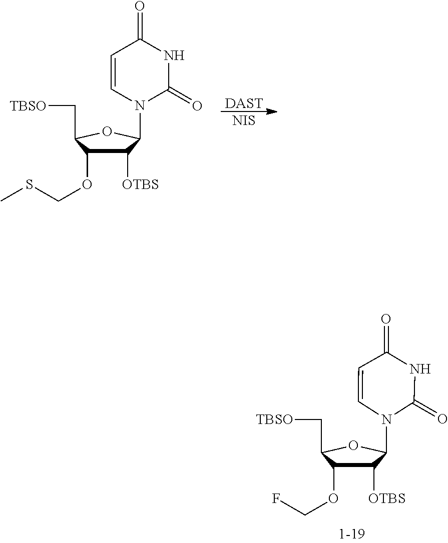

### 1-((2R,3R,4R,5R)-3-((tert-butyldimethylsilyl)oxy)-5-(((tert-butyldimethylsilyl)oxy)methyl)-4-(fluoromethoxy)tetrahydrofuran-2-yl)pyrimidine-2,4(1H,3H)-dione

To a solution of 1-((2R,3R,4R,5R)-3-((tert-butyldimethylsilyl)oxy)-5-(((tert-butyldimethylsilyl)oxy)methyl)-4-((methylthio)methoxy)tetrahydrofuran-2-yl)pyrimidine-2,4(1H,3H)-dione (1 equiv.) in DCM at 0° C. under Ar was added N-Iodosuccinimide (1.5 equiv.) and stirred for 10 min. To the reaction mixture was then added DAST (1.5 equiv.) and allowed to stir for 1 h. The reaction mixture was diluted with DCM and washed sequentially with saturated sodium bicarbonate, 10% sodium sulfite, and brine. The organic phase was dried over Na₂SO₄, filtered, and purified by chromatography (0-5% MeOH in DCM) to afford 1-((2R,3R,4R,5R)-3-((tert-butyldimethylsilyl)oxy)-5-(((tert-butyldimethylsilyl)oxy)methyl)-4-(fluoromethoxy)tetrahydrofuran-2-yl)pyrimidine-2,4(1H,3H)-dione.

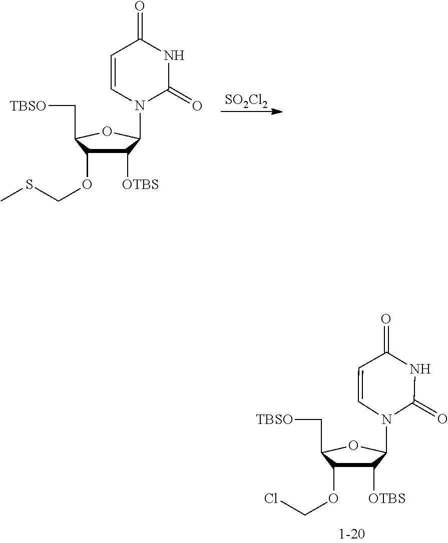

### 1-((2R,3R,4R,5R)-3-((tert-butyldimethylsilyl)oxy)-5-(((tert-butyldimethylsilyl)oxy)methyl)-4-(chloromethoxy)tetrahydrofuran-2-yl)pyrimidine-2,4(1H,3H)-dione

To a solution of 1-((2R,3R,4R,5R)-3-((tert-butyldimethylsilyl)oxy)-5-(((tert-butyldimethylsilyl)oxy)methyl)-4-((methylthio)methoxy)tetrahydrofuran-2-yl)pyrimidine-2,4(1H,3H)-dione (1 equiv.) in DCM at 0° C. under Ar was added sulfuryl chloride (1.1 equiv.) and allowed to stir for 1 h. The reaction mixture was concentrated and dried over vacuum to afford intermediate compound 1-((2R,3R,4R,5R)-3-((tert-butyldimethylsilyl)oxy)-5-(((tert-butyldimethylsilyl)oxy)methyl)-4-(chloromethoxy)tetrahydrofuran-2-yl)pyrimidine-2,4(1H,3H)-dione (compound 1-20). No further purification was performed.

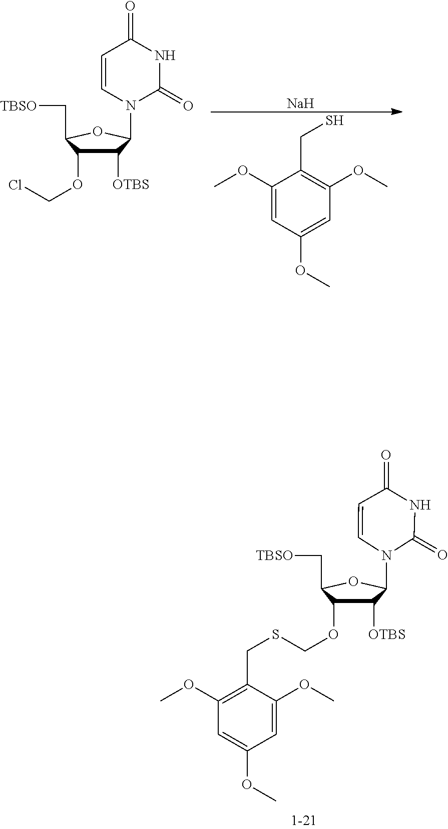

### 1-((2R,3R,4R,5R)-3-((tert-butyldimethylsilyl)oxy)-5-(((tert-butyldimethylsilyl)oxy)methyl)-4-(((2,4,6-trimethoxybenzyl)thio)methoxy)tetrahydrofuran-2-yl)pyrimidine-2,4(1H,3H)-dione

To a solution of (2,4,6-trimethoxyphenyl)methanethiol (2.5 equiv.) in DMF at 0° C. under Ar was added sodium hydride (60%, 2.4 equiv.) and stirred for 30 min. The previously dried crude (1-20) was dissolved in DMF and added to the reaction mixture which was allowed to stir for 3 h while gradually allowing to warm to rt. The contents were diluted with EtOAc and washed with brine (×5). The organic phase was dried over Na₂SO₄, filtered, and purified by chromatography (0-5% MeOH in DCM) to afford intermediate 1-((2R,3R,4R,5R)-3-((tert-butyldimethylsilyl)oxy)-5-(((tert-butyldimethylsilyl)oxy)methyl)-4-(((2,4,6-trimethoxybenzyl)thio)methoxy)tetrahydrofuran-2-yl)pyrimidine-2,4(1H,3H)-dione (compound 1-21).

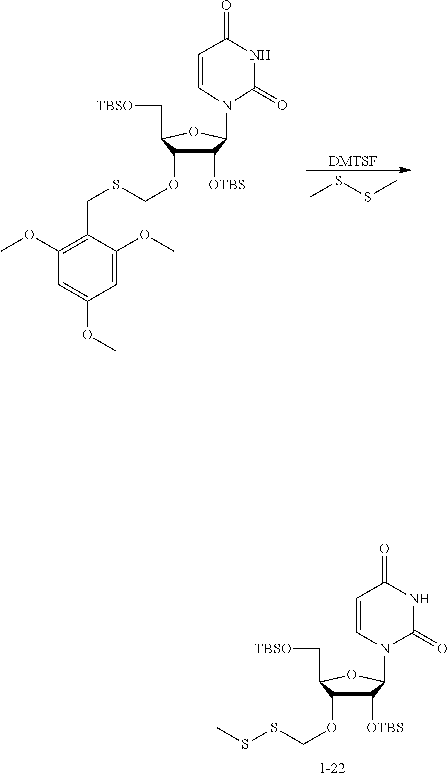

### 1-((2R,3R,4R,5R)-3-((tert-butyldimethylsilyl)oxy)-5-(((tert-butyldimethylsilyl)oxy)methyl)-4-((methyldisulfaneyl)methoxy)tetrahydrofuran-2-yl)pyrimidine-2,4(1H,3H)-dione

To a solution of 1-((2R,3R,4R,5R)-3-((tert-butyldimethylsilyl)oxy)-5-(((tert-butyldimethylsilyl)oxy)methyl)-4-(((2,4,6-trimethoxybenzyl)thio)methoxy)tetrahydrofuran-2-yl)pyrimidine-2,4(1H,3H)-dione (compound 1-21) in THE at rt under Ar was added 1,2-dimethylsulfane (26 equiv.) followed by dimethyl(methylthio)sulfonium tetrafluoroborate (2.5 equiv.) and allowed to stir for 1 h. The reaction mixture was quenched with saturated sodium bicarbonate and extracted twice with EtOAc. The combined organic phase was dried over Na₂SO₄, filtered, and purified by chromatography (0-5% MeOH in DCM) to afford the final product 1-((2R,3R,4R,5R)-3-((tert-butyldimethylsilyl)oxy)-5-(((tert-butyldimethylsilyl)oxy)methyl)-4-((methyldisulfaneyl)methoxy)tetrahydrofuran-2-yl)pyrimidine-2,4(1H,3H)-dione.

[Reference: KA647 & KA651]

### Example 2: The Uridine Analogs (1-1 Through 1-19 and 1-22 Above) were Deblocked Using the Following Methods to Obtain Analogs 1-23 Through 1-41 Above

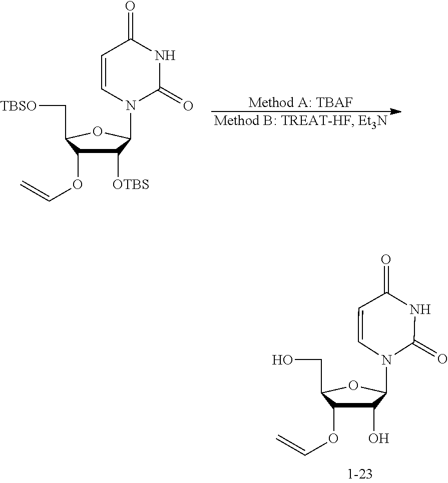

### 1-((2R,3R,4S,5R)-3-hydroxy-5-(hydroxymethyl)-4-(vinyloxy)tetrahydrofuran-2-yl)pyrimidine-2,4(1H,3H)-dione

Method A: To a solution of 1-((2R,3R,4R,5R)-3-((tert-butyldimethylsilyl)oxy)-5-(((tert-butyldimethylsilyl)oxy)methyl)-4-(vinyloxy)tetrahydrofuran-2-yl)pyrimidine-2,4(1H,3H)-dione (1 equiv.) in THE at rt under Ar was added TBAF (1.0 M, 2.5 equiv.) and stirred for 1 h. The crude was concentrated to an oil and directly subjected to chromatography (0-10% MeOH in DCM) to afford 1-((2R,3R,4S,5R)-3-hydroxy-5-(hydroxymethyl)-4-(vinyloxy)tetrahydrofuran-2-yl)pyrimidine-2,4(1H,3H)-dione.

Method B: To a solution of 1-((2R,3R,4R,5R)-3-((tert-butyldimethylsilyl)oxy)-5-(((tert-butyldimethylsilyl)oxy)methyl)-4-(vinyloxy)tetrahydrofuran-2-yl)pyrimidine-2,4(1H,3H)-dione (1 equiv.) in THE at rt under Ar were added triethylamine (4 equiv.) followed by triethylamine trihydrogenfluoride (2 equiv.) and allowed to stir overnight. The crude was concentrated to an oil and directly subjected to chromatography (0-10% MeOH in DCM) to afford 1-((2R,3R,4S,5R)-3-hydroxy-5-(hydroxymethyl)-4-(vinyloxy)tetrahydrofuran-2-yl)pyrimidine-2,4(1H,3H)-dione.

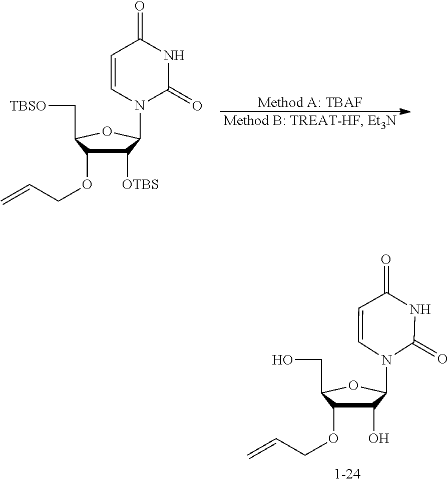

### 1-((2R,3R,4S,5R)-4-(allyloxy)-3-hydroxy-5-(hydroxymethyl)tetrahydrofuran-2-yl)pyrimidine-2,4(1H,3H)-dione

Same procedure as for compound 1-23.

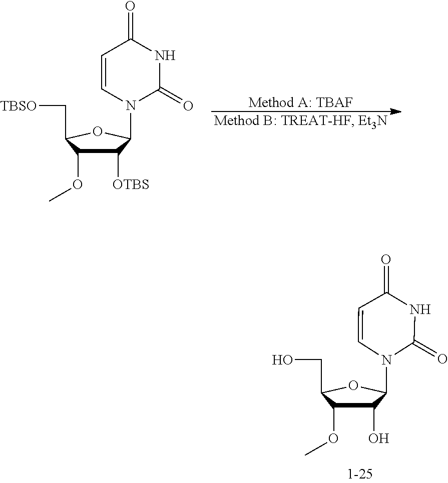

### 1-((2R,3R,4S,5R)-3-hydroxy-5-(hydroxymethyl)-4-methoxytetrahydrofuran-2-yl)pyrimidine-2,4(1H,3H)-dione

Same procedure as for compound 1-23.

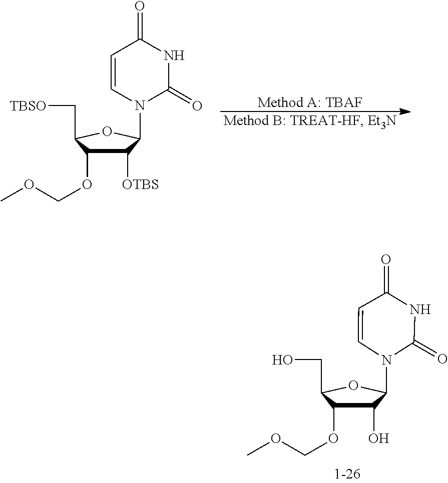

### 1-((2R,3R,4S,5R)-3-hydroxy-5-(hydroxymethyl)-4-(methoxymethoxy)tetrahydrofuran-2-yl)pyrimidine-2,4(1H,3H)-dione

Same procedure as for compound 1-23.

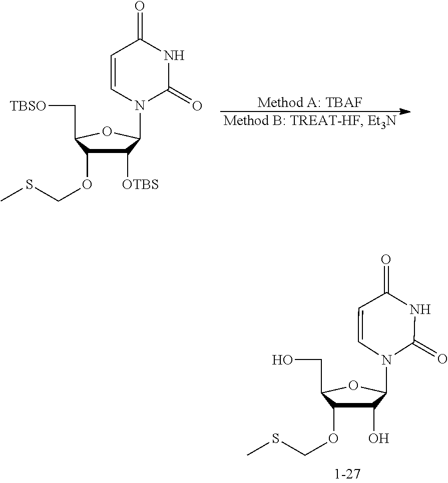

### 1-((2R,3R,4S,5R)-3-hydroxy-5-(hydroxymethyl)-4-((methylthio)methoxy)tetrahydrofuran-2-yl)pyrimidine-2,4(1H,3H)-dione

Same procedure as for compound 1-23.

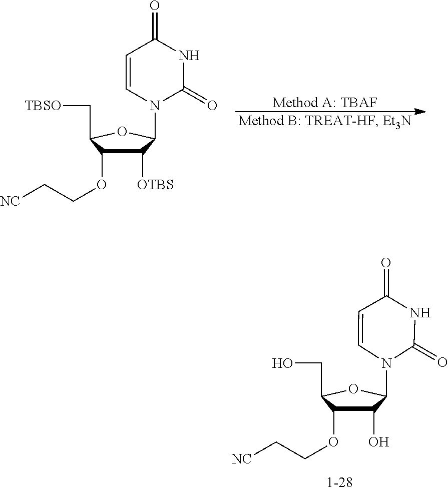

### 3-(((2R,3S,4R,5R)-5-(2,4-dioxo-3,4-dihydropyrimidin-1(2H)-yl)-4-hydroxy-2-(hydroxymethyl)tetrahydrofuran-3-yl)oxy)propanenitrile

Same procedure as for compound 1-23.

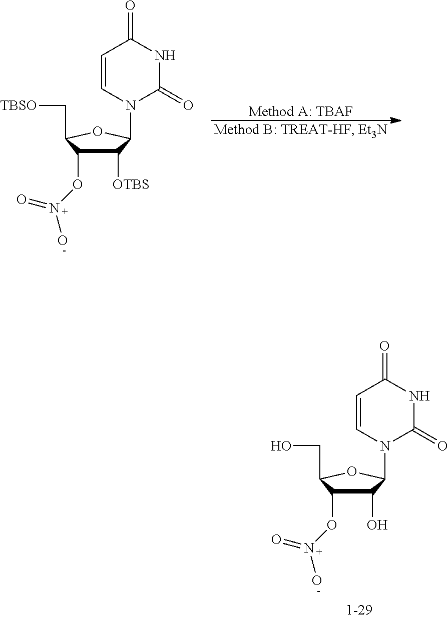

### (2R,3S,4R,5R)-5-(2,4-dioxo-3,4-dihydropyrimidin-1(2H)-yl)-4-hydroxy-2-(hydroxymethyl)tetrahydrofuran-3-yl nitrate

Same procedure as for compound 1-23.

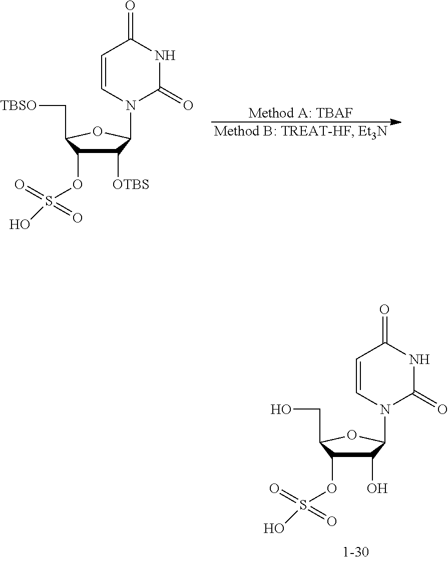

### (2R,3 S,4R,5R)-5-(2,4-dioxo-3,4-dihydropyrimidin-1(2H)-yl)-4-hydroxy-2-(hydroxymethyl)tetrahydrofuran-3-yl hydrogen sulfate

Same procedure as for compound 1-23.

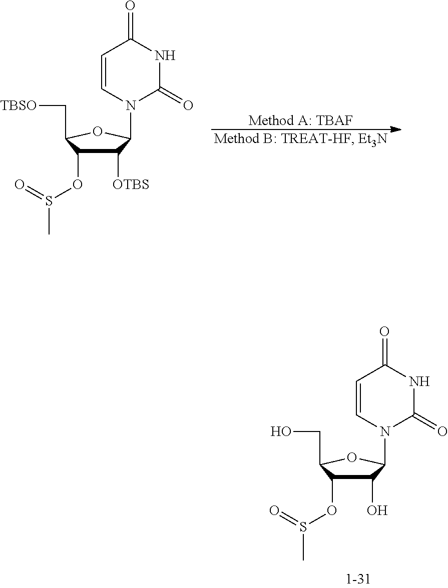

### (2R,3S,4R,5R)-5-(2,4-dioxo-3,4-dihydropyrimidin-1(2H)-yl)-4-hydroxy-2-(hydroxymethyl)tetrahydrofuran-3-yl methanesulfinate

Same procedure as for compound 1-23.

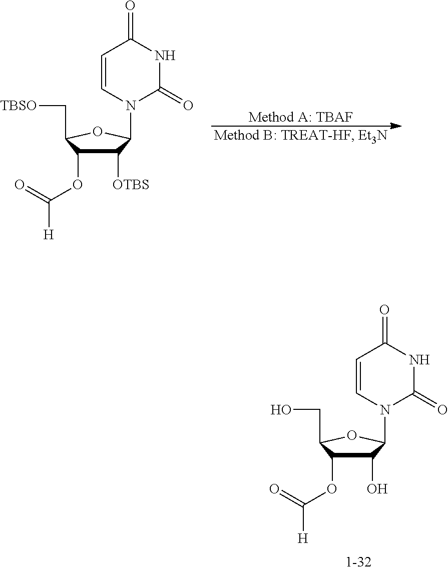

### (2R,3S,4R,5R)-5-(2,4-dioxo-3,4-dihydropyrimidin-1(2H)-yl)-4-hydroxy-2-(hydroxymethyl)tetrahydrofuran-3-yl formate

Same procedure as for compound 1-23.

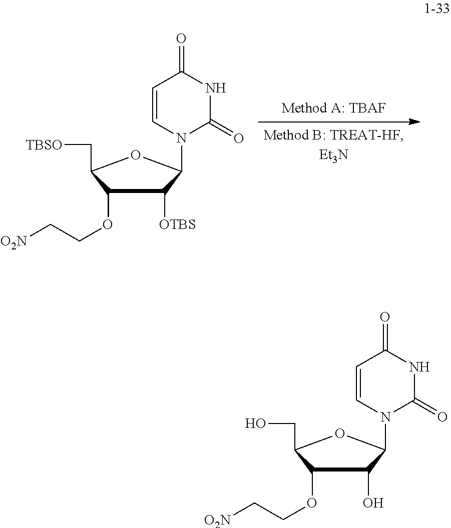

### 1-((2R,3R,4S,5R)-3-hydroxy-5-(hydroxymethyl)-4-(2-nitroethoxy)tetrahydrofuran-2-yl)pyrimidine-2,4(1H,3H)-dione

Same procedure as for compound 1-23.

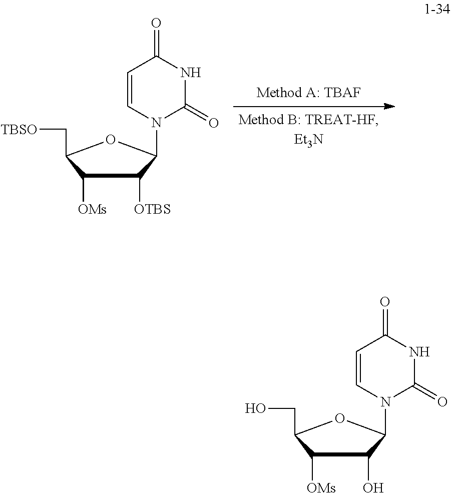

### (2R,3S,4R,5R)-5-(2,4-dioxo-3,4-dihydropyrimidin-1(2H)-yl)-4-hydroxy-2-(hydroxymethyl)tetrahydrofuran-3-yl methanesulfonate

Same procedure as for compound 1-23.

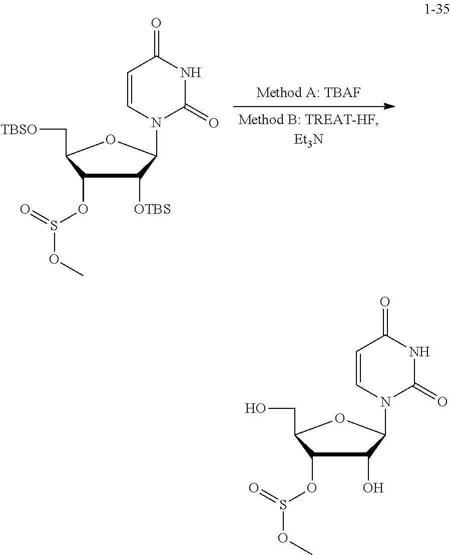

### (2R,3S,4R,5R)-5-(2,4-dioxo-3,4-dihydropyrimidin-1(2H)-yl)-4-hydroxy-2-(hydroxymethyl)tetrahydrofuran-3-yl methyl sulfite

Same procedure as for compound 1-23.

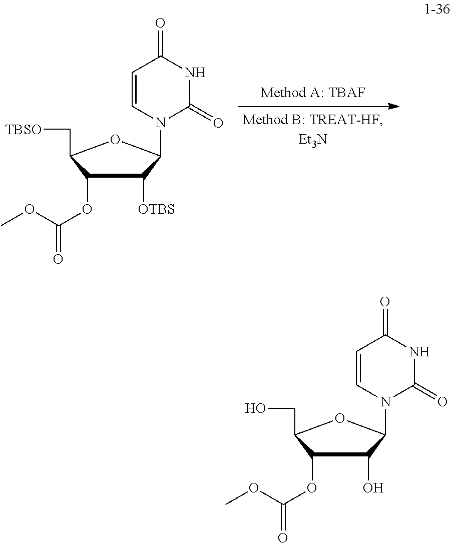

### (2R,3S,4R,5R)-5-(2,4-dioxo-3,4-dihydropyrimidin-1(2H)-yl)-4-hydroxy-2-(hydroxymethyl)tetrahydrofuran-3-yl methyl carbonate

Same procedure as for compound 1-23.

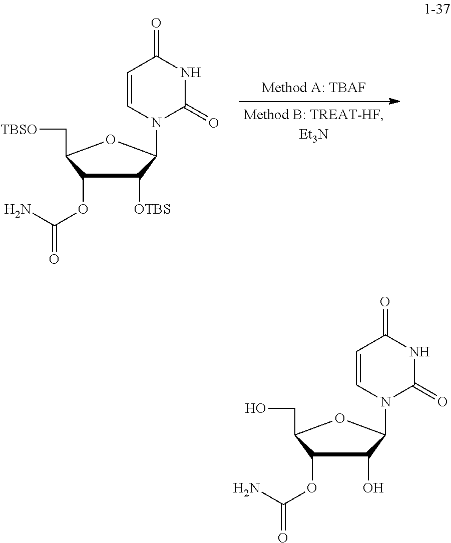

### (2R,3S,4R,5R)-5-(2,4-dioxo-3,4-dihydropyrimidin-1(2H)-yl)-4-hydroxy-2-(hydroxymethyl)tetrahydrofuran-3-yl carbamate

Same procedure as for compound 1-23.

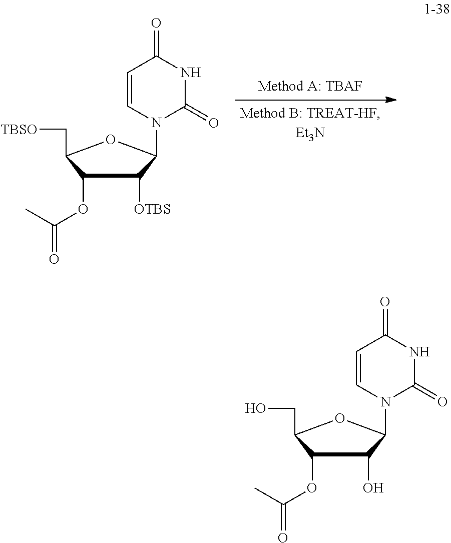

### (2R,3 S,4R,5R)-5-(2,4-dioxo-3,4-dihydropyrimidin-1(2H)-yl)-4-hydroxy-2-(hydroxymethyl)tetrahydrofuran-3-yl acetate

Same procedure as for compound 1-23.

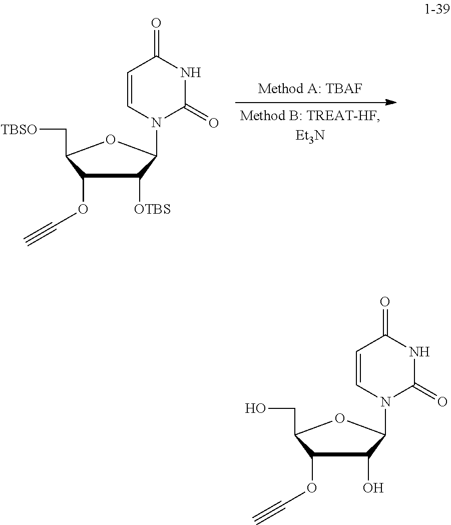

### 1-((2R,3R,4S,5R)-4-(ethynyloxy)-3-hydroxy-5-(hydroxymethyl)tetrahydrofuran-2-yl)pyrimidine-2,4(1H,3H)-dione

Same procedure as for compound 1-23.

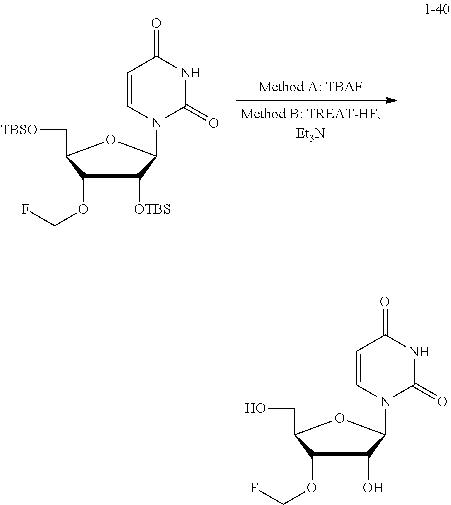

### 1-((2R,3R,4S,5R)-4-(fluoromethoxy)-3-hydroxy-5-(hydroxymethyl)tetrahydrofuran-2-yl)pyrimidine-2,4(1H,3H)-dione

Same procedure as for compound 1-23.

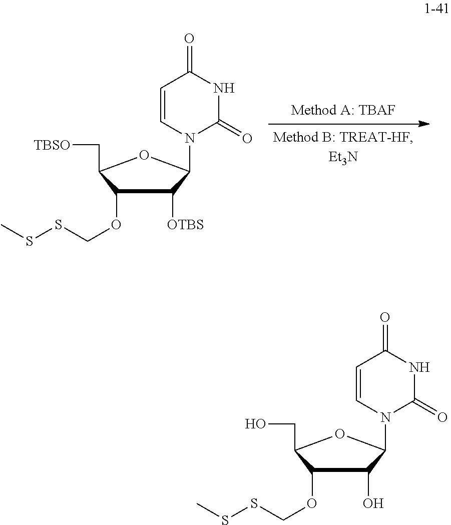

### 1-((2R,3R,4S,5R)-3-hydroxy-5-(hydroxymethyl)-4-((methyldisulfaneyl)methoxy)tetrahydrofuran-2-yl)pyrimidine-2,4(1H,3H)-dione

Same procedure as for compound 1-23.

### Example 3: A Common Cytidine Intermediate was Synthesized as Follows

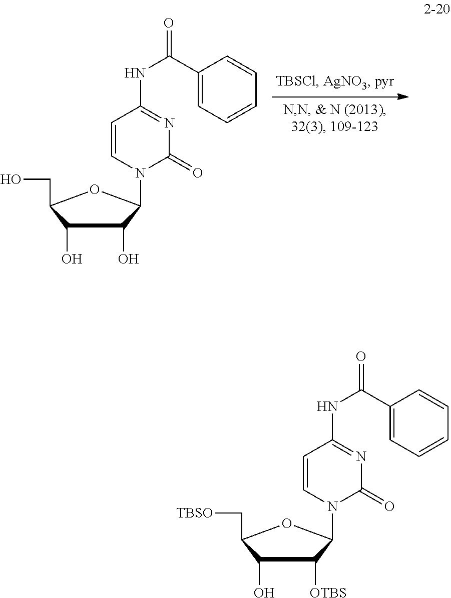

### N-(1-((2R,3R,4R,5R)-3-((tert-butyldimethylsilyl)oxy)-4-hydroxy-5-(hydroxymethyl)tetrahydrofuran-2-yl)-2-oxo-1,2-dihydropyrimidin-4-yl)benzamide

Same procedure as compound 1-20

### Example 4: Analogs (2-1 Through 2-19 Above) were Prepared Using the Following Methods

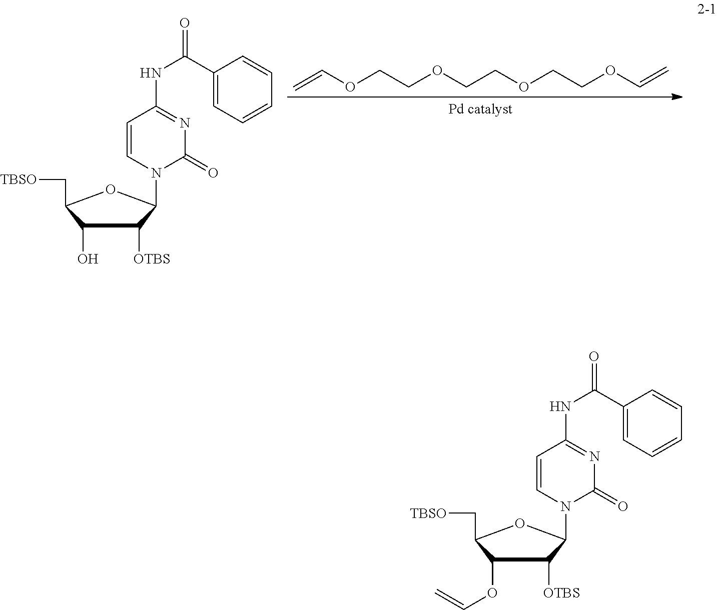

### N-(1-((2R,3R,4R,5R)-3-((tert-butyldimethylsilyl)oxy)-5-(((tert-butyldimethylsilyl)oxy)methyl)-4-(vinyloxy)tetrahydrofuran-2-yl)-2-oxo-1,2-dihydropyrimidin-4-yl)benzamide

Same procedure as compound 1-1.

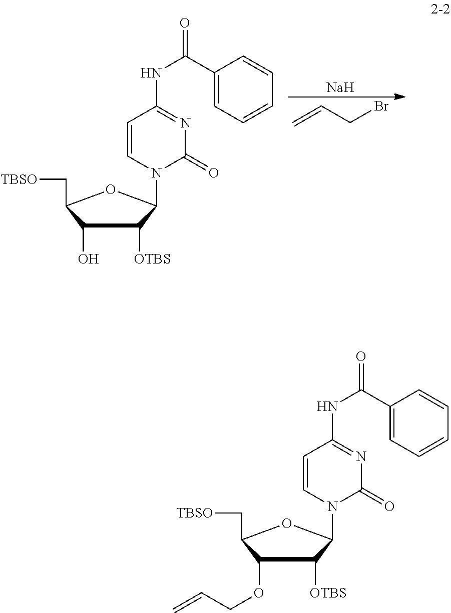

### N-(1-((2R,3R,4R,5R)-4-(allyloxy)-3-((tert-butyldimethylsilyl)oxy)-5-(((tert-butyldimethylsilyl)oxy)methyl)tetrahydrofuran-2-yl)-2-oxo-1,2-dihydropyrimidin-4-yl)benzamide

Same procedure as compound 1-2.

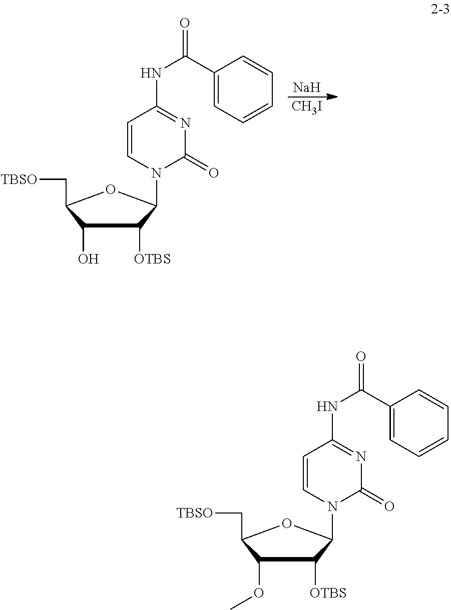

### N-(1-((2R,3R,4R,5R)-3-((tert-butyldimethylsilyl)oxy)-5-(((tert-butyldimethylsilyl)oxy)methyl)-4-methoxytetrahydrofuran-2-yl)-2-oxo-1,2-dihydropyrimidin-4-yl)benzamide

Same procedure as compound 1-3.

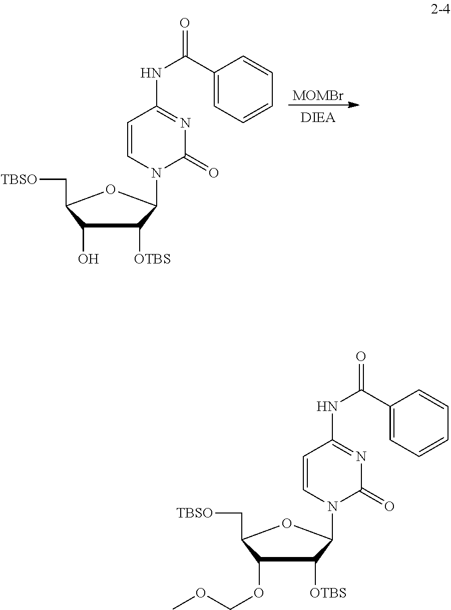

### N-(1-((2R,3R,4R,5R)-3-((tert-butyldimethylsilyl)oxy)-5-(((tert-butyldimethylsilyl)oxy)methyl)-4-(methoxymethoxy)tetrahydrofuran-2-yl)-2-oxo-1,2-dihydropyrimidin-4-yl)benzamide

Same procedure as compound 1-4.

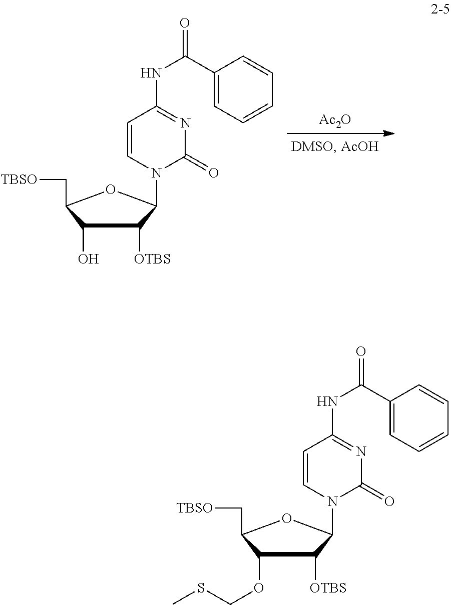

### N-(1-((2R,3R,4R,5R)-3-((tert-butyldimethylsilyl)oxy)-5-(((tert-butyldimethylsilyl)oxy)methyl)-4-((methylthio)methoxy)tetrahydrofuran-2-yl)-2-oxo-1,2-dihydropyrimidin-4-yl)benzamide

Same procedure as compound 1-5.

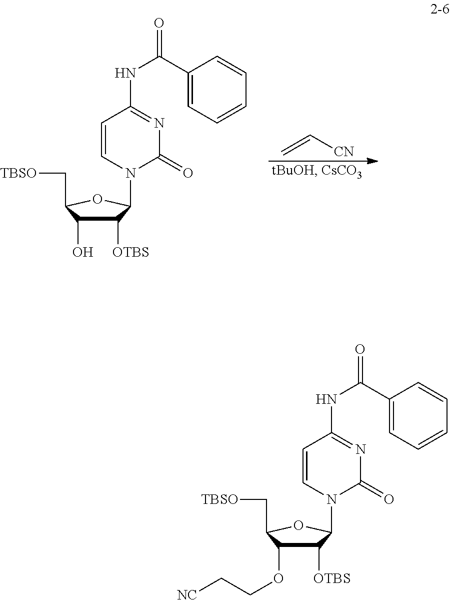

### N-(1-((2R,3R,4R,5R)-3-((tert-butyldimethylsilyl)oxy)-5-(((tert-butyldimethylsilyl)oxy)methyl)-4-(2-cyanoethoxy)tetrahydrofuran-2-yl)-2-oxo-1,2-dihydropyrimidin-4-yl)benzamide

Same procedure as compound 1-6.

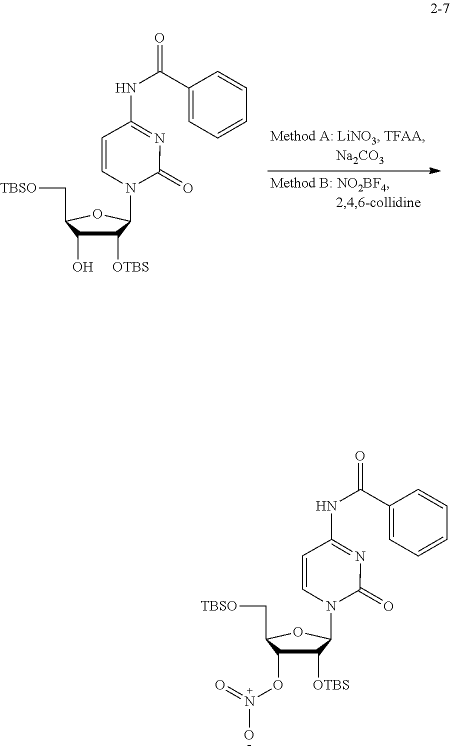

### (2R,3R,4R,5R)-5-(4-benzamido-2-oxopyrimidin-1(2H)-yl)-4-((tert-butyldimethylsilyl)oxy)-2-(((tert-butyldimethylsilyl)oxy)methyl)tetrahydrofuran-3-yl nitrate

Same procedure as compound 1-7.

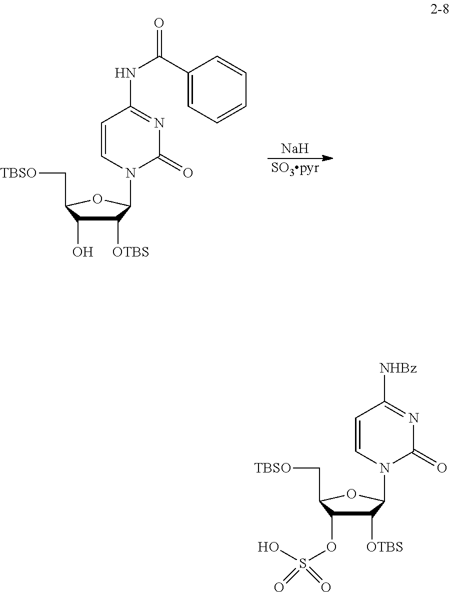

### (2R,3R,4R,5R)-5-(4-benzamido-2-oxopyrimidin-1(2H)-yl)-4-((tert-butyldimethylsilyl)oxy)-2-(((tert-butyldimethylsilyl)oxy)methyl)tetrahydrofuran-3-yl hydrogen sulfate

Same procedure as compound 1-8.

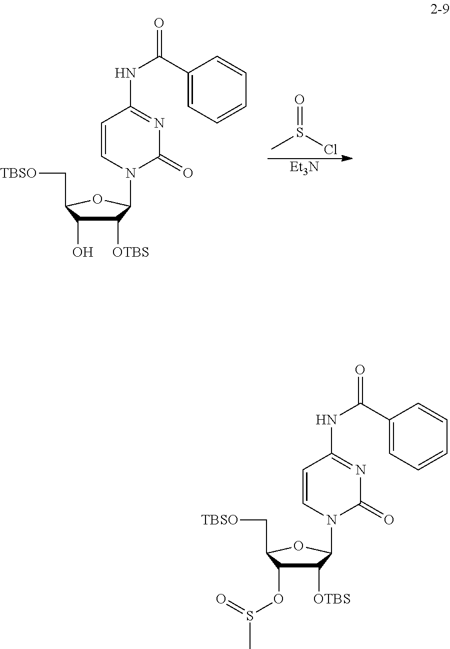

### (2R,3R,4R,5R)-5-(4-benzamido-2-oxopyrimidin-1(2H)-yl)-4-((tert-butyldimethylsilyl)oxy)-2-(((tert-butyldimethylsilyl)oxy)methyl)tetrahydrofuran-3-yl methanesulfinate

Same procedure as compound 1-9.

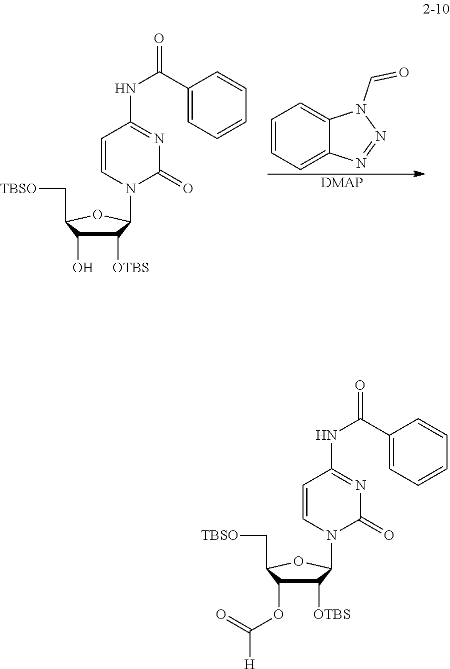

### (2R,3R,4R,5R)-5-(4-benzamido-2-oxopyrimidin-1(2H)-yl)-4-((tert-butyldimethylsilyl)oxy)-2-(((tert-butyldimethylsilyl)oxy)methyl)tetrahydrofuran-3-yl formate

Same procedure as compound 1-10.

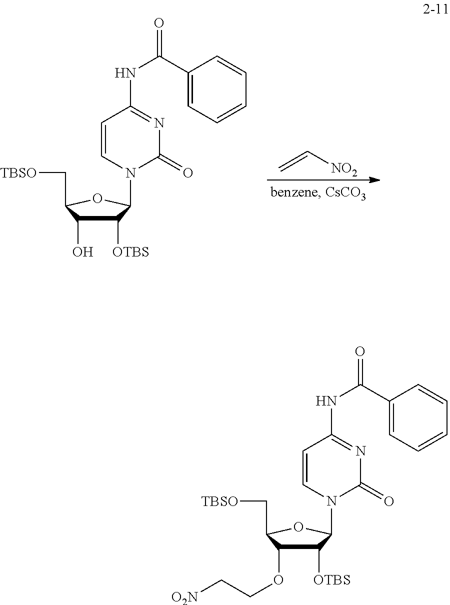

### N-(1-((2R,3R,4R,5R)-3-((tert-butyldimethylsilyl)oxy)-5-(((tert-butyldimethylsilyl)oxy)methyl)-4-(2-nitroethoxy)tetrahydrofuran-2-yl)-2-oxo-1,2-dihydropyrimidin-4-yl)benzamide

Same procedure as compound 1-11.

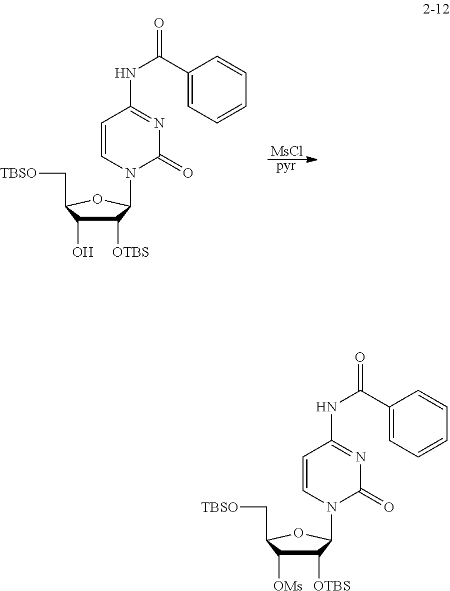

### (2R,3R,4R,5R)-5-(4-benzamido-2-oxopyrimidin-1(2H)-yl)-4-((tert-butyldimethylsilyl)oxy)-2-(((tert-butyldimethylsilyl)oxy)methyl)tetrahydrofuran-3-yl methanesulfonate

Same procedure as compound 1-12.

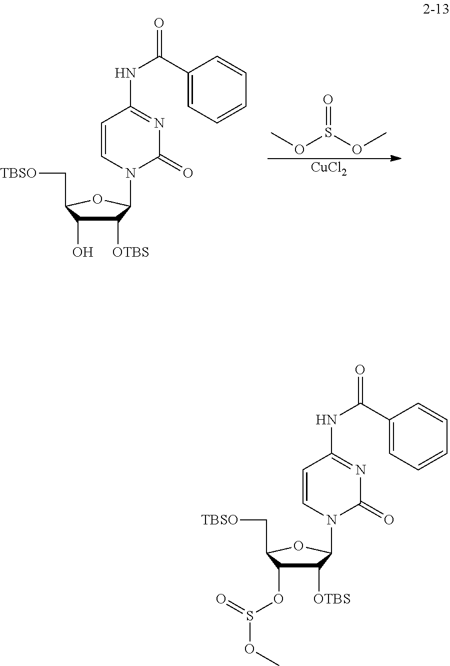

### (2R,3R,4R,5R)-5-(4-benzamido-2-oxopyrimidin-1(2H)-yl)-4-((tert-butyldimethylsilyl)oxy)-2-(((tert-butyldimethylsilyl)oxy)methyl)tetrahydrofuran-3-yl methyl sulfite

Same procedure as compound 1-13.

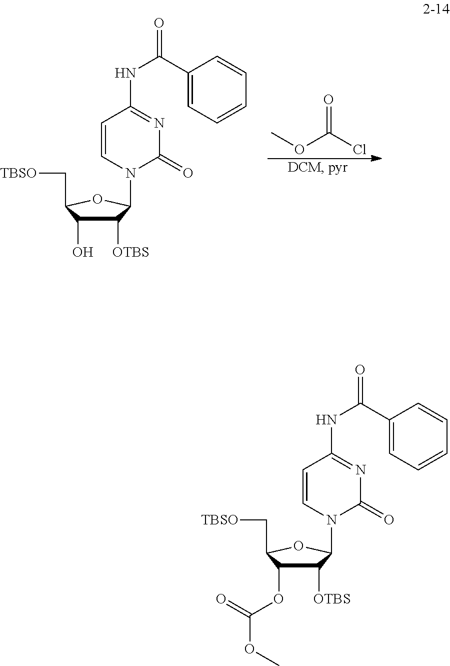

### (2R,3R,4R,5R)-5-(4-benzamido-2-oxopyrimidin-1(2H)-yl)-4-((tert-butyldimethylsilyl)oxy)-2-(((tert-butyldimethylsilyl)oxy)methyl)tetrahydrofuran-3-yl methyl carbonate

Same procedure as compound 1-14.

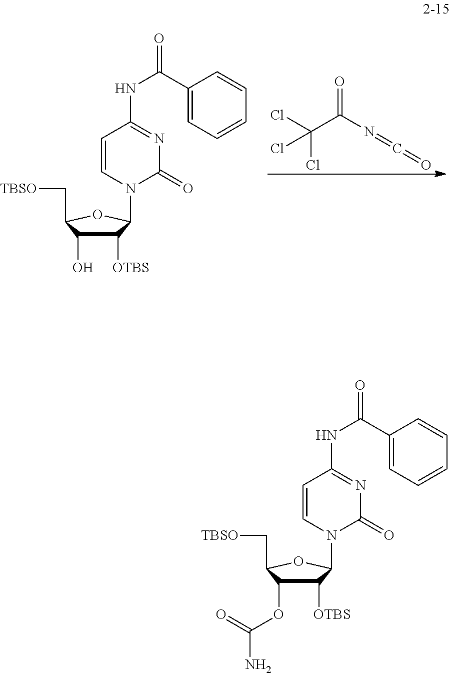

### (2R,3R,4R,5R)-5-(4-benzamido-2-oxopyrimidin-1(2H)-yl)-4-((tert-butyldimethylsilyl)oxy)-2-(((tert-butyldimethylsilyl)oxy)methyl)tetrahydrofuran-3-yl carbamate

Same procedure as compound 1-15.

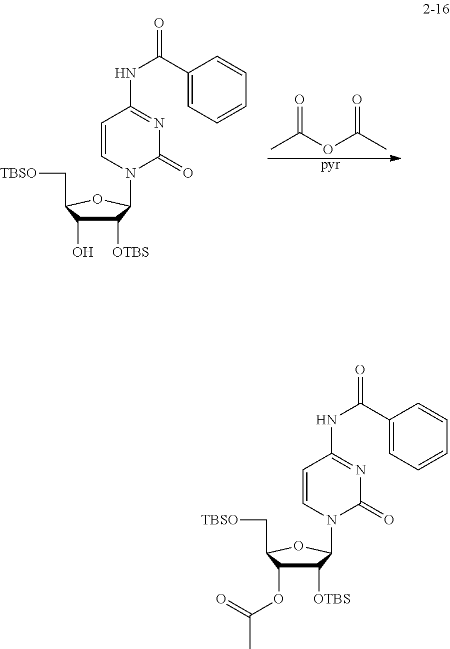

### (2R,3R,4R,5R)-5-(4-benzamido-2-oxopyrimidin-1(2H)-yl)-4-((tert-butyldimethylsilyl)oxy)-2-(((tert-butyldimethylsilyl)oxy)methyl)tetrahydrofuran-3-yl acetate

Same procedure as compound 1-16.

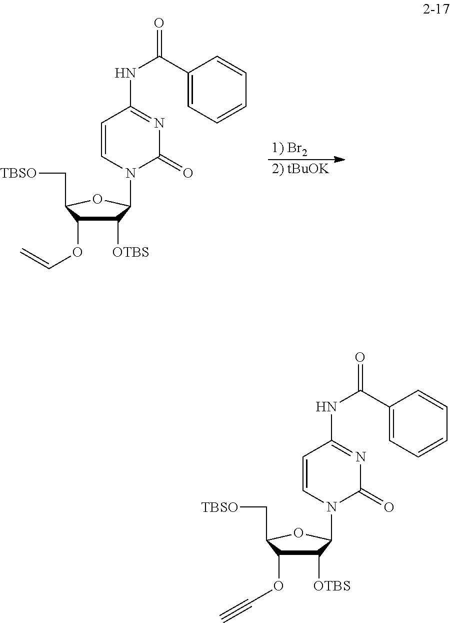

### N-(1-((2R,3R,4R,5R)-3-((tert-butyldimethylsilyl)oxy)-5-(((tert-butyldimethylsilyl)oxy)methyl)-4-(ethynyloxy)tetrahydrofuran-2-yl)-2-oxo-1,2-dihydropyrimidin-4-yl)benzamide

Same procedure as compound 1-17.

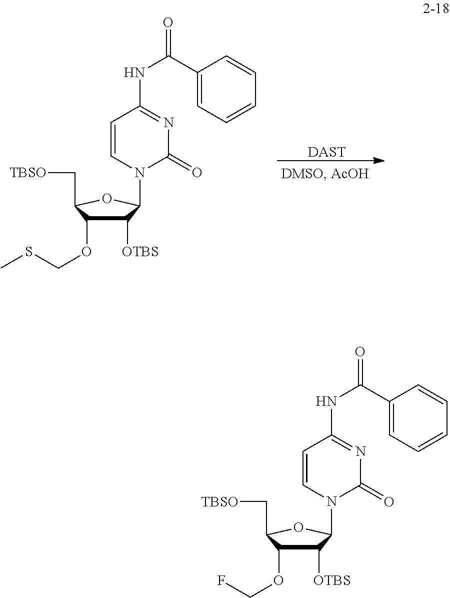

### N-(1-((2R,3R,4R,5R)-3-((tert-butyldimethylsilyl)oxy)-5-(((tert-butyldimethylsilyl)oxy)methyl)-4-(fluoromethoxy)tetrahydrofuran-2-yl)-2-oxo-1,2-dihydropyrimidin-4-yl)benzamide

Same procedure as compound 1-18.

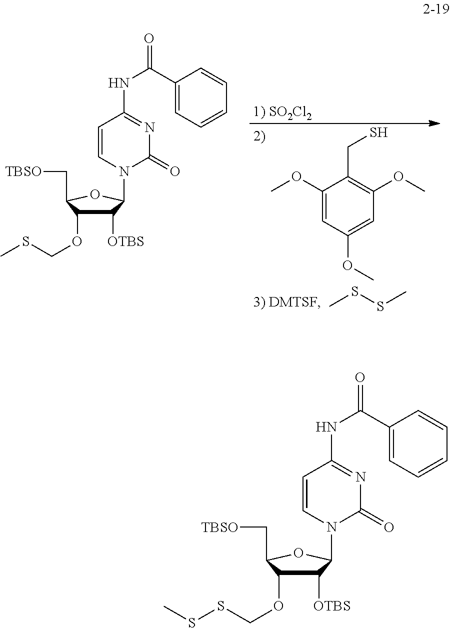

### N-(1-((2R,3R,4R,5R)-3-((tert-butyldimethylsilyl)oxy)-5-(((tert-butyldimethylsilyl)oxy)methyl)-4-((methyldisulfaneyl)methoxy)tetrahydrofuran-2-yl)-2-oxo-1,2-dihydropyrimidin-4-yl)benzamide

Same procedure as compound 1-19.

### Example 5: A Common Adenine Intermediate was Synthesized as Follows

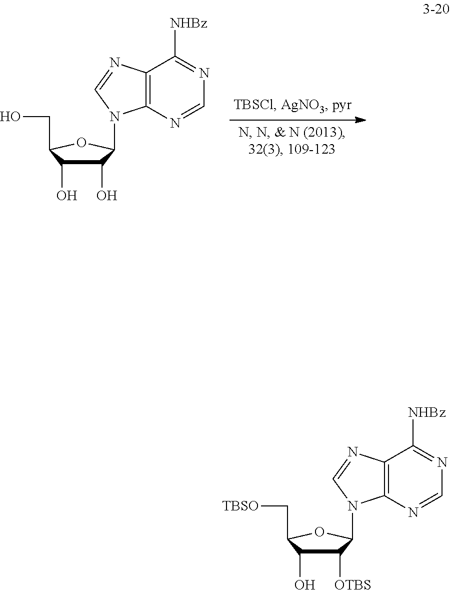

### N-(9-((2R,3R,4R,5R)-3-((tert-butyldimethylsilyl)oxy)-5-(((tert-butyldimethylsilyl)oxy)methyl)-4-hydroxytetrahydrofuran-2-yl)-9H-purin-6-yl)benzamide

Same procedure as compound 1-20.

### Example 6: Analogs (3-1 Through 3-19 Above) were Prepared Using the Following Methods

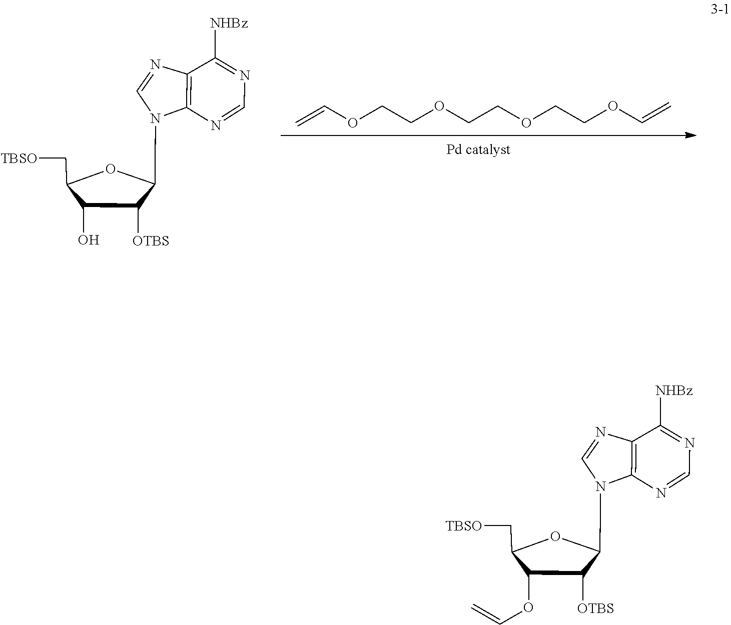

### N-(9-((2R,3R,4R,5R)-3-((tert-butyldimethylsilyl)oxy)-5-(((tert-butyldimethylsilyl)oxy)methyl)-4-(vinyloxy)tetrahydrofuran-2-yl)-9H-purin-6-yl)benzamide

Same procedure as compound 1-1.

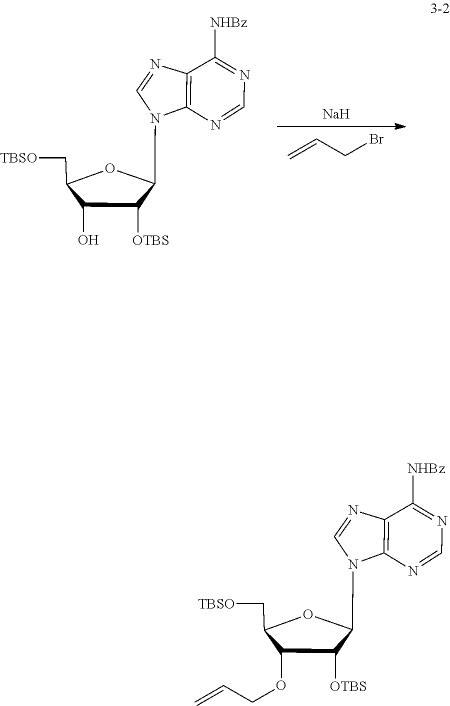

### N-(9-((2R,3R,4R,5R)-4-(allyloxy)-3-((tert-butyldimethylsilyl)oxy)-5-(((tert-butyldimethylsilyl)oxy)methyl)tetrahydrofuran-2-yl)-9H-purin-6-yl)benzamide

Same procedure as compound 1-2.

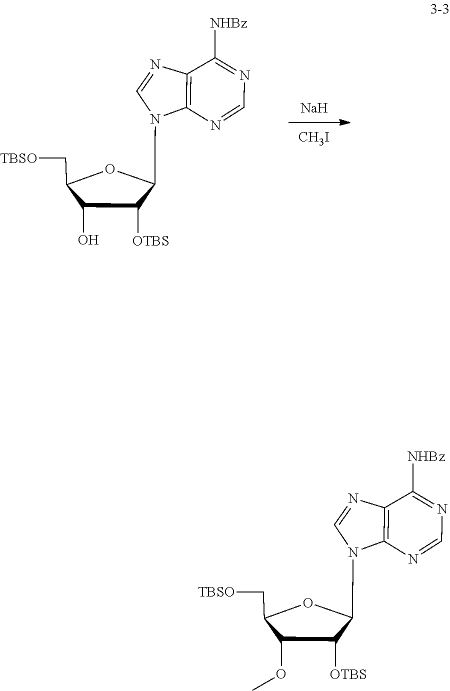

### N-(9-((2R,3R,4R,5R)-3-((tert-butyldimethylsilyl)oxy)-5-(((tert-butyldimethylsilyl)oxy)methyl)-4-methoxytetrahydrofuran-2-yl)-9H-purin-6-yl)benzamide

Same procedure as compound 1-3.

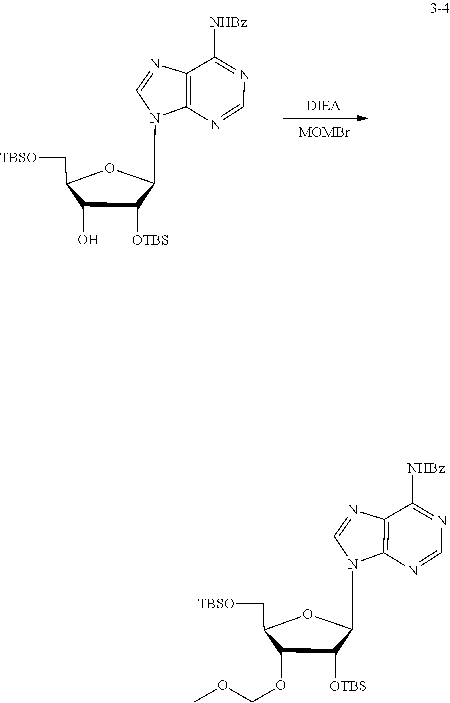

### N-(9-((2R,3R,4R,5R)-3-((tert-butyldimethylsilyl)oxy)-5-(((tert-butyldimethylsilyl)oxy)methyl)-4-(methoxymethoxy)tetrahydrofuran-2-yl)-9H-purin-6-yl)benzamide

Same procedure as compound 1-4.

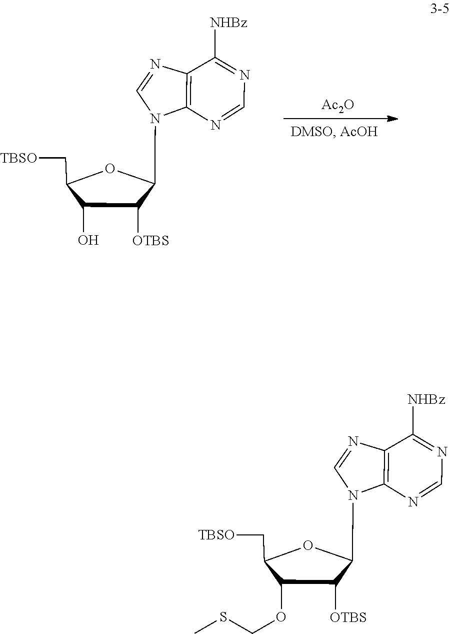

### N-(9-((2R,3R,4R,5R)-3-((tert-butyldimethylsilyl)oxy)-5-(((tert-butyldimethylsilyl)oxy)methyl)-4-((methylthio)methoxy)tetrahydrofuran-2-yl)-9H-purin-6-yl)benzamide

Same procedure as compound 1-5.

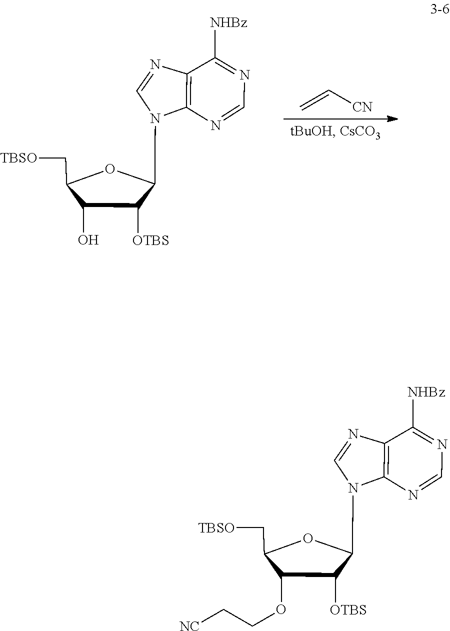

### N-(9-((2R,3R,4R,5R)-3-((tert-butyldimethylsilyl)oxy)-5-(((tert-butyldimethylsilyl)oxy)methyl)-4-(2-cyanoethoxy)tetrahydrofuran-2-yl)-9H-purin-6-yl)benzamide

Same procedure as compound 1-6.

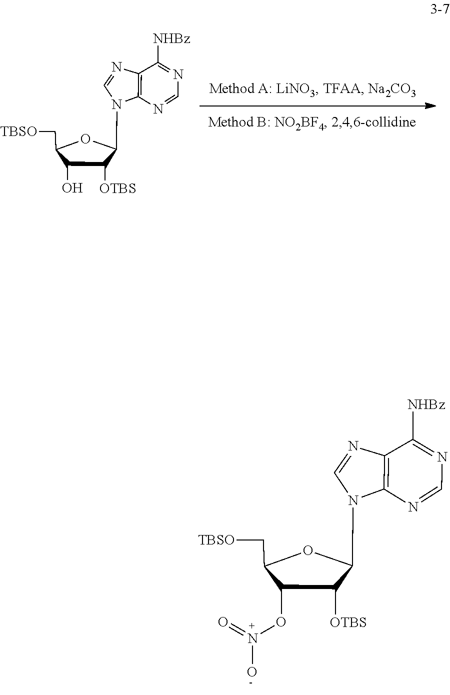

### (2R,3R,4R,5R)-5-(6-benzamido-9H-purin-9-yl)-4-((tert-butyldimethylsilyl)oxy)-2-(((tert-butyldimethylsilyl)oxy)methyl)tetrahydrofuran-3-yl nitrate

Same procedure as compound 1-7.

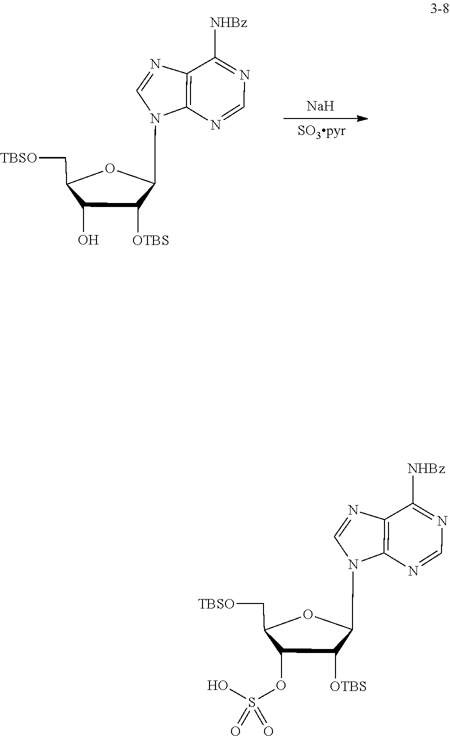

### (2R,3R,4R,5R)-5-(6-benzamido-9H-purin-9-yl)-4-((tert-butyldimethylsilyl)oxy)-2-(((tert-butyldimethylsilyl)oxy)methyl)tetrahydrofuran-3-yl hydrogen sulfate

Same procedure as compound 1-8.

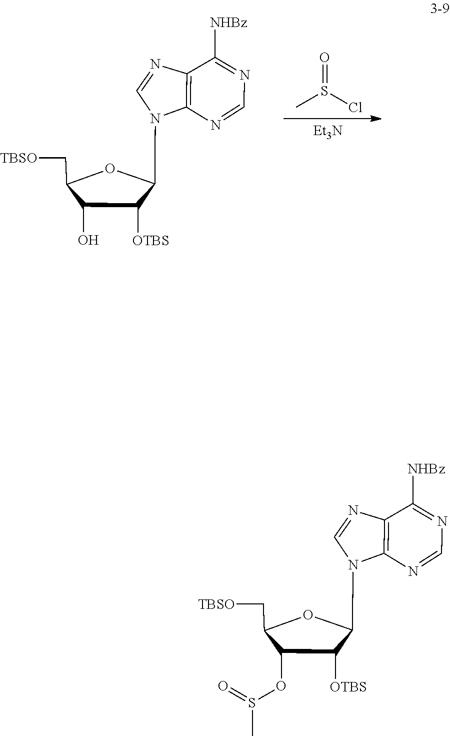

### (2R,3R,4R,5R)-5-(6-benzamido-9H-purin-9-yl)-4-((tert-butyldimethylsilyl)oxy)-2-(((tert-butyldimethylsilyl)oxy)methyl)tetrahydrofuran-3-yl methanesulfinate

Same procedure as compound 1-9.

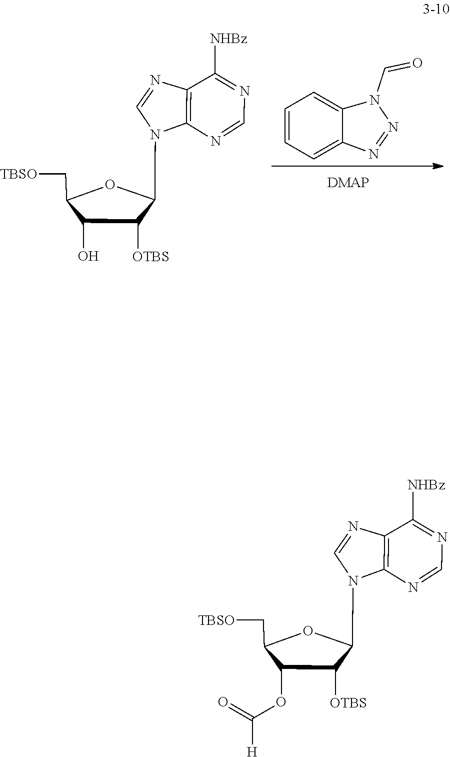

### (2R,3R,4R,5R)-5-(6-benzamido-9H-purin-9-yl)-4-((tert-butyldimethylsilyl)oxy)-2-(((tert-butyldimethylsilyl)oxy)methyl)tetrahydrofuran-3-yl formate

Same procedure as compound 1-10.

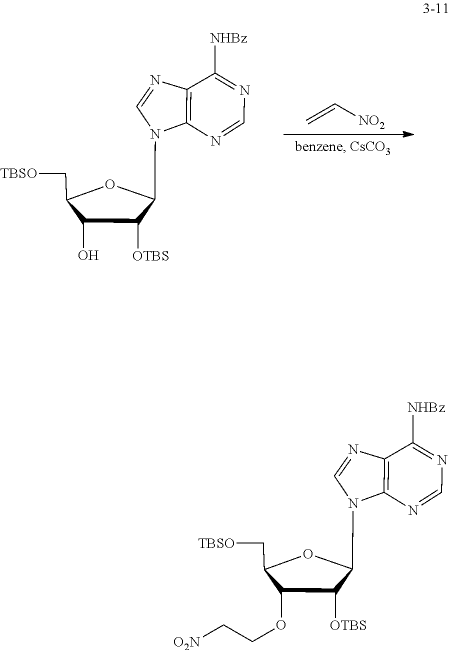

### N-(9-((2R,3R,4R,5R)-3-((tert-butyldimethylsilyl)oxy)-5-(((tert-butyldimethylsilyl)oxy)methyl)-4-(2-nitroethoxy)tetrahydrofuran-2-yl)-9H-purin-6-yl)benzamide

Same procedure as compound 1-11.

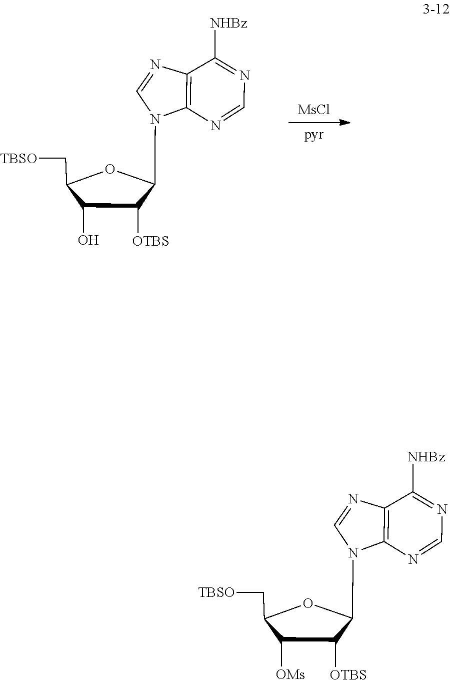

### (2R,3R,4R,5R)-5-(6-benzamido-9H-purin-9-yl)-4-((tert-butyldimethylsilyl)oxy)-2-(((tert-butyldimethylsilyl)oxy)methyl)tetrahydrofuran-3-yl methanesulfonate

Same procedure as compound 1-12.

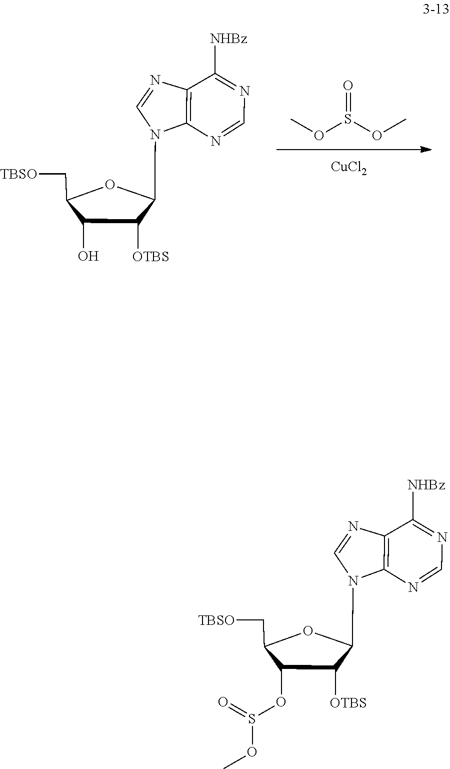

### (2R,3R,4R,5R)-5-(6-benzamido-9H-purin-9-yl)-4-((tert-butyldimethylsilyl)oxy)-2-(((tert-butyldimethylsilyl)oxy)methyl)tetrahydrofuran-3-yl methyl sulfate

Same procedure as compound 1-13.

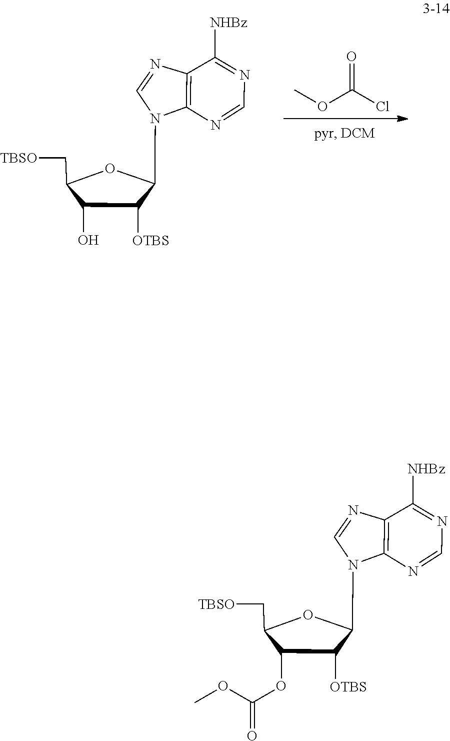

### (2R,3R,4R,5R)-5-(6-benzamido-9H-purin-9-yl)-4-((tert-butyldimethylsilyl)oxy)-2-(((tert-butyldimethylsilyl)oxy)methyl)tetrahydrofuran-3-yl methyl carbonate

Same procedure as compound 1-14.

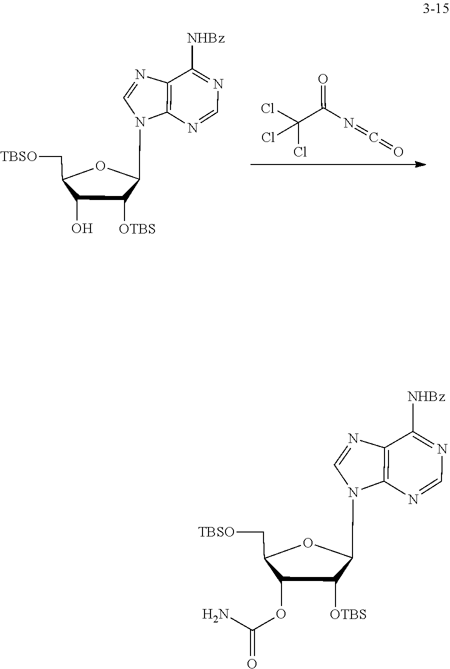

### (2R,3R,4R,5R)-5-(6-benzamido-9H-purin-9-yl)-4-((tert-butyldimethylsilyl)oxy)-2-(((tert-butyldimethylsilyl)oxy)methyl)tetrahydrofuran-3-yl carbamate

Same procedure as compound 1-15.

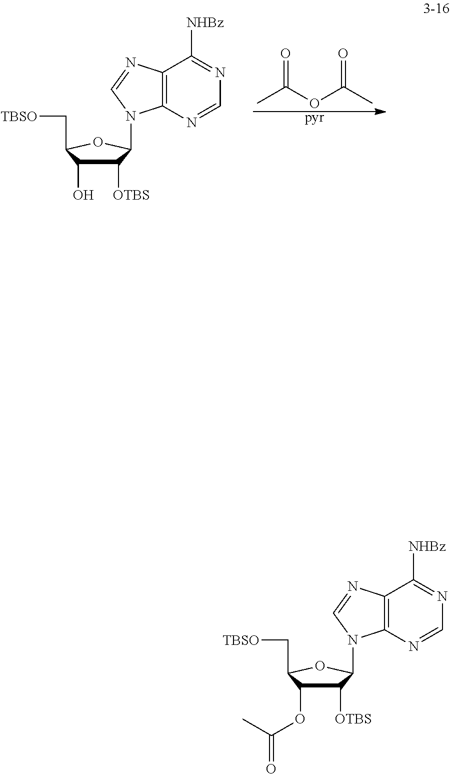

### (2R,3R,4R,5R)-5-(6-benzamido-9H-purin-9-yl)-4-((tert-butyldimethylsilyl)oxy)-2-(((tert-butyldimethylsilyl)oxy)methyl)tetrahydrofuran-3-yl acetate

Same procedure as compound 1-16.

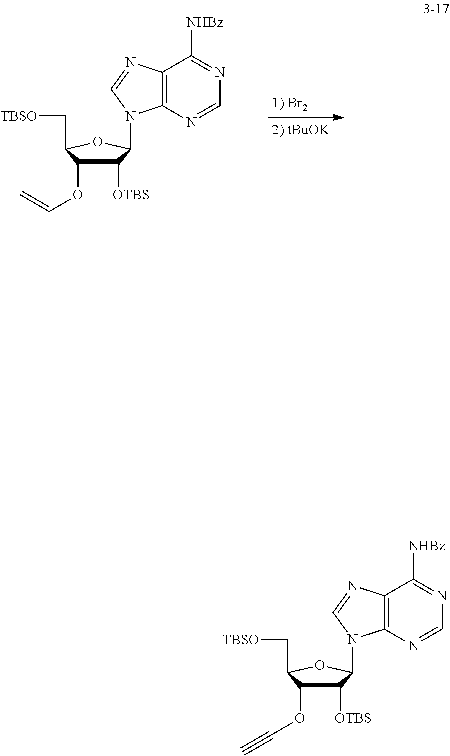

### N-(9-((2R,3R,4R,5R)-3-((tert-butyldimethylsilyl)oxy)-5-(((tert-butyldimethylsilyl)oxy)methyl)-4-(ethynyloxy)tetrahydrofuran-2-yl)-9H-purin-6-yl)benzamide

Same procedure as compound 1-17.

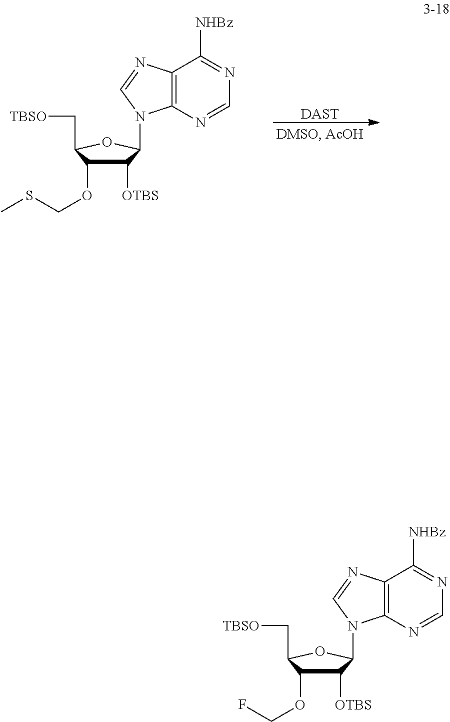

### N-(9-((2R,3R,4R,5R)-3-((tert-butyldimethylsilyl)oxy)-5-(((tert-butyldimethylsilyl)oxy)methyl)-4-(fluoromethoxy)tetrahydrofuran-2-yl)-9H-purin-6-yl)benzamide

Same procedure as compound 1-18.

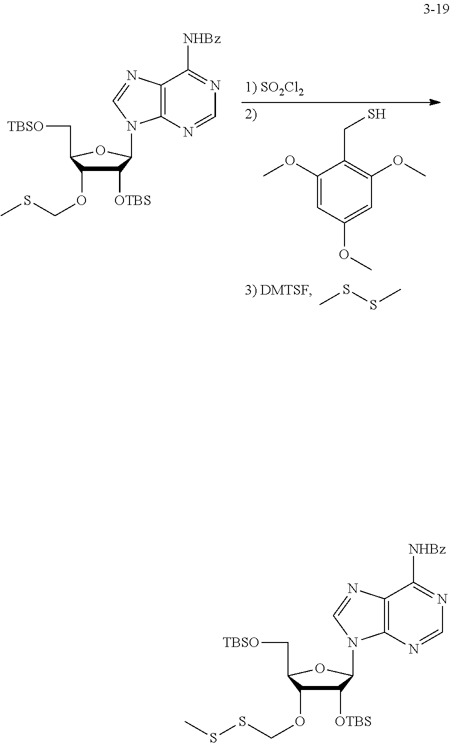

### N-(9-((2R,3R,4R,5R)-3-((tert-butyldimethylsilyl)oxy)-5-(((tert-butyldimethylsilyl)oxy)methyl)-4-((methyldisulfaneyl)methoxy)tetrahydrofuran-2-yl)-9H-purin-6-yl)benzamide

Same procedure as compound 1-19.

### Example 7: A Common Guanine Intermediate was Synthesized as Follows

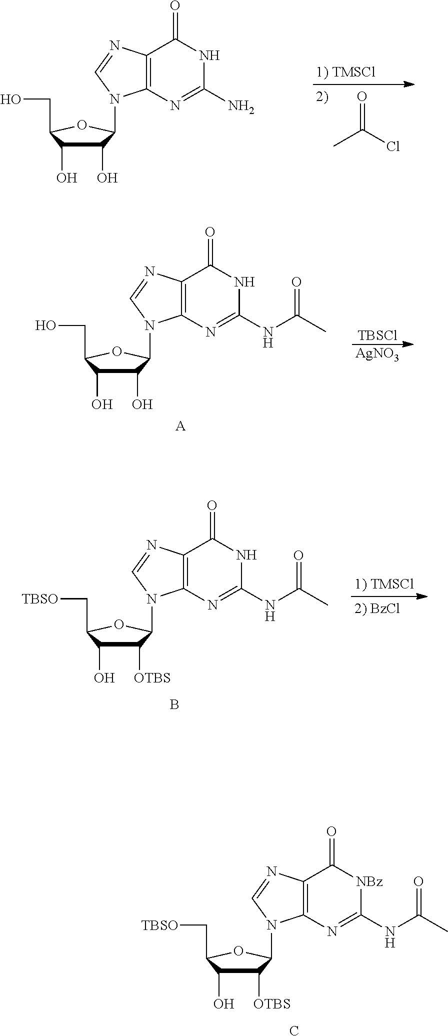

Compound A: To a suspension of 2-amino-9-((2R,3R,4S,5R)-3,4-dihydroxy-5-(hydroxymethyl)tetrahydrofuran-2-yl)-1,9-dihydro-6H-purin-6-one (1 equiv.), which was rendered anhydrous by co-evaporation with pyridine (10 mL×3), in DCM at 0° C. under Ar was added TMSCl (9 equiv.). The ice bath was removed, and the reaction was continuously stirred at rt. After 2 h, the reaction was cooled to 0° C. and to it was added acetyl chloride (1.1 equiv.) over 10 min and allowed to stir for 1.5 h. The reaction was quenched with MeOH (20 mL) and allowed to stir overnight for complete desilylation. The crude was evaporated to an oil and re-suspended in MeOH (100 mL) while stirring at 0° C. for 1 h. The precipitated white solid was vacuum filtered and dried over high vacuum.

Compound B: To a suspension of N-(9-((2R,3R,4S,5R)-3,4-dihydroxy-5-(hydroxymethyl)tetrahydrofuran-2-yl)-6-oxo-6,9-dihydro-1H-purin-2-yl)acetamide (1 equiv.) in THF at rt under Ar was added anhydrous pyridine (7 equiv.) followed by silver nitrate (3 equiv.) which was stirred for 48 h. The reaction was quenched with ethanol (10 mL) and the mixture was vacuum filtered over celite. The filtrate was concentrated, re-dissolved in EtOAc and sequentially washed with water (25 mL×1) and brine (25 mL×1). The organic phase was dried over Na₂SO₄, filtered, and purified by chromatography (0-5% MeOH in DCM) to afford N-(9-((2R,3R,4R,5R)-3-((tert-butyldimethylsilyl)oxy)-5-(((tert-butyldimethylsilyl)oxy)methyl)-4-hydroxytetrahydrofuran-2-yl)-6-oxo-6,9-dihydro-1H-purin-2-yl)acetamide.

Compound C: To a solution of N-(9-((2R,3R,4R,5R)-3-((tert-butyldimethylsilyl)oxy)-5-(((tert-butyldimethylsilyl)oxy)methyl)-4-hydroxytetrahydrofuran-2-yl)-6-oxo-6,9-dihydro-1H-purin-2-yl)acetamide (1 equiv.) in anhydrous pyridine at rt under Ar was added TMSCI (2 equiv.) and allowed to stir for 1 h. The reaction mixture was cooled to 0° C. and to it was added a solution of benzoyl chloride in DCM (1.1 equiv.) dropwise via an addition funnel. After 2 h, the reaction was quenched with MeOH (20 mL) and stirred overnight for complete desilylation. The crude was concentrated to an oil and directly subjected to chromatography (0-5% MeOH in DCM) to afford N-(1-benzoyl-9-((2R,3R,4R,5R)-3-((tert-butyldimethylsilyl)oxy)-5-(((tert-butyldimethylsilyl)oxy)methyl)-4-hydroxytetrahydrofuran-2-yl)-6-oxo-6,9-dihydro-1H-purin-2-yl)acetamide.

### Example 8: Analogs (4-1 Through 4-19 Above) were Prepared Using the Following Methods

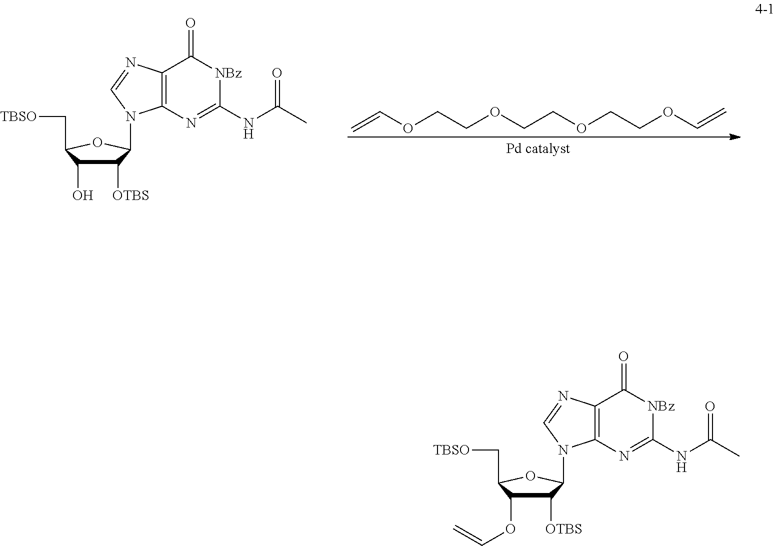

### N-(1-benzoyl-9-((2R,3R,4R,5R)-3-((tert-butyldimethylsilyl)oxy)-5-(((tert-butyldimethylsilyl)oxy)methyl)-4-(vinyloxy)tetrahydrofuran-2-yl)-6-oxo-6,9-dihydro-1H-purin-2-yl)acetamide

Same procedure as compound 1-1.

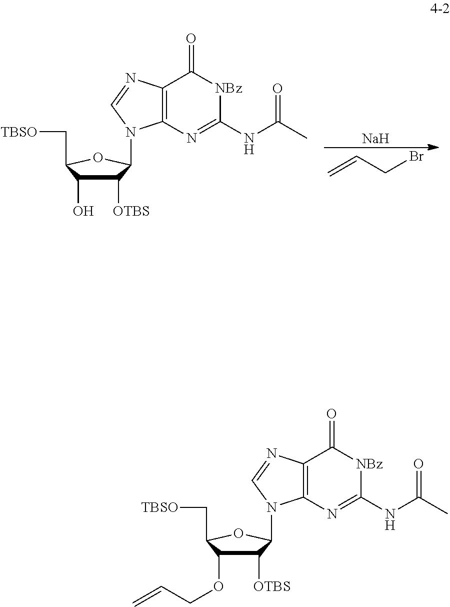

### N-(9-((2R,3R,4R,5R)-4-(allyloxy)-3-((tert-butyldimethylsilyl)oxy)-5-(((tert-butyldimethylsilyl)oxy)methyl)tetrahydrofuran-2-yl)-1-benzoyl-6-oxo-6,9-dihydro-1H-purin-2-yl)acetamide

Same procedure as compound 1-2.

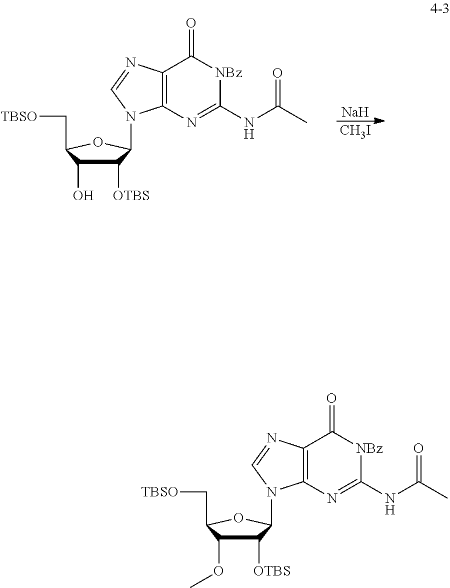

### N-(1-benzoyl-9-((2R,3R,4R,5R)-3-((tert-butyldimethylsilyl)oxy)-5-(((tert-butyldimethylsilyl)oxy)methyl)-4-methoxytetrahydrofuran-2-yl)-6-oxo-6,9-dihydro-1H-purin-2-yl)acetamide

Same procedure as compound 1-3.

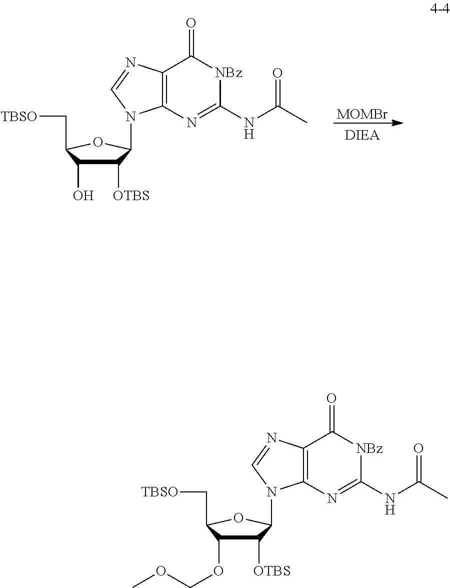

### N-(1-benzoyl-9-((2R,3R,4R,5R)-3-((tert-butyldimethylsilyl)oxy)-5-(((tert-butyldimethylsilyl)oxy)methyl)-4-(methoxymethoxy)tetrahydrofuran-2-yl)-6-oxo-6,9-dihydro-1H-purin-2-yl)acetamide

Same procedure as compound 1-4.

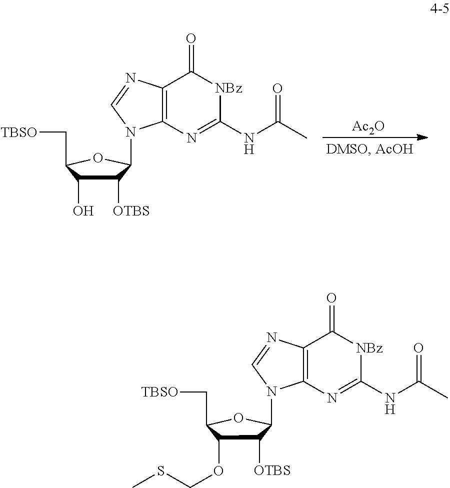

### N-(1-benzoyl-9-((2R,3R,4R,5R)-3-((tert-butyldimethylsilyl)oxy)-5-(((tert-butyldimethylsilyl)oxy)methyl)-4-((methylthio)methoxy)tetrahydrofuran-2-yl)-6-oxo-6,9-dihydro-1H-purin-2-yl)acetamide

Same procedure as compound 1-5.

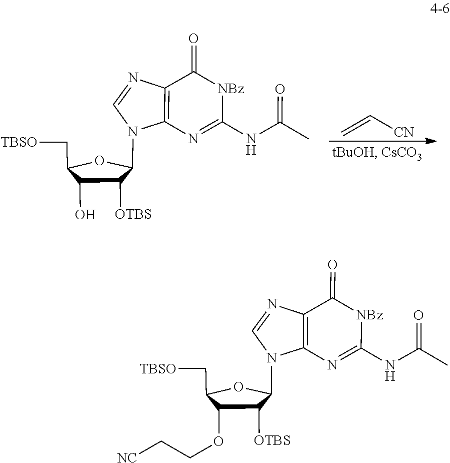

### N-(1-benzoyl-9-((2R,3R,4R,5R)-3-((tert-butyldimethylsilyl)oxy)-5-(((tert-butyldimethylsilyl)oxy)methyl)-4-(2-cyanoethoxy)tetrahydrofuran-2-yl)-6-oxo-6,9-dihydro-1H-purin-2-yl)acetamide

Same procedure as compound 1-6.

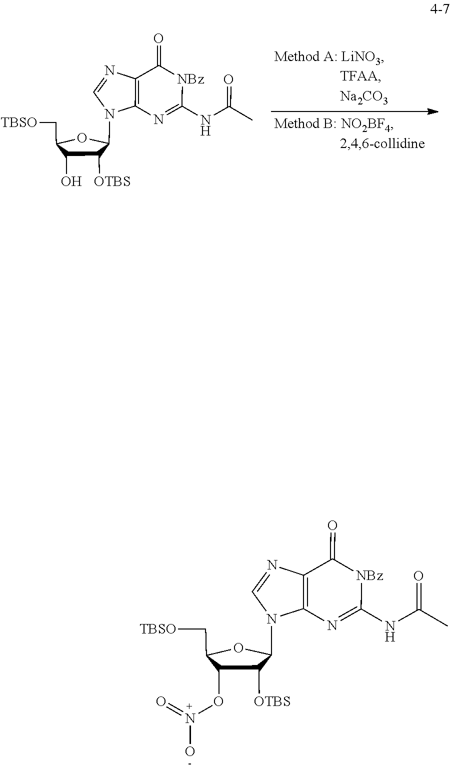

### (2R,3R,4R,5R)-5-(2-acetamido-1-benzoyl-6-oxo-1,6-dihydro-9H-purin-9-yl)-4-((tert-butyldimethylsilyl)oxy)-2-(((tert-butyldimethylsilyl)oxy)methyl)tetrahydrofuran-3-yl nitrate

Same procedure as compound 1-7.

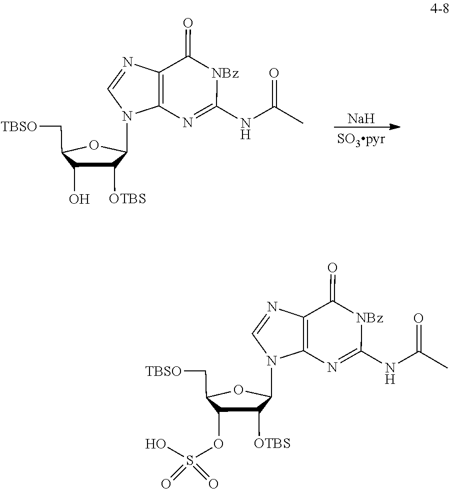

### (2R,3R,4R,5R)-5-(2-acetamido-1-benzoyl-6-oxo-1,6-dihydro-9H-purin-9-yl)-4-((tert-butyldimethylsilyl)oxy)-2-(((tert-butyldimethylsilyl)oxy)methyl)tetrahydrofuran-3-yl hydrogen sulfate

Same procedure as compound 1-8.

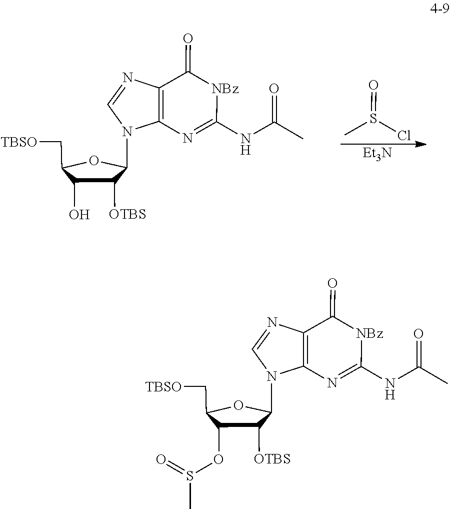

### (2R,3R,4R,5R)-5-(2-acetamido-1-benzoyl-6-oxo-1,6-dihydro-9H-purin-9-yl)-4-((tert-butyldimethylsilyl)oxy)-2-(((tert-butyldimethylsilyl)oxy)methyl)tetrahydrofuran-3-yl methanesulfinate

Same procedure as compound 1-9.

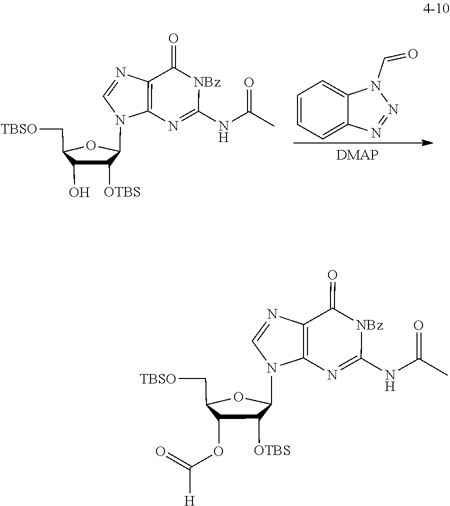

### (2R,3R,4R,5R)-5-(2-acetamido-1-benzoyl-6-oxo-1,6-dihydro-9H-purin-9-yl)-4-((tert-butyldimethylsilyl)oxy)-2-(((tert-butyldimethylsilyl)oxy)methyl)tetrahydrofuran-3-yl formate

Same procedure as compound 1-10.

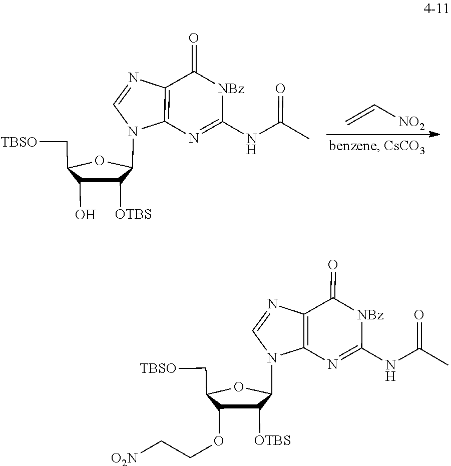

### N-(1-benzoyl-9-((2R,3R,4R,5R)-3-((tert-butyldimethylsilyl)oxy)-5-(((tert-butyldimethylsilyl)oxy)methyl)-4-(2-nitroethoxy)tetrahydrofuran-2-yl)-6-oxo-6,9-dihydro-1H-purin-2-yl)acetamide

Same procedure as compound 1-11.

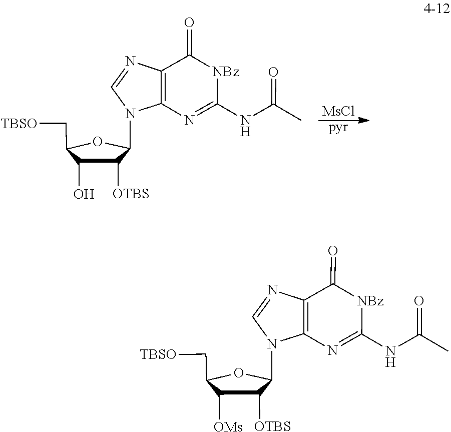

### (2R,3R,4R,5R)-5-(2-acetamido-1-benzoyl-6-oxo-1,6-dihydro-9H-purin-9-yl)-4-((tert-butyldimethylsilyl)oxy)-2-(((tert-butyldimethylsilyl)oxy)methyl)tetrahydrofuran-3-yl methanesulfonate

Same procedure as compound 1-12.

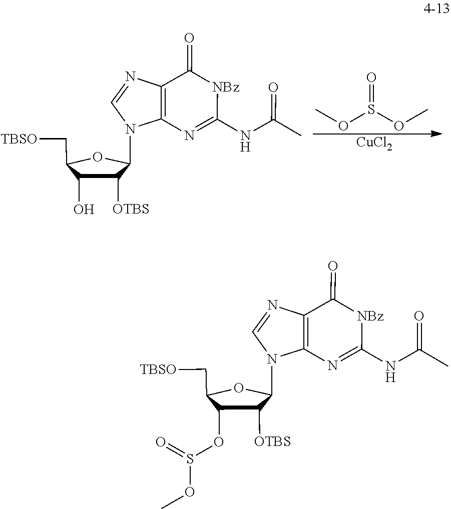

### (2R,3R,4R,5R)-5-(2-acetamido-1-benzoyl-6-oxo-1,6-dihydro-9H-purin-9-yl)-4-((tert-butyldimethylsilyl)oxy)-2-(((tert-butyldimethylsilyl)oxy)methyl)tetrahydrofuran-3-yl methyl sulfate

Same procedure as compound 1-13.

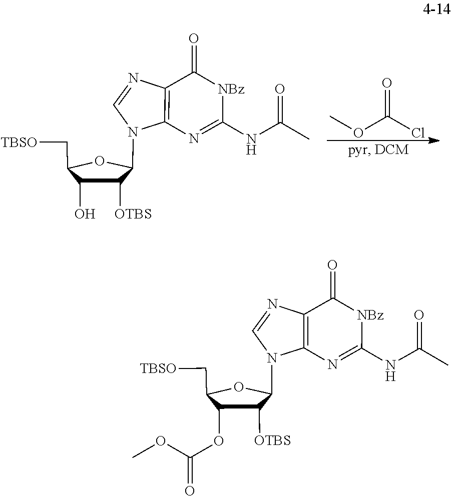

### (2R,3R,4R,5R)-5-(2-acetamido-1-benzoyl-6-oxo-1,6-dihydro-9H-purin-9-yl)-4-((tert-butyldimethylsilyl)oxy)-2-(((tert-butyldimethylsilyl)oxy)methyl)tetrahydrofuran-3-yl methyl carbonate

Same procedure as compound 1-14.

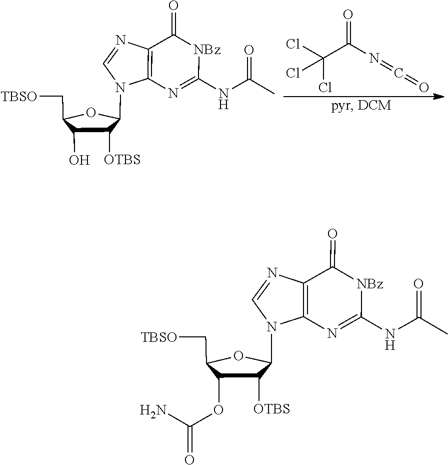

### (2R,3R,4R,5R)-5-(2-acetamido-1-benzoyl-6-oxo-1,6-dihydro-9H-purin-9-yl)-4-((tert-butyldimethylsilyl)oxy)-2-(((tert-butyldimethylsilyl)oxy)methyl)tetrahydrofuran-3-yl carbamate

Same procedure as compound 1-15.

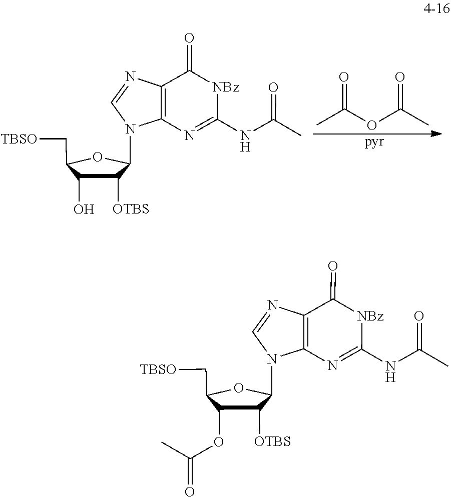

### (2R,3R,4R,5R)-5-(2-acetamido-1-benzoyl-6-oxo-1,6-dihydro-9H-purin-9-yl)-4-((tert-butyldimethylsilyl)oxy)-2-(((tert-butyldimethylsilyl)oxy)methyl)tetrahydrofuran-3-yl acetate

Same procedure as compound 1-16.

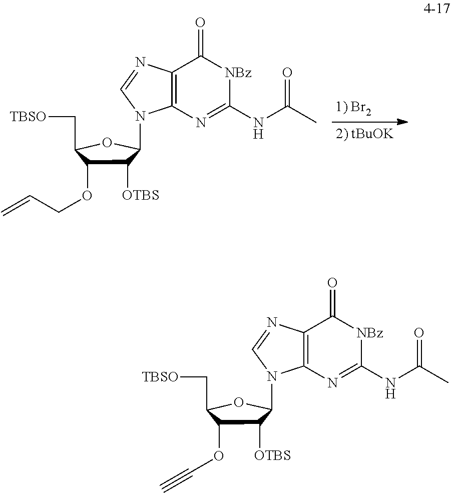

### N-(1-benzoyl-9-((2R,3R,4R,5R)-3-((tert-butyldimethylsilyl)oxy)-5-(((tert-butyldimethylsilyl)oxy)methyl)-4-(ethynyloxy)tetrahydrofuran-2-yl)-6-oxo-6,9-dihydro-1H-purin-2-yl)acetamide

Same procedure as compound 1-17.

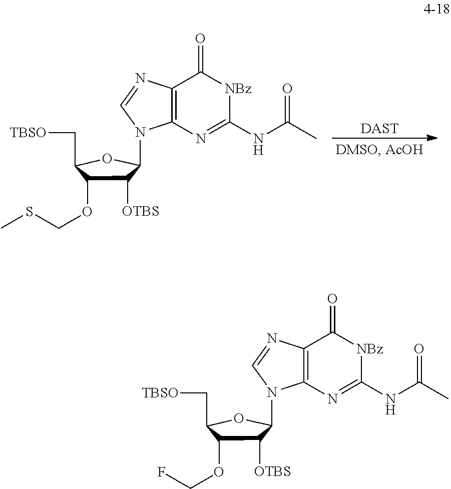

### N-(1-benzoyl-9-((2R,3R,4R,5R)-3-((tert-butyldimethylsilyl)oxy)-5-(((tert-butyldimethylsilyl)oxy)methyl)-4-(fluoromethoxy)tetrahydrofuran-2-yl)-6-oxo-6,9-dihydro-1H-purin-2-yl)acetamide

Same procedure as compound 1-18.

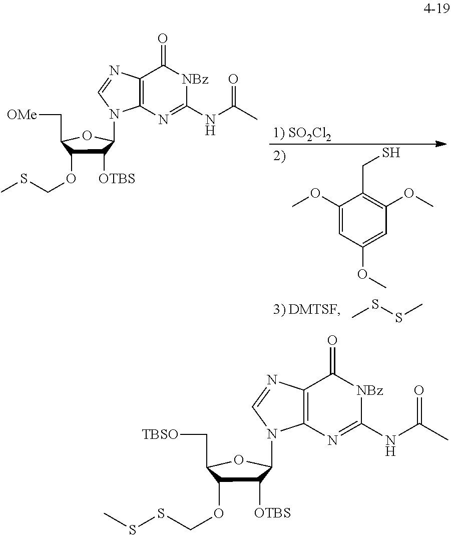

### N-(1-benzoyl-9-((2R,3R,4R,5R)-3-((tert-butyldimethylsilyl)oxy)-5-(((tert-butyldimethylsilyl)oxy)methyl)-4-((methyldisulfaneyl)methoxy)tetrahydrofuran-2-yl)-6-oxo-6,9-dihydro-1H-purin-2-yl)acetamide

Same procedure as compound 1-19.

### Example 9: Synthesis of Reagent A

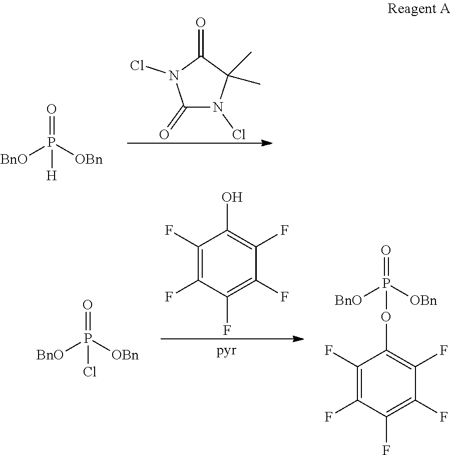 [SMILES: O=[PH](OCc1ccccc1)OCc1ccccc1 ; CC1(C)C(=O)N(Cl)C(=O)N1Cl ; O=P(Cl)(OCc1ccccc1)OCc1ccccc1 ; Oc1c(F)c(F)c(F)c(F)c1F ; O=P(OCc1ccccc1)(OCc1ccccc1)Oc1c(F)c(F)c(F)c(F)c1F]

To a solution of dibenzyl phosphonate (1.25 Eq) in acetonitrile at rt under Ar was added 1,3-dichloro-5,5-dimethylimidazolidine-2,4-dione (0.63 Eq) as a solid and allowed to stir for 1 h. To an ice-cooled solution of pentafluorophenol (1.25 Eq) and pyridine (1.25 Eq) in acetonitrile was then added the crude dibenzyl chlorophosphite (see above) via syringe over 5 min and allowed to stir for 1 h at 0° C. The reaction was removed from the ice-bath and continued to stir at rt for another hour. The contents were diluted in EtOAc and washed with water (×1) followed by brine (×1). The organic phase was dried over Na₂SO₄, filtered, and purified by chromatography (0-20% EtOAc in hexanes) to afford Reagent A.

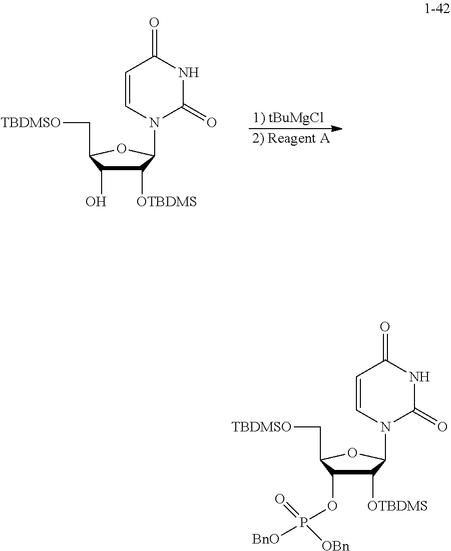

To a solution of 1-((2R,3R,4R,5R)-3-((tert-butyldimethylsilyl)oxy)-5-(((tert-butyldimethylsilyl)oxy)methyl)-4-((methylthio)methoxy)tetrahydrofuran-2-yl)pyrimidine-2,4(1H,3H)-dione (1 equiv.) in THF at 0° C. under Ar was added tBuMgCl (1.0M, 2 equiv.) and stirred for 50 min. The ice-bath was removed and the reaction was allowed to stir at ambient temperature for an additional 90 min. To the reaction mixture was added a solution of Reagent A (1.1 equiv.) in THE and allowed to stir for 2 h. The contents were diluted in EtOAc and washed with water (×1) followed by brine (×1). The organic phase was dried over Na₂SO₄, filtered, and purified by chromatography (0-40% EtOAc in hexanes) to afford target compound 1-42.

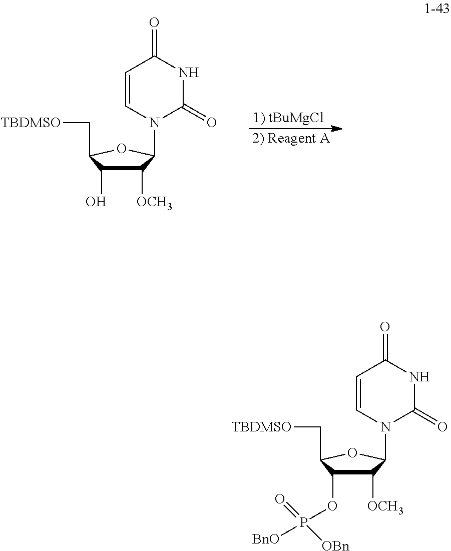

Starting material 1-((2R,3R,4R,5R)-5-(((tert-butyldimethylsilyl)oxy)methyl)-4-hydroxy-3-methoxytetrahydrofuran-2-yl)pyrimidine-2,4(1H,3H)-dione was obtained following patent US20120070411 A1 2012-03-22. Compound 1-43 was synthesized using a similar procedure as 1-42.

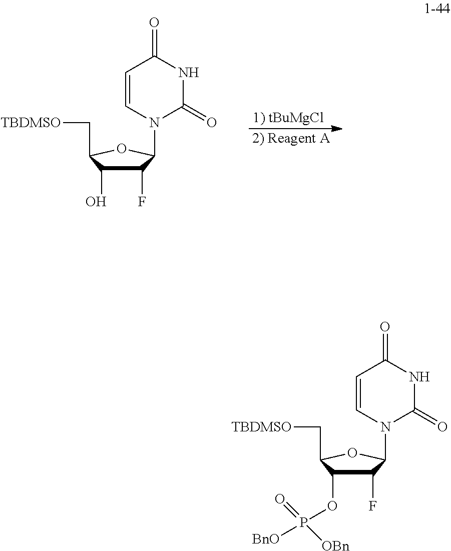

Starting material 1-((2R,3R,4R,5R)-5-(((tert-butyldimethylsilyl)oxy)methyl)-3-fluoro-4-hydroxytetrahydrofuran-2-yl)pyrimidine-2,4(1H,3H)-dione was obtained following patent WO2018045317 A1 2018-03-08. Compound 1-25 was synthesized using a similar procedure as 1-42

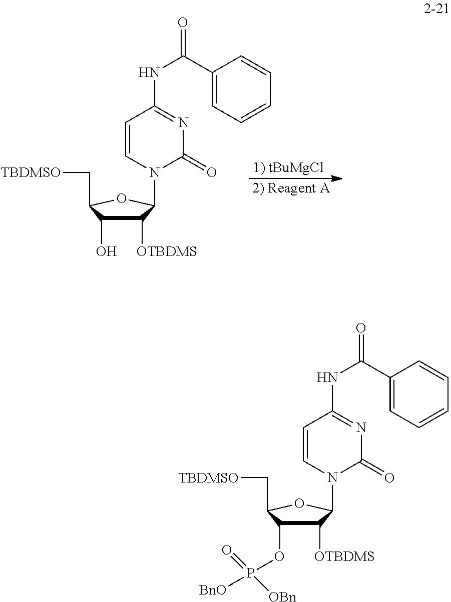

Same procedure as compound 1-42

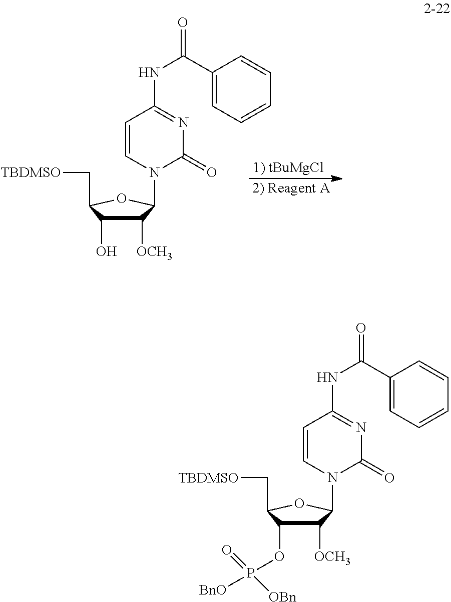

Starting material N-(1-((2R,3R,4R,5R)-5-(((tert-butyldimethylsilyl)oxy)methyl)-4-hydroxy-3-methoxytetrahydrofuran-2-yl)-2-oxo-1,2-dihydropyrimidin-4-yl)benzamide was obtained according to patent CN109053839 A 2018-12-21. Target compound 2-22 was synthesized using the same procedure as compound 2-21

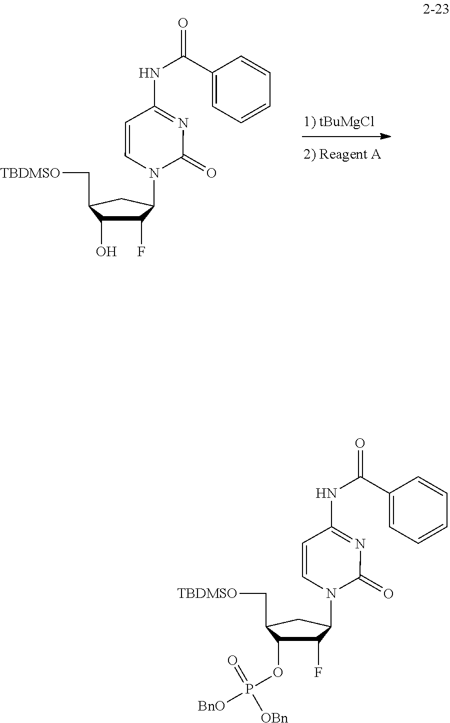

Starting material N-(1-((2R,3R,4R,5R)-5-(((tert-butyldimethylsilyl)oxy)methyl)-3-fluoro-4-hydroxytetrahydrofuran-2-yl)-2-oxo-1,2-dihydropyrimidin-4-yl)benzamide was obtained according to Nucleosides, Nucleotides & Nucleic Acids (2005), 24(5-7), 713-715. Target compound 2-23 was synthesized using the same procedure as compound 2-21X

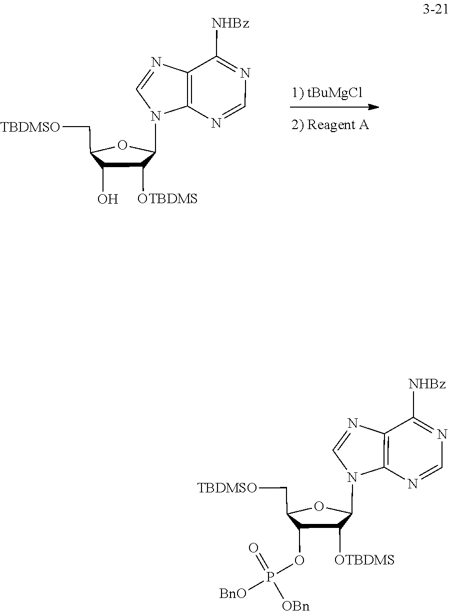

Same procedure as compound 1-42

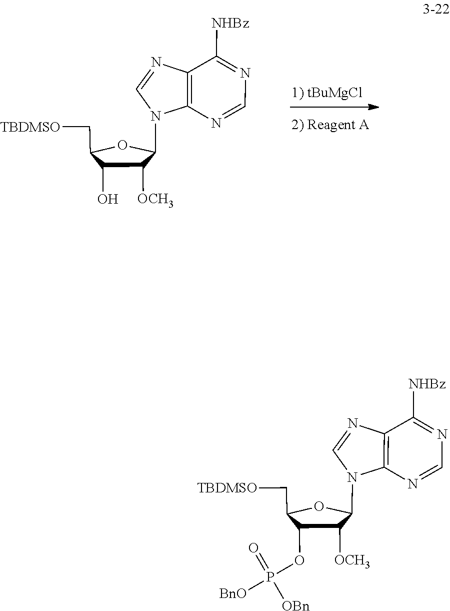

Same procedure as compound 3-21

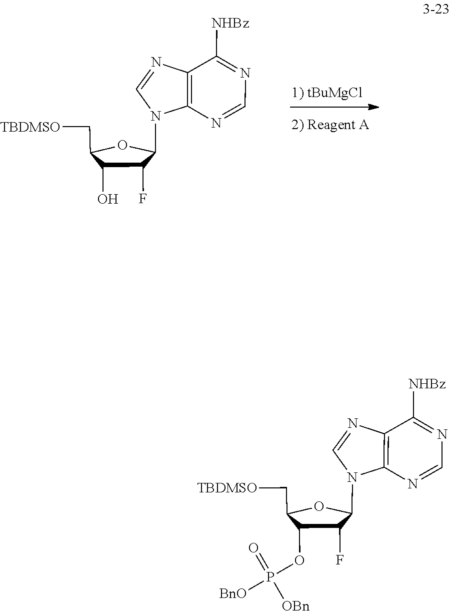

Same procedure as compound 3-21

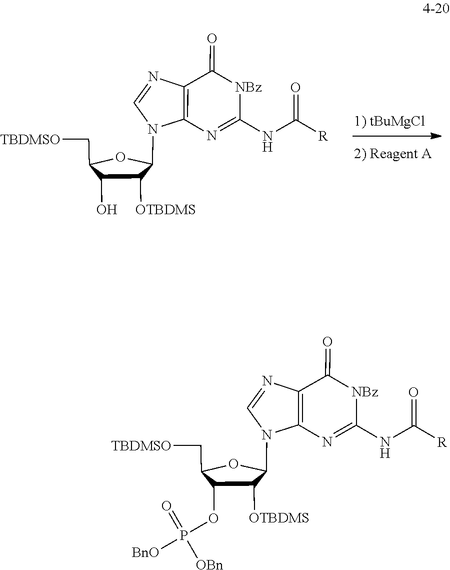

Same procedure as compound 1-42

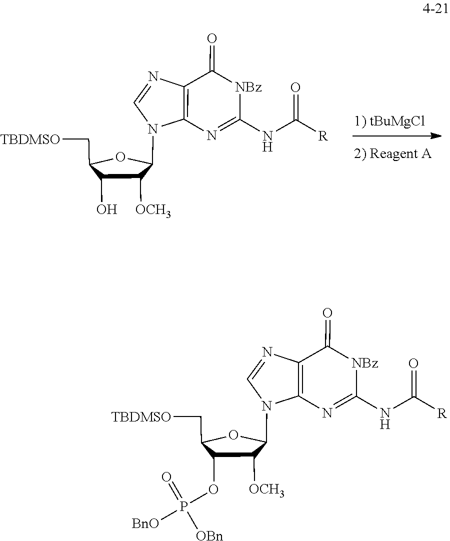

Same procedure as compound 4-20

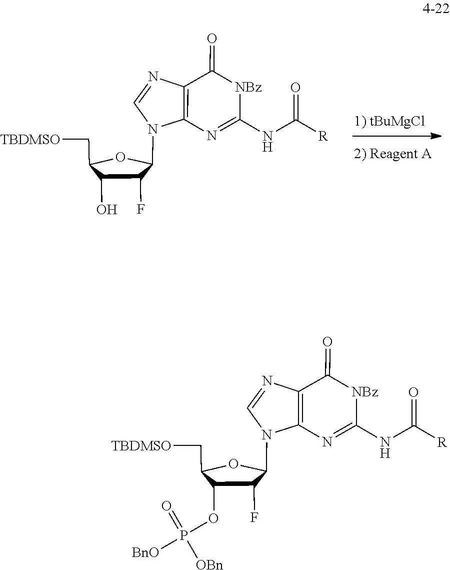

Same procedure as compound 4-20

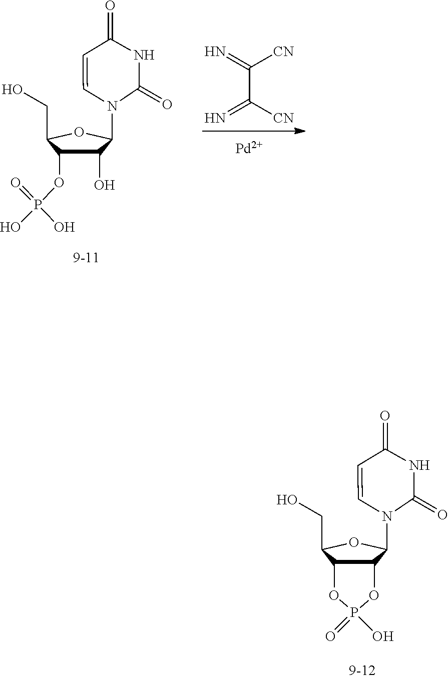

### 1-((3aR,4R,6R,6aR)-2-hydroxy-6-(hydroxymethyl)-2-oxidotetrahydrofuro[3,4-d][1,3,2]dioxaphosphol-4-yl)pyrimidine-2,4(1H,3H)-dione

To a 1 mL aqueous solution containing 2R,3S,4R,5R)-5-(2,4-dioxo-3,4-dihydropyrimidin-1(2H)-yl)-4-hydroxy-2-(hydroxymethyl)tetrahydrofuran-3-yl dihydrogen phosphate (X g, 0.01 M) was added 0.1 M Pd²⁺, 0.2 M imidazole and 0.1 M DISN. The mixture was heated in a screwcap tube for 3 h at 60° C. The mixture was cooled and 74 mg of sodium salt of EDTA was added and reaction mixture centrifuged. Separation was carried out by HPLC using anion exchange column with 0.2 M KH₂PO₄ as solvent to afford 1-((3aR,4R,6R,6aR)-2-hydroxy-6-(hydroxymethyl)-2-oxidotetrahydrofuro[3,4-d][1,3,2]dioxaphosphol-4-yl)pyrimidine-2,4(1H,3H)-dione (42%) (Adv. Space. Res. 3, 9, 61-68).

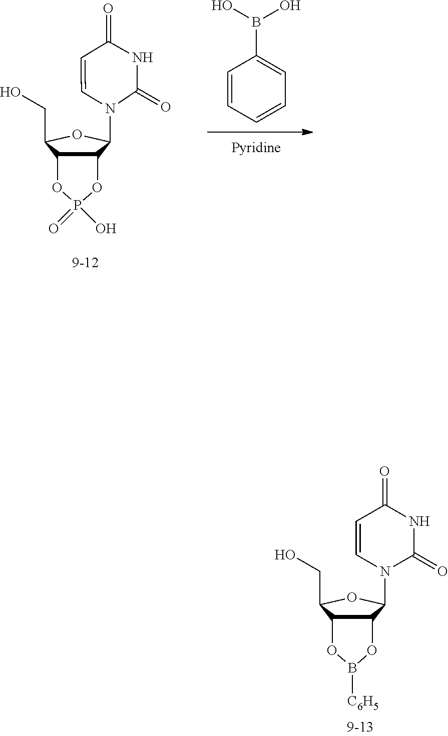

### 1-((3aR,4R,6R,6aS)-6-(hydroxymethyl)-2-phenyltetrahydrofuro[3,4-d][1,3,2]dioxaborol-4-yl)pyrimidine-2,4(1H,3H)-dione

1-((3aR,4R,6R,6aR)-2-hydroxy-6-(hydroxymethyl)-2-oxidotetrahydrofuro[3,4-d][1,3,2]dioxaphosphol-4-yl)pyrimidine-2,4(1H,3H)-dione (X g, 2.0 mM) was stirred in pyridine (100 mL). Meanwhile 2.0 M PBA in 50 mL of pyridine was added to the stirred nucleoside and the reaction was heated under reflux for 2 h. The reaction mixture was concentrated in vacuo, and the residue was washed with ether and re-crystallized to afford 1-((3aR,4R,6R,6aS)-6-(hydroxymethyl)-2-phenyltetrahydrofuro[3,4-d][1,3,2]dioxaborol-4-yl)pyrimidine-2,4(1H,3H)-dione, yield 72% (Tetrahedron 1969, 25, 477-484).

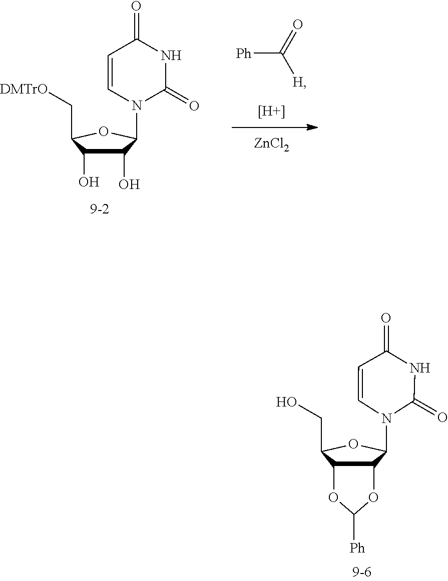

### 1-((3aR,4R,6R,6aR)-6-(hydroxymethyl)-2-phenyltetrahydrofuro[3,4-d][1,3]dioxol-4-yl)pyrimidine-2,4(1 H,3H)-dione

Method K: Benzaldehyde (30 mL) was re-distilled and shielded from the atmosphere. (2R,3R,4S,5R)-2-(6-amino-9H-purin-9-yl)-5-(hydroxymethyl)tetrahydrofuran-3,4-diol (5 g, X mmol) was mixed with 30 mL of benzaldehyde along with 2.5 g of freshly fused zinc chloride and 2 mL of glacial acetic acid. The reaction mixture was stirred at 0° C. for 24 h. The reaction mixture was poured into 200 mL of ice-water and extracted with ether (3×50 mL). Sodium sulfate was used to dry the combined ether extracts and were concentrated to a thin syrup. The compound was added to a mixture of 50 mL of pentane and 60 mL of hexane to obtain 2.5 g of ((3aR,4R,6R,6aR)-6-(6-amino-9H-purin-9-yl)-2-phenyltetrahydrofuro[3,4-d][1,3]dioxol-4-yl)methanol as a crystallize product (29% yield) (JACS 1956, 78. 4715-4717), Chemical Reviews 1979, 79, 6, 491-513.

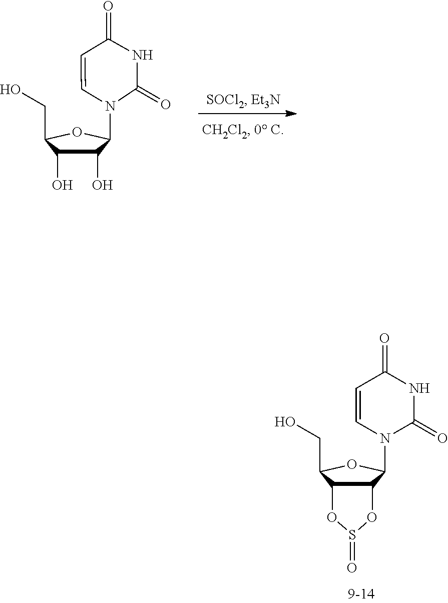

### 1-((3aR,4R,6R,6aR)-6-(hydroxymethyl)-2-oxidotetrahydrofuro[3,4-d][1,3,2]dioxathiol-4-yl)pyrimidine-2,4(1H,3H)-dione

Method N: Uridine (X) was dissolved in 15 mL of methylene chloride under argon, stirred at 0° C. Thionyl chloride (X) in 10 mL of methylene chloride was added dropwise over 40 min, and then the reaction mixture was refluxed for 1 h. The methylene chloride solution was then cooled, washed three times with water, once with saturated bicarbonate solution, and once with water, dried over magnesium sulfate, and distilled under vacuum to yield (85%) of pure cyclic sulfite 9-14 (J. Org. Chem. 1990, 55, 1211-1217)

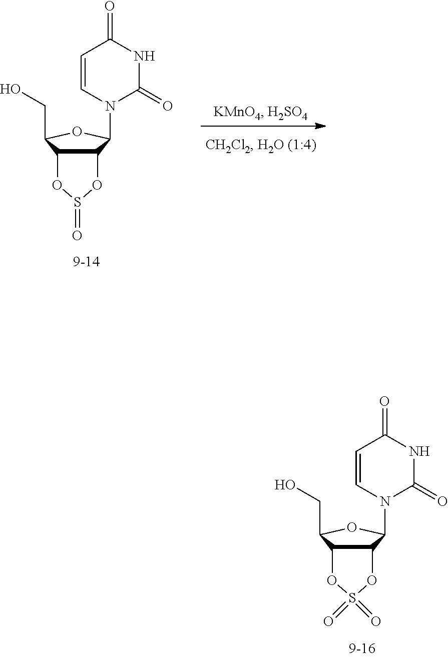

### 1-((3aR,4R,6R,6aR)-6-(hydroxymethyl)-2,2-dioxidotetrahydrofuro[3,4-d][1,3,2]dioxathiol-4-yl)pyrimidine-2,4(1H,3H)-dione

Method O: To the sulfite 9-14 (X mmol) in 50 mL of methylene chloride in an ice bath was added a cold solution of HzS04 (25 g) in 200 mL of water. KMnO, (15 g) was then added in small portions with vigorous stirring. Additional methylene chloride was added as necessary to replace that lost to evaporation. Stirring was continued until no further permanganate color was detectable from a drop of solution on filter paper. Sodium bisulfite was then added in a fume hood slowly and in small portions to entirely dissolve the brown precipitate. The methylene chloride layer was separated, washed with sodium bicarbonate, dried over magnesium sulfate, and evaporated on a rotary evaporator at 35° C. The crude sulfate was then either distilled under reduced pressure or recrystallized from a suitable solvent.

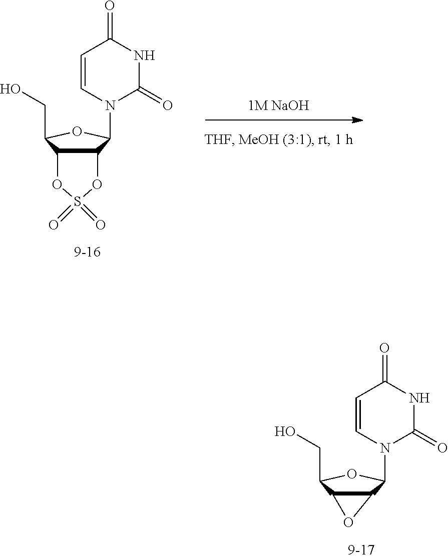

### 1-((1R,2R,4R,5R)-4-(hydroxymethyl)-3,6-dioxabicyclo[3.1.0]hexan-2-yl)pyrimidine-2,4(1H,3H)-dione

Method P: To a solution of a cyclic sulfate (0.4 mmol) in THF (3 mL) and MeOH (1 mL) was added 1 M NaOH (0.8 mL) at room temperature. The reaction mixture was stirred for an appropriate time. The progress of the reaction was monitored by TLC. The reaction mixture was diluted with ether, and washed with 0.1 M HCl, water and brine successively. Drying over anhydrous MgSO, and evaporation of the solvent afforded the crude product 9-17 that was then purified on silica gel.

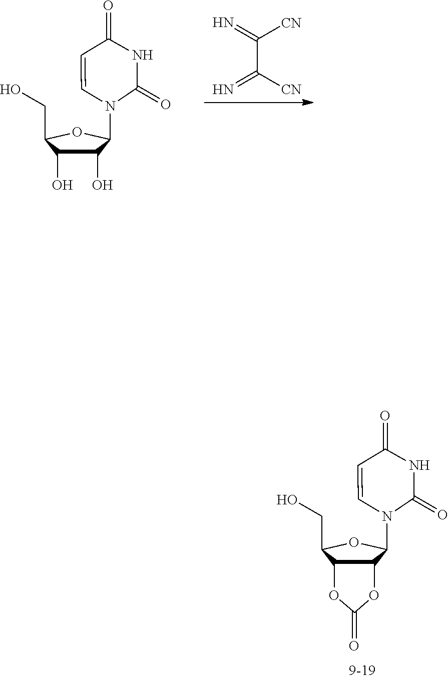

### 1-((3aR,4R,6R,6aR)-6-(hydroxymethyl)-2-oxotetrahydrofuro[3,4-d][1,3]dioxol-4-yl)pyrimidine-2,4(1H,3H)-dione

Method U: A 1 mL aqueous solution containing 0.01M Uridine, 0.1M DISN, and 0.3M imidazole at pH 8 was allowed to react at 2° C. The course of the reaction was monitored by HPLC using a reverse phase column. Once the reaction had reached completion the water was removed under vacuum using a rotary evaporator, placed on silica gel, and purified using flash chromatography.

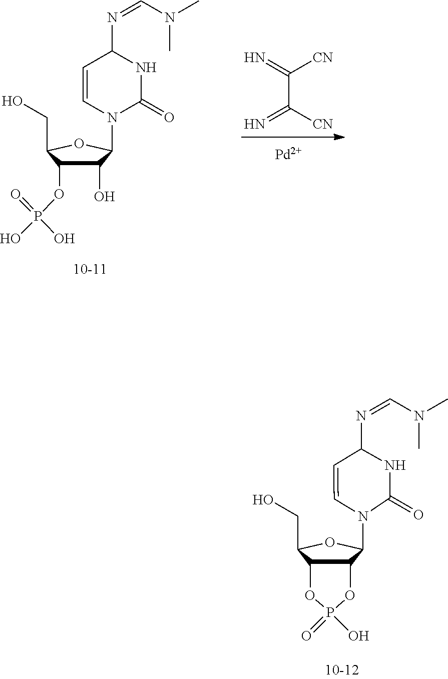

### (Z)-N′-(1-((3aR,4R,6R,6aR)-2-hydroxy-6-(hydroxymethyl)-2-oxidotetrahydrofuro[3,4-d][1,3,2]dioxaphosphol-4-yl)-2-oxo-1,2,3,4-tetrahydropyrimidin-4-yl)-N,N-dimethylformimidamide

### Used Method I.

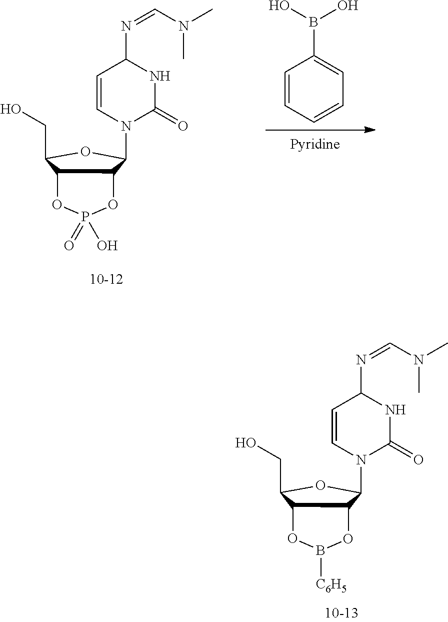

### (Z)-N′-(1-((3aR,4R,6R,6aS)-6-(hydroxymethyl)-2-phenyltetrahydrofuro[3,4-d][1,3,2]dioxaborol-4-yl)-2-oxo-1,2,3,4-tetrahydropyrimidin-4-yl)-N,N-dimethylformimidamide

### Used Method J.

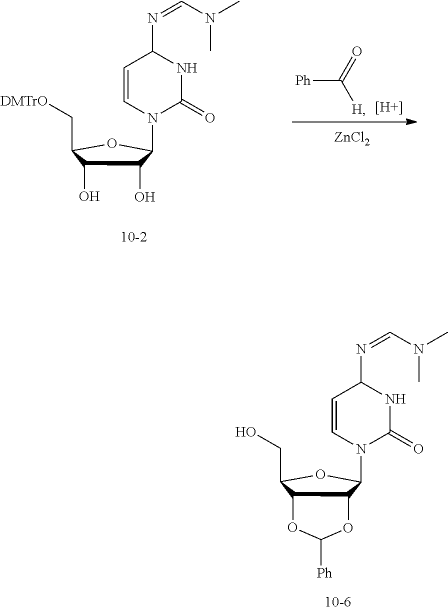

### (Z)-N′-(1-((3aR,4R,6R,6aR)-6-(hydroxymethyl)-2-phenyltetrahydrofuro[3,4-d][1,3]dioxol-4-yl)-2-oxo-1,2,3,4-tetrahydropyrimidin-4-yl)-N,N-dimethylformimidamide

### Used Method K.

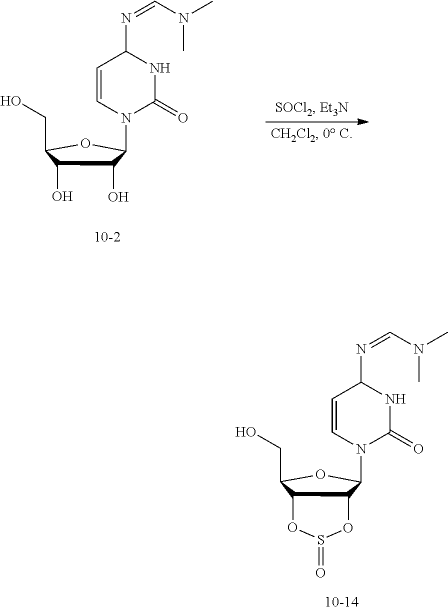

### (Z)-N′-(1-((3aR,4R,6R,6aR)-6-(hydroxymethyl)-2-oxidotetrahydrofuro[3,4-d][1,3,2]dioxathiol-4-yl)-2-oxo-1,2,3,4-tetrahydropyrimidin-4-yl)-N,N-dimethylformimidamide

### Used Method O.

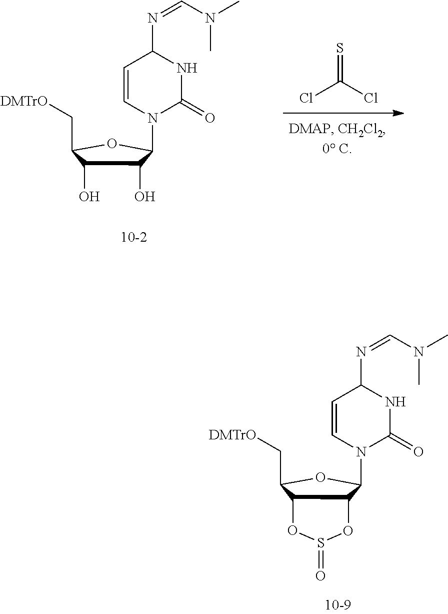

### (Z)-N′-(1-((3aR,4R,6R,6aR)-6-((bis(4-methoxyphenyl)(phenyl)methoxy)methyl)-2-oxidotetrahydrofuro[3,4-d][1,3,2]dioxathiol-4-yl)-2-oxo-1,2,3,4-tetrahydropyrimidin-4-yl)-N,N-dimethylformimidamide

### Used Method P.

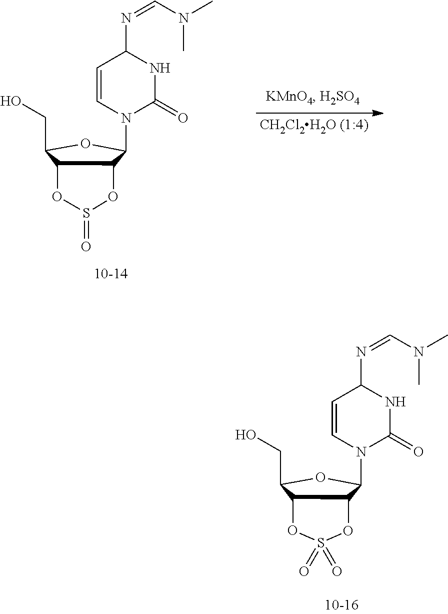

### (Z)-N′-(1-((3aR,4R,6R,6aR)-6-(hydroxymethyl)-2,2-dioxidotetrahydrofuro[3,4-d][1,3,2]dioxathiol-4-yl)-2-oxo-1,2,3,4-tetrahydropyrimidin-4-yl)-N,N-dimethylformimidamide

### Used Method Q.

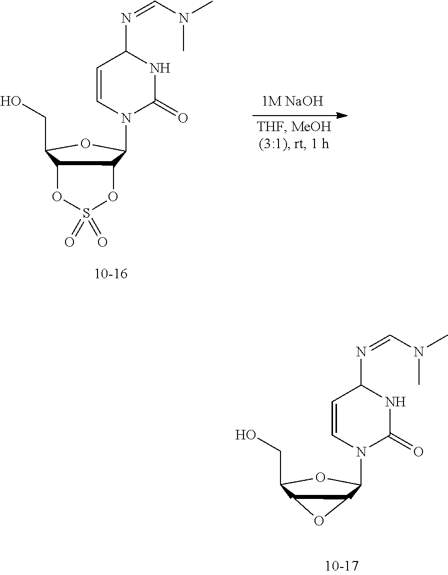

### (Z)-N′-(1-((1R,2R,4R,5R)-4-(hydroxymethyl)-3,6-dioxabicyclo[3.1.0]hexan-2-yl)-2-oxo-1,2,3,4-tetrahydropyrimidin-4-yl)-N,N-dimethylformimidamide

### Used Method R.

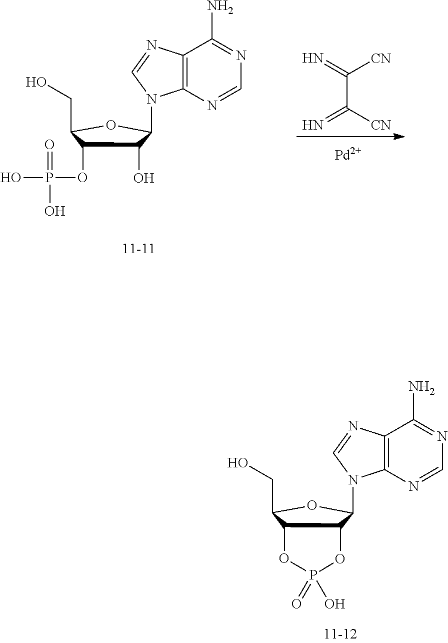

### (3aR,4R,6R,6aR)-4-(6-amino-9H-purin-9-yl)-2-hydroxy-6-(hydroxymethyl)tetrahydrofuro[3,4-d][1,3,2]dioxaphosphole 2-oxide

### Used Method I.

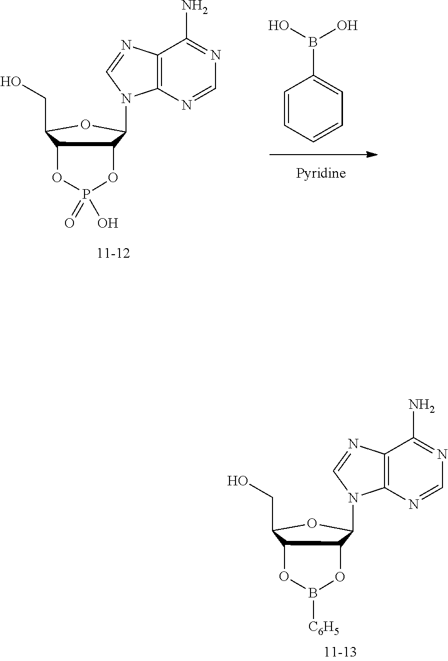

### ((3aS,4R,6S,6aR)-6-(4-amino-7H-pyrrolo[3,2-d]pyrimidin-7-yl)-2-phenyltetrahydrofuro[3,4-d][1,3,2]dioxaborol-4-yl)methanol

### Used Method J.

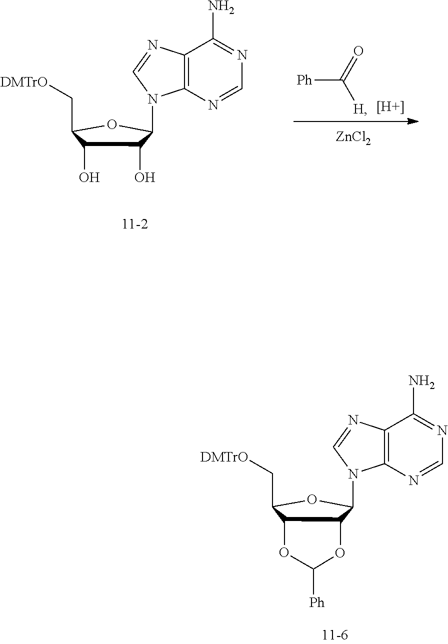

### ((3aR,4R,6R,6aR)-6-(6-amino-9H-purin-9-yl)-2-phenyltetrahydrofuro[3,4-d][1,3]dioxol-4-yl)methanol

### Used Method K.

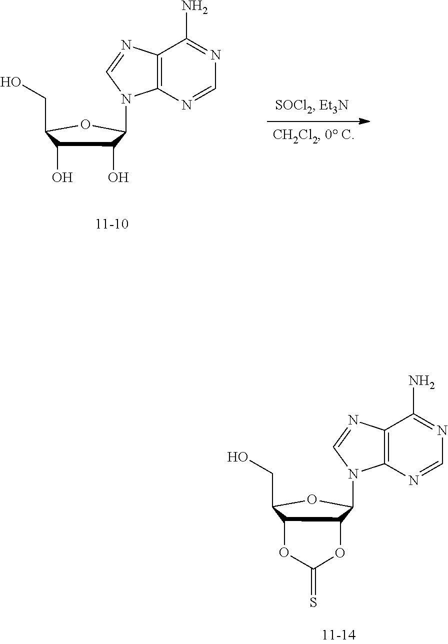

### (3aR,4R,6R,6aR)-4-(6-amino-9H-purin-9-yl)-6-(hydroxymethyl)tetrahydrofuro[3,4-d][1,3]dioxole-2-thione

### Used Method N.

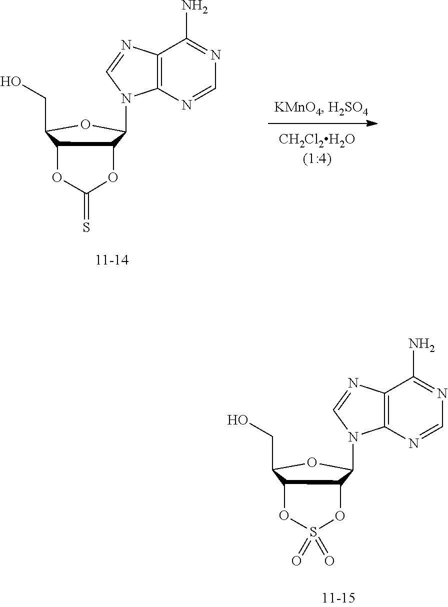

### (3aR,4R,6R,6aR)-4-(6-amino-9H-purin-9-yl)-6-(hydroxymethyl)tetrahydrofuro[3,4-d][1,3,2]dioxathiole 2,2-dioxide

### Used Method P.

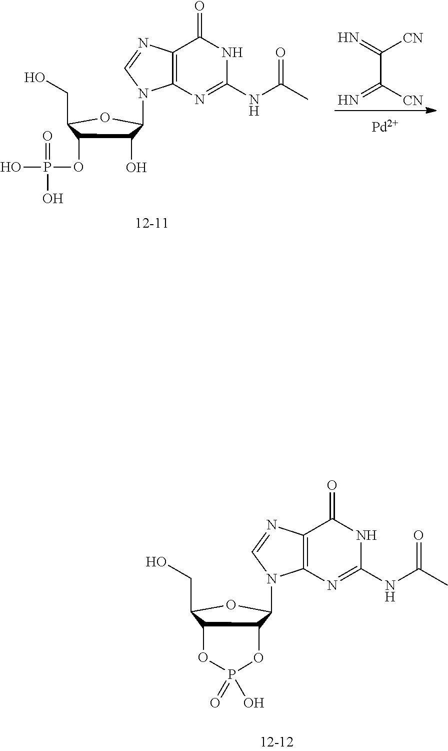

### N-(9-((3aR,4R,6R,6aR)-2-hydroxy-6-(hydroxymethyl)-2-oxidotetrahydrofuro[3,4-d][1,3,2]dioxaphosphol-4-yl)-6-oxo-6,9-dihydro-1H-purin-2-yl)acetamide

### Used Method I.

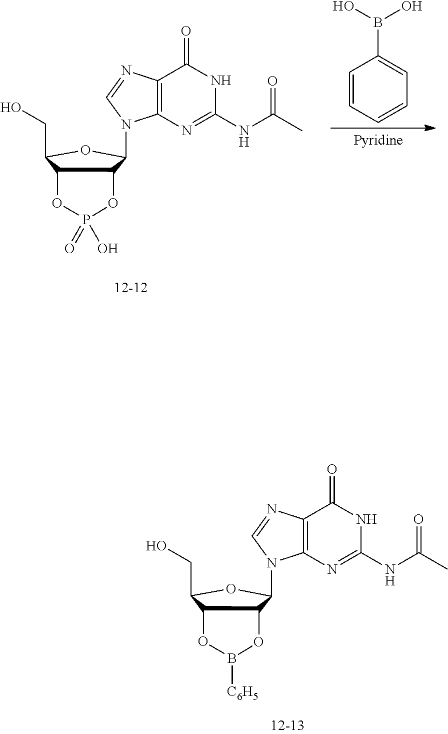

### N-(9-((3aR,4R,6R,6aS)-6-(hydroxymethyl)-2-phenyltetrahydrofuro[3,4-d][1,3,2]dioxaborol-4-yl)-6-oxo-6,9-dihydro-1H-purin-2-yl)acetamide

### Used Method J.

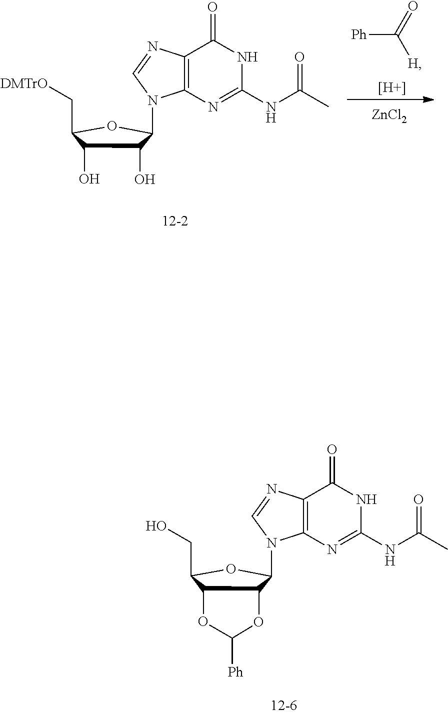

### N-(9-((3aR,4R,6R,6aR)-6-(hydroxymethyl)-2-phenyltetrahydrofuro[3,4-d][1,3]dioxol-4-yl)-6-oxo-6,9-dihydro-1H-purin-2-yl)acetamide

### Used Method K.

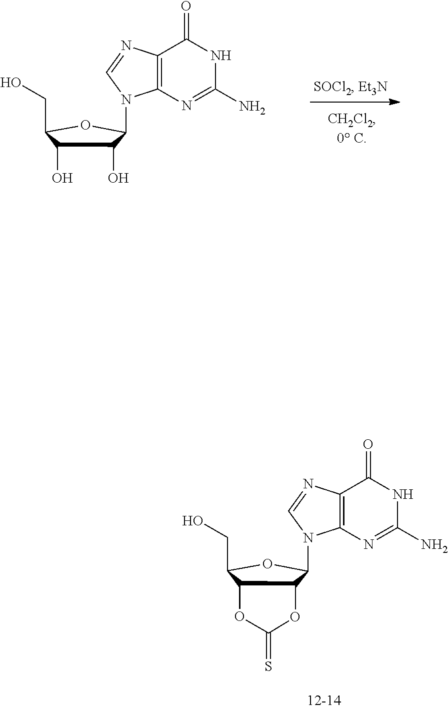

### 2-amino-9-((3aR,4R,6R,6aR)-6-(hydroxymethyl)-2-thioxotetrahydrofuro[3,4-d][1,3]dioxol-4-yl)-1,9-dihydro-6H-purin-6-one

### Used Method N.

### Thiophosphorylation Procedures

### Phosphoryl Chloride Procedure

The triphosphorylation substrate was dissolved in a mixture of dry trimethyl phosphate (1 mL/0.1 mmol of substrate) and dry pyridine (0.6 mL/0.1 mmol substrate) under Ar. The mixture was cooled in an ice bath. A first aliquot of 1.5 equiv of phosphoryl chloride was added in one portion as a neat liquid. Five minutes later, a second aliquot of 1.5 equiv of phosphoryl chloride was added. The mixture was stirred an additional 30 min. A solution of tetrabutylammonium hydrogen diphosphate (4 Eq) in dry DMF was cooled in an ice bath in a vial under Ar. This was added to the rxn mixture dropwise over 60 sec at rxn t=35 min. Immediately N1,N1,N8,N8-tetramethylnaphthalene-1,8-diamine (4 Eq) was added as a solid in one portion. The mixture was stirred for 30 min after this addition and was quenched with 8 mL of cold 0.1 M triethylammonium bicarbonate buffer. The mixture was stirred for 10 min and transferred to a separatory funnel and the solution was extracted 1× with 20 mL of EtOAc. The aq solution was subjected to FPLC using Capto DEAA weak cation exchange column with water as buffer A and 1 M triethyl ammonium bicarbonate (TEAB) as buffer B. The gradient started with 1% B for 15 mins and was increased to 10% B and held for 13 mins and then increased to 40% B and held for 15 mins and it was finally increased to 70% B and held 12 mins. The total run was 81 mins with a flow rate of 20 mL per min.

### Phosphorinone Procedure

The triphosphorylation substrate (0.1 mmol) was dissolved in 1.5 mL of pyridine and 1.0 mL of dioxane and cooled in an ice bath under Ar. 2-chloro-4H-benzo[d][1,3,2]dioxaphosphinin-4-one (1.1 eq) was added as a solid at ice bath T in one portion. The mixture was allowed to stir for 20 min at which time the ice bath was removed and the mixture was allowed to warm to ambient T. After a total reaction time of 1 h, a solution of tetrabutylammonium hydrogen diphosphate (1.1 eq) and tributylamine (1.0 eq) in dry DMF was added via syringe dropwise over about a minute. After 35 min, a standard 0.1 M solution of iodine in pyridine/water/THF was added (1.33 equiv) was added. The mixture was stirred for 20 min after the completion of the addition. A 10% aq solution of sodium thiosulfate was added dropwise until the color had substantially dissipated and the addition of further drops of Na₂SO₃ solution did not cause further dissipation

(ca 1.0 mL). The mixture was stirred for 10 minutes and the solvent was evaporated. The residue on evaporation was partitioned between 5 mL of water and 10 mL of EtOAc. The aq layer was separated and subjected to FPLC separation using Capto DEAA weak cation exchange column with water as buffer A and 1 M triethyl ammonium bicarbonate (TEAB) as buffer B. The gradient started with 1% B for 15 mins and was increased to 10% B and held for 13 mins and then increased to 40% B and held for 15 mins and it was finally increased to 70% B and held 12 mins. The total run was 81 mins with a flow rate of 20 mL per min.

### Hydrogenation Procedure

To a solution of triphosphorylated substrate in a mixture of water and methanol (2:1, v/v) at rt under ambient pressure was added 10% Pd/C and the mixture was hydrogenated for 30 min. The reaction mixture was run through a 0.2 uM PTFE filter and the reaction vial was washed with water three times. The combined washes were subjected to HPLC using a DNA Pac ion pairing column with 6.25 mM triethyl ammonium acetate (TEAA) as buffer A and 1 M TEAA as buffer B. The gradient started with 1% B for 2 mins, and then increased to 20% B and held for 3 mins and then increased to 45% B and held for 3 mins, and then increased to 75% B and held for 3 mins and then increased to 95% B and held for 3 mins and finally brought back to 1% B. The flow rate was 8.5 mL per minute for 30 mins.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for synthesizing an oligoribonucleotide, the method comprising:
exposing a nucleic acid attached to a solid support to a nucleotide analog in the presence of a polymerase and in the absence of a nucleic acid template thereby to incorporate the nucleotide analog into the nucleic acid, wherein, after incorporation, the nucleotide analog comprises a modification that prevents the polymerase from catalyzing addition of a subsequent natural nucleotide or nucleotide analog, and wherein the modification is selected from the group consisting of a 3-O-blocking group and 2′hydroxyl, a 3′-O-blocking group and 2′-modifying group, a 3′-O-blocking group and 2′-O-blocking group, and a 3′-2′ bridged hydroxyl group; and
altering the modification to the incorporated nucleotide analog to permit incorporation of a subsequent natural nucleotide or nucleotide analog under altering conditions that prevent strand scission.

2. The method of claim 1, wherein the altering conditions comprise exposure to an enzyme to remove the 3′-O-blocking group.

3. The method of claim 1, wherein the altering conditions comprise exposure to a non-enzymatic reagent to remove the 3′-O-blocking group.

4. The method of claim 1, wherein the polymerase is polymerase theta or a variant thereof.

5. The method of claim 1, wherein the 3′-O-blocking group comprises one or more selected from the group consisting of ethers, pseudohalogens, nitrates, nitrites, sulfinates, sulfonates, phosphates, phosphites, phosphides, and esters.

6. The method of claim 1, wherein the 2′-O-blocking group comprises one or more selected from the group consisting of ethers, cyanoethyl, nitrates, nitrites, sulfinates, sulfonates, phosphates, phosphites, phosphides, and esters.

7. The method of claim 1, wherein the 2′-modifying group is selected from the group consisting of O-alkyl, methoxy, amino, fluoro, chloro, and bromo.

8. The method of claim 1, wherein the nucleotide analog comprises a base modification selected from the group of Table 1.

9. A method for synthesizing a ribooligonucleotide, the method comprising:
exposing a nucleic acid attached to a solid support to a nucleotide analog in the presence of a polymerase and in the absence of a nucleic acid template to incorporate the nucleotide analog into the nucleic acid, wherein, after incorporation, the nucleotide analog comprises a modification that prevents the polymerase from catalyzing addition of a subsequent natural nucleotide or nucleotide analog, and wherein the modification comprises a 3′-O-blocking group and a 2′-O-modifying group, or a 3′-O-blocking group and a 2′-blocking group, or a 3′-2′ bridged hydroxyl group;
altering the modification to the incorporated nucleotide analog to permit incorporation of a subsequent natural nucleotide or nucleotide analog such that a 2′-O-blocking group remains on the incorporated nucleotide analog;
repeating the exposing and altering steps until a desired ribooligonucleotide is synthesized; and
treating the synthesized ribooligonucleotide to remove all remaining 2′-O-blocking groups from incorporated nucleotide analogs.

10. The method of claim 9, wherein altering the modification comprises exposing the incorporated nucleotide analog to an enzyme to remove the 3′-O-blocking group.

11. The method of claim 9, wherein altering the modification comprises exposing the incorporated nucleotide analog to a non-enzymatic reagent to remove the 3′-O-blocking group.

12. The method of claim 9, where polymerase is polymerase theta or a variant thereof.

13. The method of claim 9, wherein the 3′-O-blocking group is selected from the group consisting of ethers, cyanoethyl, nitrates, nitrites, sulfinates, sulfonates, phosphates, phosphites, phosphides, and esters.

14. The method of claim 9, wherein the 2′-O-blocking group is selected from the group consisting of ethers, cyanoethyl, nitrates, nitrites, sulfinates, sulfonates, phosphates, phosphites, phosphides, and esters.

15. The method of claim 9, wherein the 2′-modifying group is selected from the group consisting of O-alkyl, methoxy, amino, fluoro, chloro, and bromo.

16. The method of claim 9, wherein the nucleotide analog comprises a base modification selected from the group consisting of N1-methyladenosine, N6-methyladenosine, N6-methyl-2-aminoadenosine, 5-methyluridine, N1-methylpseudouridine, pseudouridine, 5-hydroxymethyluridine, 5-methylcytidine, 5-hydroxymethylcytidine, N7-methylguanosine, inosine, 2-thiouridine, 6-thioguanosine, 4-thiourdine, xanthosine, and N,N-dimethyladenosine.
